(19) United States
(12) Patent Application Publication
(10) Pub. No.: US 2004/0259864 A1
(43) Pub. Date: Dec. 23, 2004

(54) Substituted pyrimidinone derivatives as ligands of integrin receptors

(76) Inventors: Herve Geneste, Neuhofen (DE); Andreas Kling, Mannheim (DE); Johann-Christian Zechel, Nussloch (DE); Arnulf Lauterbach, Ludwigshafen (DE); Werner Seitz, Plankstadt (DE); Claudia Isabella Graef, Mannheim (DE); Thomas Subkowski, Ladenburg (DE); Wilfried Hornberger, Neustadt (DE); Rainer Spriestersbach, Ellerstadt (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/469,176

(22) PCT Filed: Feb. 21, 2002
(86) PCT No.: PCT/EP02/01865

(30) Foreign Application Priority Data
Feb. 23, 2001 (DE) 101 08 995.3

(51) Int. Cl. C07D 417/02; C07D 413/02; C 07D 4 3/02

(52) U.S. Cl. 514/227.5; 514/232.2; 514/252.13; 514/326; 514/365; 514/374; 514/397; 544/60; 544/124; 544/359; 546/207

(57) ABSTRACT

The invention relates to novel substituted pyrimidone derivatives which bind to integrin receptors, to their preparation and to their use.

The invention relates to novel compounds which bind to integrin receptors, and to their reparation and use.

Integrins are cell surface glycoprotein receptors which mediate interactions between cells of the same and different types, and between cells and extracellular matrix proteins. They are involved in physiological processes such as, for example, embryogenesis, hemostasis, wound healing, immune response and formation/maintenance of tissue architecture.

Disturbances of the expression of cell adhesion molecule genes, and disturbances of receptor function may contribute to the pathogenesis of a large number of disorders such as, for example, tumors, thromboembolic events, cardiovascular disorders, pulmonary diseases, disorders of the CNS, of the kidney, of the gastrointestinal tract or inflammations.

Integrins are heterodimers each consisting of an α and β transmembrane subunit, which are connected non-covalently. To date, 16 different a and 8 different β subunits and 22 different combinations have been identified.

Integrin αᵥβ₃, also called vitronectin receptor, mediates adhesion to a large number of ligands—plasma proteins, extracellular matrix proteins, cell surface proteins—most of which contain the amino acid sequence RGD (Cell, 1986, 44, 517518; Science 1987, 238, 491497), such as, for example, vitronectin, fibrinogen, fibronectin, von Willebrand factor, thrombospondin, osteopontin, laminin, collagen, thrombin, tenascin, MMP-2, bone sialoprotein II, various viral, fungal, parasitic and bacterial proteins, natural integrin antagonists such as disintegrins, neurotoxins—mambin—and leech proteins—decorsin, omatin—and some non-RGD ligands such as, for example, Cyr-61 and PECAM-1 (L. Piali, J. Cell Biol. 1995, 130, 451460; Buckley, J. Cell Science 1996, 109, 437-445, J. Biol. Chem. 1998, 273, 3090-3096).

Several integrin receptors show cross-reactivity with ligands containing the RGD motif. Thus, integrin α_{IIb}β₃, also called platelet fibrinogen receptor, recognizes fibronectin, vitronectin, thrombospondin, von Willebrand factor and fibrinogen.

Integrin αᵥβ₃ is expressed inter alia on endothelial cells, blood platelets, monocytes/macrophages, smooth muscle cells, some B cells, fibroblasts, osteoclasts and various tumor cells such as, for example, melanomas, glioblastomas, carcinomas of the lung, breast, prostate and bladder, osteosarcomas or neuroblastomas.

Increased expression is observed under various pathological conditions, such as, for example, the prothrombotic state, in cases of vessel injury, tumor growth or metastasis or reperfusion, and on activated cells, in particular on endothelial cells, smooth muscle cells or macrophages.

Involvement of integrin αᵥβ₃ has been detected inter alia in the following pathological states:

Cardiovascular disorders such as atherosclerosis, restenosis after vessel injury, and angioplasty (neointima formation, smooth muscle cell migration and proliferation) (J. Vasc. Surg. 1994, 19, 125-134; Circulation 1994, 90, 2203-2206),

acute kidney failure (Kidney Int. 1994, 46, 10501058; Proc. Natl. Acad. Sci. 1993, 90, 5700-5704; Kidney Int. 1995, 48, 1375-1385),

angiogenesis-associated microangiopathies such as, for example, diabetic retinopathy or rheumatoid arthritis (Ann. Rev. Physiol 1987, 49, 453464; Int. Ophthalmol. 1987, 11, 41-50; Cell 1994, 79, 1157-1164; J. Biol. Chem. 1992, 267, 10931-10934),

arterial thrombosis,

stroke (phase II studies with ReoPro, Centocor Inc., 8th annual European Stroke Meeting),

cancers such as, for example, in tumor metastasis or tumor growth (tumor-induced angiogenesis) (Cell 1991, 64, 327-336; Nature 1989, 339, 58-61; Science 1995, 270, 1500-1502),

osteoporosis (bone resorption after proliferation, chemotaxis and adhesion of osteoclasts to bone matrix) (FASEB J. 1993, 7, 1475-1482; Exp. Cell Res. 1991, 195, 368-375, Cell 1991, 64, 327-336),

high blood pressure (Am. J. Physiol. 1998, 275, H1449-H1454),

psoriasis (Am. J. Pathol. 1995, 147, 1661-1667),

hyperparathyroidism,

Pagers disease (J. Clin. Endocrinol. Metab. 1996, 81, 1810-1820),

malignant hypercalcemia (Cancer Res. 1998, 58, 1930-1935),

metastatic osteolytic lesions (Am. J. Pathol. 1997, 150, 1383-1393),

pathogen protein (for example HIV-1 tat)-induced processes (for example angiogenesis, Kaposi's sarcoma) (Blood 1999, 94, 663-672),

inflammation (J. Allergy Clin. Immunol. 1998, 102, 376-381),

heart failure, CHF, and for

antiviral, antiparasitic, antifungal or antibacterial therapy and prophylaxis (adhesion and internalization) (J. Infect. Dis. 1999, 180, 156-166; J. Virology 1995, 69, 2664-2666; Cell 1993, 73, 309-319).

Because of its key role, pharmaceutical preparations which contain low molecular weight integrin αᵥβ₃ ligands are of great therapeutic and diagnostic value and are used inter alia for the indications mentioned.

Advantageous αᵥβ₃ integrin receptor ligands bind to the integrin 3 receptor with increased affinity.

Particularly advantageous αᵥβ₃ integrin receptor ligands additionally have increased selectivity for the integrin αᵥβ₃ and have less effect on the integrin α_{IIb}β₃ by a factor of at least 10, preferably by a factor of at least 100.

An integrin αᵥβ₃ antagonistic effect has been shown, and a beneficial in vivo effect has been demonstrated, for a large number of compounds such as anti-αᵥβ₃ monoclonal antibodies, peptides containing the RGD binding sequence, natural RGD-containing proteins (for example disintegrins) and low molecular weight compounds (FEBS Letts 1991, 291, 50-54; J. Biol. Chem. 1990, 265, 12267-12271; J. Biol. Chem. 1994, 269, 20233-20238; J. Cell Biol 1993, 51, 206-218; J. Biol. Chem. 1987, 262, 17703-17711; Bioorg. Med. Chem. 1998, 6, 1185-1208).

WO 99/30713 describes 1,3-substituted tetrahydropyrimidin-2(1H)one derivatives and piperidin-2-one derivatives, WO 99/31099 describes 1,3-substituted imidazolin-2-one derivatives, WO 98/35949 describes 2,6-substituted 2H-1,4-benzoxazin-3(4H)one derivatives, WO 9800395 and WO 9723451 describe O-substituted tyrosine derivatives, EP 710657 and EP 741133 describe 3,5-substituted 1,3-oxazolidin-2-ones and WO 97/37655 describes isoindoles as antagonists of the α_{V}β₃ integrin receptor.

WO 00/61551 describes substituted pyrimidinone derivatives as integrin ligands which in fact have good activities and selectivities. Nevertheless there is still a need for further improvement in the activities and selectivities and in the pharmacokinetic properties of the integrin ligands.

It is an object of the present invention to provide novel integrin receptor ligands with advantageous properties, such as improved activities, selectivities and pharmacokinetic properties.

We have found that this object is achieved by compounds of the formula I

B-G-L

where B, G and L have the following meanings:

L is a structural element of the formula I_{L}

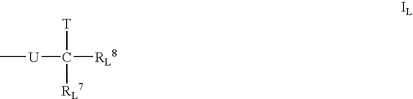

where

T is a COOH group or a radical which can be hydrolyzed to COOH and

—U— is —(CR_{L}¹R_{L}²)ₐ—(V_{L})_{b}—(CR_{L}³R_{L}⁴)_{c}—(W_{L})_{d}—(CR_{L}⁵R_{L}⁶)ₑ—(X_{L})_{f}

where

a, c, e

are, independently of one another, 0, 1, 2 or 3,

b, d, f

are, independently of one another, 0 or 1,

R_{L}¹, R_{L}², R_{L}³, R_{L}⁴, R_{L}⁵, R_{L}⁶ are, independently of one another, hydrogen, halogen, a hydroxyl group, a branched or unbranched, optionally substituted C₁-C₆-alkyl, C₂-C6-alkenyl, C₂-C₆-alkynyl, or C₁-C₆-alkylene-C₃-C₇-cycloalkyl radical, a radical —(CH₂)_{w}—(Y_{L})_{y}—R_{L}⁹, an optionally substituted C₃-C₇-cycloalkyl, aryl, arylalkyl, hetaryl or hetarylalkyl radical or, in each case independently of one another, two radicals R_{L}¹ and R_{L}² or R_{L}³ and R_{L}⁴ or R_{L}⁵ and R_{L}⁶ together are a 3- to 7-membered, optionally substituted, saturated or, unsaturated carbocyclic or heterocyclic system which may contain up to three heteroatoms from the group of O, N or S,

w is 0, 1, 2, 3 or 4,

y is 0 or 1

Y_{L} is —CO—, —CO—N(R_{y}¹)—, —N(R_{Y}¹)—CO—, —N(R_{y}¹)—CO—N(R_{y}¹*)—, —N(R_{y}¹)—CO—O—, —O—, —S—, —SO₂—, —SO₂—N(R_{y}¹)—, —SO₂—O, —CO—O—, —O—CO—, —O—CO—N(R_{y}¹)—, —N(R_{y}¹)— or —N(R_{y}¹)—SO₂—,

R_{y}^{1,} R_{y}¹*

are, independently of one another, hydrogen, a branched or unbranched, optionally substituted C₁-C₆-alkyl, C₁-C₆-alkoxyalkyl, C₂-C₆-alkenyl, C₃-C₁₂-alkynyl, CO-C₁-C₆-alkyl, CO—O-C₁-C₆-alkyl or SO₂-C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂-alkylene-aryl radical,

R_{L}⁷, R_{L}⁸

are, independently of one another, hydrogen, halogen, a hydroxyl group, a branched or unbranched, optionally substituted C₁₋₈-alkyl, C₂-C₆-alkenyl, C₂-C₆-alkynyl or C₁-C₆-alkylene-C₃-C₇-cycloalkyl radical, an optionally substituted —(CH₂)_{w}—R_{L}⁹⁺ radical, an optionally substituted C₃-C₇-cycloalkyl, aryl, arylalkyl, hetaryl or hetarylalkyl radical or the radicals R_{L}⁷ and R_{L}⁸ together are a 3- to 7-membered, optionally substituted, saturated or unsaturated carbocyclic or heterocyclic system which may contain up to three heteroatoms from the group of O, N or S,

R_{L}^{9*} is hydrogen, or a hydroxyl group, CN, halogen, a branched or unbranched, optionally substituted C₁-C₆-alkyl radical, an optionally substituted C₃-C₇-cycloalkyl, aryl, heteroaryl or arylalkyl radical, an optionally C₁-C₄-alkyl or aryl-substituted C₂-C₆-alkynyl or C₂-C₆-alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three identical or different radicals and which may contain up to three different or Identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system to be optionally substituted, or for another, optionally substituted, saturated, unsaturated or aromatic cyclic system to be fused to this cyclic system, or the radical R_{L}⁹ forms together with R_{Y}¹ or R_{Y}^{1*} a saturated or unsaturated C₃-C₇-heterocycle which may optionally contain up to two further heteroatoms selected from the group of O, S or N,

R_{L}^{9*} is hydrogen, or a hydroxyl group, CN, halogen, a branched or unbranched, optionally substituted C₁-C₆alkyl radical, an optionally substituted C₃-C₇-cycloalkyl, aryl, heteroaryl or arylalkyl radical, an optionally C₁-C₄-alkyl or aryl-substituted C₂-C₆-alkynyl or C₂-C₆-alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system to be optionally substituted, or for another, optionally substituted, saturated, unsaturated or aromatic cyclic system to be fused to this cyclic system,

W_{L} is an optionally substituted 4 to 11-membered mono- or polycyclic aliphatic or aromatic hydrocarbon which may contain up to 6 double bonds and up to 6 heteroatoms selected from the group of N, O, S.

V_{L}, X_{L}

are, independently of one another, —CO—, —CO—NR_{L}¹⁰—, —NR_{L}¹⁰—CO—, —S—, —SO—, —SO₂—, —SO₂—NR_{L}¹⁰—, —NR_{L}¹⁰—SO₂—, —CS—, —CS—NR_{L}¹⁰—, —NR_{L}¹⁰—CS—, —CS—O—, O—CS—, —CO—O—, —O—CO—, —O—, ethynylene, —CHR_{L}¹¹—O—CHR_{L}¹²—, —C(═CR_{L}¹¹R_{L}¹²)—, —CR_{L}¹¹═CR_{L}¹²—, CR_{L}¹¹(OR_{L}¹³)—CHR_{L}¹²—, —CHR_{L}¹¹—CR_{L}¹²(OR_{L}¹³)—, —CH(NR_{L}¹⁴—SO₂—R_{L}¹⁵), —CH(NR_{L}¹⁴—CO—R_{L}¹⁵)—, —CH(NR_{L}¹⁴—CO—R_{L}¹⁸)—, CH(NR_{L}¹⁴—CO—NR_{L}^{14′}R_{L}¹⁵)—, —CH(CO—R_{L}¹⁵)—, —CH(CO—OR_{L}¹⁶)— or CH(CO—NR_{L}¹⁴R_{L}¹⁵)—,

R_{L}¹⁰ is hydrogen, a branched or unbranched, optionally substituted C₁-C₆-alkyl, C₁-C₆-alkoxyalkyl, C₁-C₆-alkenyl, C₃-C₁₂-alkynyl, CO—C₁-C₆-alkyl, CO—O-C₁-C₆-alkyl or SO₂C₁-C₆-alkyl radical or an optionally substituted C₃-C₇-cycloalkyl, CO—O-alkylene-aryl, CO-alkylene-aryl, arylalkyl, CO-aryl, SO₂-aryl, hetaryl, CO-hetaryl, hetarylalkyl or SO₂-alkylene-aryl radical, or R_{L}¹⁰ and a radical selected from the group of R_{L}¹, R_{L}², R^{L}³, R_{L}⁴, R_{L}⁵, or R_{L}⁶ together are an optionally substituted 4- to 8-membered heterocycle which may contain up to five identical or different heteroatoms O, N or S,

R_{L}^{11,} R_{L}¹²

are, independently of one another, hydrogen, a hydroxyl group, a branched or unbranched, optionally substituted C₁-C₆-alkyl, C₁-C₄-alkoxy, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,

R_{L}¹³ is hydrogen, a branched or unbranched, optionally substituted C₁-C₆-alkyl, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,

R_{L}¹⁴, R_{L}^{14,}

are, independently of one another, hydrogen, a branched or unbranched, optionally substituted C₁-C₆-alkyl, C₁-C₆-alkoxyalkyl, C₂-C₆-alkenyl, C₃-C₁₂-alkynyl, CO—C₁-C₆-alkyl, CO—O-C₁-C₆-alkyl, C₁-C₆-alkylene-C₃-C₇-cycloalkyl, or SO₂-C₁-C₆-alkyl radical or an optionally substituted C₃-C₇-cycloalkyl, CO—O-alkylene-aryl, CO-alkylene-aryl, aryl, CO-aryl, SO₂-aryl, hetaryl, CO-hetaryl, hetarylalkyl, arylalkyl or SO₂-alkylene-aryl radical,

R_{L}¹⁵ is a branched or unbranched, optionally substituted C₁-C₆-alkyl, alkoxyalkyl, C₁-C₆-alkylene-C₃-C₇-cycloalkyl radical, C₆-C₁₂-bicycloalkyl, alkylene-C₇-C₂₀-tricycloalkyl radical, a C₃-C₇-cycloalkyl, aryl, arylalkyl, hetarylalkyl or 3- to 8-membered, saturated, unsaturated or aromatic heterocyclic radical which may be substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for this cyclic system to be optionally substituted, or for another, optionally substituted, saturated; unsaturated or aromatic cyclic system to be fused to this cyclic system, or the radical R_{L}¹⁵ forms together with R_{L}¹⁴ or R_{L}¹⁴* a saturated or unsaturated C₃-C₇ heterocycle which may optionally contain up to two further heteroatoms selected from the group of O, S or N, and

R_{L}¹⁶ is a branched or unbranched, optionally substituted C₁-C₆-alkyl, alkoxyalkyl or C₁-C₆-alkylene-C₃-C₇-cycloalkyl radical or an optionally substituted C₃-C₇-cycloalkyl, aryl, arylalkyl, hetaryl or hetarylalkyl radical,

G is a structural element of the formula I_{G}

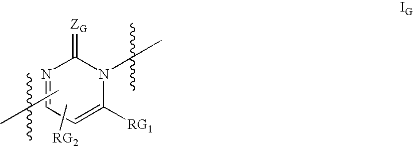

where

the structural element G can be incorporated in both orientations, and

Z_{G} is oxygen, sulfur or NR_{G}³,

R_{G}^{1,} R_{G}²

are, independently of one another, hydrogen, CN, NO₂, halogen, a branched or unbranched, optionally substituted C₁-C₆-alkyl, C₂-C₆-alkenyl or C₂-C₆-alkynyl radical, a branched or unbranched, optionally substituted C₁-C₄-alkylene-OR_{G}⁴, C₁-C₄-alkylene-CO—OR_{G}⁴, C₁-C₄-alkylene-CO—R_{G}⁴, C₁-C₄-alkylene-SO₂—NR_{G}⁵R_{G}⁶, C₁-C₄-alkylene-CO—NR_{G}⁵R_{G}⁶, C₁-C₄-alkylene-NR_{G}⁵R⁶ or C₁-C₄-alkylene-SR_{G}⁴ radical, an optionally substituted-C₃-C₇-cycloalkyl, C₃-C₇-heterocycloalkyl, C₃-C₇-heterocycloalkenyl, alkenyl, C₁-C₄-alkylene-C₃-C₇-cycloalkyl, C₁-C₄-alkylene-C₃-C₇-heterocycloalkyl or C₁-C₄-alkylene-C₃-C₇-heterocycloalkenyl radical, an optionally substituted aryl, arylalkyl, hetaryl or hetarylalkyl radical, an —S—R_{G}⁴, —O—R_{G}⁴, SO—R_{G}⁴, —SO₂—R_{G}⁴, —CO—OR_{G}⁴, —O—CO—NR_{G}⁶R_{G}⁶, —SO₂—NR_{G}⁵R_{G}⁶, —CO—NR_{G}⁵R_{G}⁶, —NR_{G}⁵R_{G}⁶, CO—R_{G}⁴ radical, or R_{G}¹ and R_{G}² together are an optionally substituted, saturated, unsaturated or aromatic 3 to 9-membered carbocyclic, polycarbocyclic, heterocyclic or polyheterocyclic system which may contain up to 4 heteroatoms selected from the group of O, N, S.

R_{G}³ is hydrogen, a hydroxyl group, CN, a branched or unbranched, optionally substituted C₁-C₆-alkyl or C₁-C₄-alkoxy radical or an optionally substituted C₃-C₇-cycloalkyl, —O-C₃-C₇-cycloalkyl radical, aryl, aryl, arylalkyl or —O-alkylene-aryl radical,

R_{G}⁴ is hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cloalkyl, arylalkyl, C₁-C₄-alkylene-heterocycloalkyl, C₁-C₄-alkylene-heterocycloalkenyl or hetarylalkyl radical,

R_{G}⁵, R_{G}⁶

are, independently of one another, hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bisalkylaminoalkylene or acylamino-alkylene radical or an optionally substituted aryl, heterocycloalkyl, heterocycloalkenyl, hetaryl, C₃-C₇-cycloalkyl, C₁-C₄-alkylenen-C₃-C₇-cycloalkyl, arylalkyl, C₁-C₄-alkylene-heterocycloalkyl, C₁-C₄-alkylene-heterocycloalkenyl or hetarylalkyl radical, or an —SO₂—R_{G}⁴, —CO—OR_{G}⁴, —CO—NR_{G}⁴R_{G}⁴* or —CO—R_{G}⁴ radical, and

R_{G}⁴* is an R_{G}⁴ radical independent of R_{G}⁴.

B is a structural element containing at least one atom which can, under physiological conditions, form hydrogen bonds as hydrogen acceptor, where the distance between at least one hydrogen acceptor atom and structural element G along the shortest possible route along the structural element framework is from 4 to 13 atomic linkages,

and the physiologically tolerated salts, prodrugs and enantiomerically pure or diastereomerically pure and tautomeric forms.

A halogen radical means for all radicals and substituents in the present invention for example F, Cl, Br or I, unless mentioned otherwise.

Optionally substituted radicals mean the corresponding unsubstituted and substituted radicals. For all substituted radicals in the present invention if the substituents are not specified in detail then, independently of one another, up to 5 substituents are suitable, for example selected from the following group:

—NO_{2,} —NH₂, —OH, —CN, —COOH, —O—CH₂—COOH, halogen, a branched or unbranched, optionally substituted C₁-C₄-alkyl radical such as, for example, methyl, CF₃, C₂F₅ or CH₂F, a branched or unbranched, optionally substituted —CO—O-C₁-C₄-alkyl, C₃-C₇-cycloalkyl, C₁-C₄-alkoxy, C₁-C₄-thioalkyl, —NH—CO—O-C₁-C₄-alkyl, —O—CH₂—COO-C₁-C₄-alkyl, —NH—CO-C₁-C₄-alkyl, —CO—NH-C₁-C₄-alkyl, —NH—SO₂-C₁-C₄-alkyl, —SO₂—NH-C₁-C₄-alkyl, —N(C₁-C₄-alkyl)₂, —NH—C₁-C₄-alkyl, or —SO₂C₁-C₄-alkyl radical, such as, for example, —SO₂—CF₃, an optionally substituted —NH—CO-aryl, —CO—NH-aryl, —NH—CO—O-aryl, —NH—CO—O-alkylene-aryl, —NH—SO₂-aryl, —SO₂—NH-Aryl, —CO—NH-benzyl, —NH—SO₂-benzyl or —SO₂-NH-benzyl radical, an optionally substituted —SO₂—NR⁴R⁵ or —CO—NR⁴R⁵ radical, it being possible for R⁴ and R⁵ radicals independently of one another to have the meaning as R_{L}¹⁴ hereinafter, or the two R⁴ and R⁵ radicals together being a 3- to 6-membered, optionally substituted, saturated, unsaturated or aromatic heterocycle which, in addition to the ring nitrogen, may contain up to three other different or identical heteroatoms O, N, S and optionally two radicals substituted on this heterocycle together are a fused, saturated, unsaturated or aromatic carbocyclic or heterocyclic system which may contain up to three different or identical heteroatoms O, N, S, and it being possible for the cyclic system optionally to be substituted, or for another, optionally substituted cyclic system to be fused to this cyclic system.

With all terminally bonded, substituted hetaryl and hetarylalkyl radicals in the present invention it is possible, in addition to the aforementioned list of substituents, for two substituents in the hetaryl moiety to form a fused 5- to 7-membered, unsaturated or aromatic carbocyclic system.

T in the structural element L means a COOH group or a radical which can be hydrolyzed to COOH. A radical which can be hydrolyzed to COOH means a radical which is converted into a COOH group after hydrolysis.

A group which may be mentioned as an example of a radical T which can be hydrolyzed to COOH is

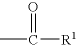

in which R¹ has the following meaning:

a) OM, where M can be a metal cation such as an alkali metal cation such as lithium, sodium, potassium, the equivalent of an alkaline earth metal cation such as calcium, magnesium and barium, or an environmentally compatible organic ammonium ion such as primary, secondary, tertiary or quaternary C₁-C₄-alkylammonium or an ammonium ion, such as, for example, ONa, OK or OLi,

b) a branched or unbranched, optionally halogen-substituted C₁-C₈-alkoxy radical such as, for example, methoxy, ethoxy, propoxy, 1-methylethoxy, butoxy, 1-methylpropoxy, 2-methylpropoxy, 1,1-dimethylethoxy, in particular methoxy, ethoxy, 1-methylethoxy, pentoxy, hexoxy, heptoxy, octoxy, difluoromethoxy, trifluoromethoxy, chlorodifluoromethoxy, 1-fluoroethoxy, 2-fluoroethoxy, 2,2-difluoroethoxy, 1,1,2,2-tetrafluoroethoxy, 2,2,2-trifluoroethoxy, 2-chloro-1,1,2-trifluoroethoxy or pentafluoroethoxy.

c) a branched or unbranched, optionally halogen-substituted C₁-C₄-alkylthio radical such as methylthio, ethylthio, propylthio, 1-methylethylthio, butylthio, 1-methylpropylthio, 2-methylpropylthio or 1,1-dimethylethylthio radical

d) an optionally substituted —O—alkylene-aryl radical such as, for example, —O-benzyl

e) R¹ also a radical —(O)ₘ₁—N(R²)(R³),

in which m1 is 0 or 1, and R² and R³, which may be identical or different, have the following meaning:

hydrogen,

a branched or unbranched, optionally substituted

C₁-C₆-alkyl radical, such as, for example, methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl or the corresponding substituted radicals, preferably methyl, ethyl, propyl, butyl or 1-butyl,

C₂-C₆-alkenyl radical such as, for example, vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl and 1-ethyl-2-methyl-2-propenyl, in particular 2-propenyl, 2-butenyl, 3-methyl-2-butenyl and 3-methyl-2-pentenyl or the corresponding substituted radicals,

C₂-C₈-alkynyl radical such as, for example, ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-3-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably 2-propynyl, 2-butynyl, 1-methyl-2-propynyl or 1-methyl-2-butynyl, or the corresponding substituted radicals,

C₃-C₈-cycloalky, such as, for example, cyclopropyl, cyclobutyl, cyclopentyl, cyclohexyl and cycloheptyl, cyclooctyl or the corresponding substituted radicals,

or a phenyl radical, optionally substituted one or more times, for example one to three times, by halogen, nitro, cyano, C₁-C₄-alkyl, C₁-C₄-haloalkyl, C₁-C₄-alkoxy, C₁-C₄-haloalkoxy or C₁-C₄-alkylthio, such as, for example, 2-fluorophenyl, 3-chlorophenyl, 4-bromophenyl, 2-methylphenyl, 3-nitrophenyl, 4-cyanophenyl, 2-trifluoromethylphenyl, 3-methoxyphenyl, 4-trifluoroethoxyphenyl, 2-methylthiophenyl, 2,4-dichlorophenyl, 2-methoxy-3-methylphenyl, 2,4-dimethoxyphenyl, 2-nitro-5-cyanophenyl, 2,6-difluorophenyl,

or R² and R³ together form a C₄-C₇-alkylene chain which is closed to a ring, is optionally substituted, for example by C₁-C₄-alkyl and may contain a heteroatom selected from the group of oxygen, sulfur or nitrogen, such as, for example, —(CH₂)₄—, —(CH₂)₅—, —(CH₂)₆—, —(CH₂)₇—, —(CH₂)₂—O—(CH₂)₂—, —CH₂—S—(CH₂)₃—, —(CH₂)₂—O—(CH₂)₃—, —NH—(CH₂)₃—, —CH₂—NH—(CH₂)₂—, —CH₂—CH═CH—CH₂—, —CH═CH—(CH₂)₃—, —CO—(CH₂)₂CO— or —CO—(CH₂)₃—CO—.

Preferred T radicals are —COOH, —CO—O—C₁-C₈-alkyl or —CO—O-benzyl.

The coefficients a, c and e in structural element —U— are, independently of one another, 0, 1, 2 or 3, preferably 0, 1 or 2, particularly preferably 0 or 1.

In further, preferred structural elements —U— the total of the coefficients a, c and e is less than 5.

In particularly preferred structural elements —U— the coefficients a, c and e are, independently of one another, 0 or 1.

In further, particularly preferred structural elements —U— the coefficients b, d and f are 0.

The preferred halogen radical for R_{L}¹, R_{L}², R_{L}³, R_{L}⁴, R_{L}⁵, R_{L}⁶, R_{L}⁷ or R_{L}⁸ is F.

Examples of branched or unbranched C₁₋C₆-alkyl radicals for R_{L}¹, R_{L}², R_{L}³, R_{L}⁴, R_{L}⁵, R_{L}⁶, R_{L}⁷ or R_{L}⁸ in structural element L are, independently of one another, methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl, preferably branched or unbranched C₁-C₄-alkyl radicals such as, for example, methyl, ethyl, propyl, 1-methylethyl, butyl, 1-methylpropyl, 2-methylpropyl or 1,1-dimethylethyl, particularly preferably methyl.

A branched or unbranched C₂-C₆-alkenyl radical for

R_{L}¹, R_{L}², R_{L}³, R_{L}⁴, R_{L}⁵, R_{L}⁶, R_{L}⁷ or R_{L}⁸ in structural element L means, independently of one another, for example vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl or 1-ethyl-2-methyl-2-propenyl.

A branched or unbranched C₂-C₆-alkynyl radical for R_{L}¹, R_{L}², R_{L}³, R_{L}⁴, R_{L}⁵, R_{L}⁶, R_{L}⁷ or R_{L}⁸ in structural element L means, independently of one another, for example ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably ethynyl, 2-propynyl, 2-butynyl, 1-methyl-2-propynyl or 1-methyl-2-butynyl, particularly preferably ethynyl.

A C₃-C₇-cycloalkyl radical for R_{L}¹, R_{L}², R_{L}³, R_{L}⁴, R_{L}⁵, R_{L}⁶, R_{L}⁷ or R_{L}⁸ in structural element L means, independently of one another, for example cyclopropyl, cyclobutyl, cyclopentyl, cyclohexyl or cycloheptyl.

Branched or unbranched C₁-C₆-alkylene-C₃-C₇-cycloalkyl radicals are composed, for example, of branched or unbranched C₁-C₆-alkylene radicals and the aforementioned C₃-C₇-cycloalkyl radicals.

Preferred optionally substituted aryl radicals for R_{L}¹, R_{L}², R_{L}³, R_{L}⁴, R_{L}⁵, R_{L}⁶, R_{L}⁷ or R_{L}⁸ in structural element L are, independently of one another, optionally substituted phenyl, 1-naphthyl or 2-naphthyl.

Preferred optionally substituted arylalkyl radicals for R_{L}¹, R_{L}², R_{L}³, R_{L}⁴, R_{L}⁵, R_{L}⁶, R_{L}⁷ or R_{L}⁸ in structural element L are, independently of one another, optionally substituted benzyl or phenethyl.

Hetaryl radicals for R_{L}¹, R_{L}², R_{L}³, R_{L}⁴, R_{L}⁶, R_{L}⁶, R_{L}⁷ or R_{L}⁸ in

structural element L mean, independently of one another, for example radicals such as 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl.

Substituted hetaryl radical for R_{L}¹, R_{L}², R_{L}³, R_{L}⁴, R_{L}⁵, R_{L}⁶, R_{L}⁷ or R_{L}⁸ in structural element L mean, as described above generally for terminal substituted hetaryl radicals, also fused derivatives of the aforementioned hetaryl radicals, such as, for example, indazole, indole, benzothiophene, benzofuran, indoline, benzimidazole, benzothiazole, benzoxazole, quinoline, 2,3-dihydro-1-benzofuran, furo[2,3]pyridine, furo[3,2]pyridine or isoquinoline.

Hetarylalkyl radicals for R_{L}¹, R_{L}², R_{L}³, R_{L}⁴, R_{L}⁵, R_{L}⁶, R_{L}⁷ or R_{L}⁸ in structural element L mean radicals which are composed, for example, of C₁-C₆-alkylene radicals and of the hetaryl radicals described above, such as, for example, the radicals —CH₂-2-pyridyl, —CH₂-3-pyridyl, —CH₂-4-pyridyl, CH₂-2-thienyl, —CH₂-3-thienyl, —CH₂-2-thiazolyl, —CH₂-4-thiazolyl, CH₂-5-thiazolyl, —CH₂—CH₂-2-pyridyl, —CH₂—CH₂-3-pyridyl, —CH₂₋CH₂-4-pyridyl, —CH₂—CH₂-2-thienyl, —CH₂—CH₂-3-thienyl, —CH₂-2-thiazolyl, —CH₂—CH₂-4-thiazolyl, or —CH₂—CH₂-5-thiazolyl.

It is further possible for two radicals R_{L}¹ and R_{L}² or R_{L}³ and R_{L}⁴ or R_{L}⁵ and R_{L}⁶ or R_{L}⁷ and R_{L}⁸ in each case independently of one another together to be a 3- to 7-membered, optionally substituted, saturated or unsaturated carbocyclic or heterocyclic system which may contain up to three heteroatoms from the group of O, N or S.

The —(CH₂)_{w}—(Y_{L})_{y}—R_{L}⁹ radical, which the radicals R_{L}¹, R_{L}², R_{L}³, R_{L}⁴, R_{L}⁵ and R_{L}⁶ can be independently of one another, is composed of a C₀-C₄-alkylene radical, optionally a linking element Y_{L} selected from the group of

—CO—, —CO—N(R_{y}¹)—, —N(R_{y}¹)—CO—, —N(R_{y}¹)—CO—N(R_{Y}¹)—CO—N(R_{y}¹*)—, —N(R_{y}¹)—CO—O—, —O—, —S—, —SO₂—, —SO₂—N(R_{y}¹)—, —SO₂—O—, —CO—O—, —O—CO—N(R_{y}¹)—, —N(R_{y}¹) or —N(R_{y}¹)—SO₂— and the R_{L}⁹ radical, where

R_{y}^{1,} R_{y}¹*

are, independently of one another, hydrogen, a branched or unbranched, optionally substituted C₁-c₆-alkyl, C₁-C₆-alkoxyalkyl, C₂-C₆-alkenyl, C₃-C₁₂-alkynyl, CO—C₁-C₆-alkyl, CO—O-C₁-C₆-alkyl or SO₂-C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO—hetaryl or SO₂-alkylene-aryl radical, preferably hydrogen, methyl, cyclopropyl, allyl or propargyl, particularly preferably hydrogen or methyl, and

R_{L}⁹

is hydrogen, a hydroxyl group, CN, halogen, a branched, or unbranched optionally substituted C₁-C₆-alkyl radical, an optionally substituted C₃-C₇-cycloalkyl, aryl, heteroaryl or arylalkyl radical, an optionally C₁₋C₄alkyl- or aryl-substituted C₂-C₆-alkynyl or C₂-C₆-alkenyl radical, an optionally substituted C₆-C₁₂-bicycloalkyl, C₁-C₆-alkylene-C₆-C₁₂-bicycloalkyl, C₇C₂₀-tricycloalkyl or C₁-C₆-alkylene-C₇-C₂₀-tricycloalkyl radical, or a 3- to 8-membered, saturated or unsaturated heterocycle which is substituted by up to three identical or different radicals and which may contain up to three different or identical heteroatoms O, N, S, It being possible for two radicals together to be a fused, saturated, unsaturated or aromatic carbocyclic or heterocyclic system which may contain up to three different or identical heteroatoms O, N, S, and it being possible for the cyclic system optionally to be substituted, or for another, optionally substituted, saturated, unsaturated or aromatic cyclic system to be fused to this cyclic system, such as, for example, optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, isothiazolyl, 2-imidazolyl, 4-4-imidazolyl, 5-imidazolyl, 3-pyridazinyl, 4-pyridazinyl, 5-pyridazinyl, 6-pyridazinyl, 2-(1,3,4)-thiadiazolyl, 2-(1,3,4)-oxadiazolyl, 3-isoxazolyl, 4-isoxazolyl, 5 isoxazolyl or triazinyl.

It is also possible for R_{L}⁹ and R_{y}¹ or R_{y}¹* together to form a saturated or unsaturated C₃*C₇-heterocycle which may optionally contain up to two other heteroatoms selected from the group of O, S or N.

The radicals R_{L}⁹ and R_{y}¹ or R₆₅ ¹* preferably together form a cyclic amine as C₃-C₇-heterocycle in the case where the radicals are bonded to the same nitrogen atom, such as, for example, N-pyrrolidinyl, N-piperidinyl, N-hexahydroazepinyl, N-morpholinyl or N-piperazinyl, it being possible for the free amine protons on heterocycles having free amine protons, such as, for example, N-piperazinyl, to be replaced by conventional amine protective groups such as, for example, methyl, benzyl, Boc (tert-butoxycarbonyl), Z (benzyloxycarbonyl), tosyl, —SO₂—C₁-C₄-alkyl, —So₂-phenyl or —SO₂-benzyl.

Preferred —(CH₂)_{w}—(Y_{L})_{Y}—R_{L}⁹ radicals for R_{L}¹, R_{L}³ or R_{L}⁵ in structural element L are optionally substituted side chains of natural amino acids, preferably optionally substituted side chains of the amino acids Ser, Thr, Tyr, Asp, Asn, Glu, Gin, Cys, Met, Lys or Om, optionally substituted side chains of unnatural amino acids as described, for example, in catalogs of the companies Bachem 1999, Novabiochem 1999, Neosystem 1997/98 and Advanced Chem Tech 1999.

Side chains of natural α-amino acids mean the side chains including the β carbon atom. Examples of unnatural amino acids are β-amino acids. In this case, side chains mean the side chains including the γ carbon atom. Substituted side chains also mean, for example, side chains which have a protective group on a functional group in the side chain such as, for example, —NH₂, —SH, —OH or —COOH.

The optionally substituted radical —(CH₂)_{w}—R_{L}^{9*}, which the radicals R_{L}⁷ and R_{L}⁸ can be independently of one another, is composed of an optionally substituted C₀-C₄-alkyl radical and the radical R_{L}^{9*}, where R_{L}⁹* has the following meaning:

hydrogen, a hydroxyl group, CN, halogen, a branched or unbranched, optionally substituted C₁-C₆-alkyl radical, an optionally substituted C₃-C₇-cycloalkyl, aryl, preferably phenyl or naphthyl, heteroaryl or arylalkyl radical, an optionally C₁-C₅-alkyl- or aryl-substituted C₂-C₆-alkynyl or C₂-C₆-alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocycle which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system optionally to be substituted, or for another, optionally substituted, saturated, unsaturated or aromatic cyclic system to be fused to this cyclic system, such as, for example, optionally substituted 2-pyridyl, 3-pyridyl 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 2(1,3,4)-thiadiazolyl, 2(1,3,4)-oxadiazolyl, 3-isoxazolyl, 4-isoxazolyl, 5-isoxazolyl or triazinyl.

In particularly preferred radicals for R_{L}¹, R_{L}², R_{L}³, R_{L}⁴, R_{L}⁵, R_{L}⁶, R_{L}⁷ or R_{L}⁸ in structural element L, in each case independently of one another one of the radicals R_{L}¹ and R_{L}² or R_{L}³ and R_{L}⁴ or R_{L}⁵ and R_{L}⁶ or R_{L}⁷ and R_{L}⁸ is hydrogen or methyl.

An optionally substituted 4- to 11-membered mono- or polycyclic aliphatic or aromatic hydrocarbon which may contain up to 6 double bonds and up to 6 heteroatoms selected from the group of N, O, S for W_{L} preferably means optionally substituted arylene such as, for example, optionally substituted phenylene or naphthylene, optionally substituted hetarylene such as, for example, the radicals

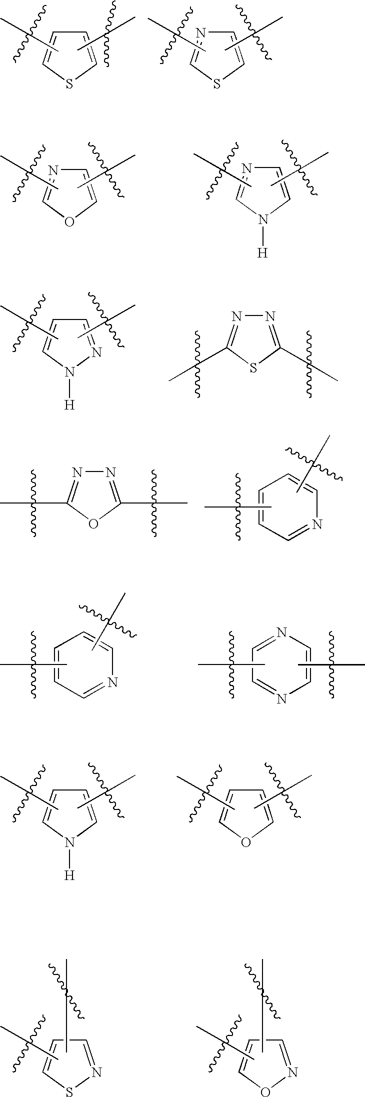

and their substituted or fused derivatives, or radicals of the formula I_{WL} to III_{WL},

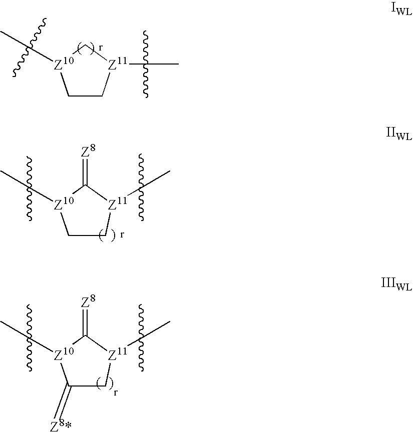

where the radicals can be incorporated in both orientations, the coefficient r is 0, 1, 2 or 3, and Z¹⁰ and Z¹¹ are, independently of one another, CH or nitrogen, and Z⁸ and Z⁸*, independently of one another, are oxygen, sulfur or NH.

W_{L} is preferably an optionally substituted phenylene radical, a radical

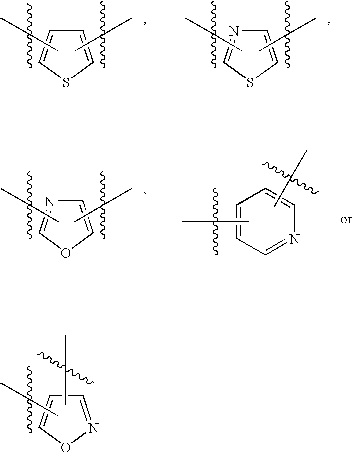

or their substituted or fused derivatives, or radicals of the formula I_{WL} to III_{WL},

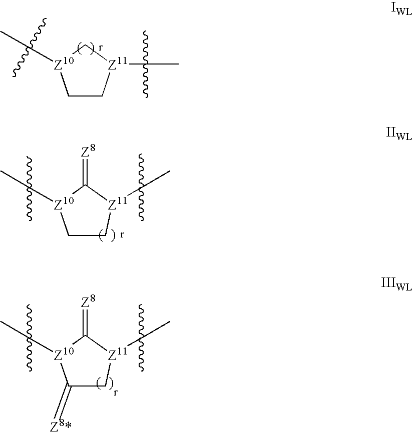

where the radicals can be incorporated in both orientations, the coefficient r is 0, 1, 2 or 3, and Z¹⁰ and Z¹¹ are, independently of one another, CH or nitrogen and Z⁸ and Z⁸*, independently of one another, are oxygen, sulfur or NH.

Z⁸ in preferred radicals of the formula II_{WL} or III_{WL} for W_{L} is oxygen.

Preferred radicals for V_{L} and X_{L} are, independently of one another, —CO—NR_{L}¹⁰—, —NR_{L}¹⁰—CO—, —SO₂NR_{L}¹⁰—, —NR_{L}¹⁰—SO₂—, —O—, —CH(NR_{L}¹⁴—SO₂—R_{L}¹⁵)—, —CH(NR_{L}¹⁴—CO—R_{L}¹⁵)—, —CH(NR_{L}¹⁴—CO—OR_{L}¹⁶)—, CH(NR_{L}¹⁴—CO—NR_{L}^{14′}R_{L}¹⁵)—, —CH(CO—R_{L}¹⁵)—, —CH(CO—R_{L}¹⁶)— and CH(CO—NR_{L}¹⁴R_{L}¹⁵)—.

Particularly preferred radicals for V_{L} and X_{L} are, independently of one another, —CH(NR_{L}¹⁴—SO₂—R_{L}¹⁵)—, —CH(NR_{L}¹⁴—CO—R_{L}¹⁵)—, —CH(NR_{L}¹⁴—CO—OR_{L}¹⁵)—, CH(NR_{L}¹⁴—CO—NR_{L}^{14′}R_{L}¹⁵)—, —CH(CO—R_{L}¹⁵)—, —CH(CO—OR_{L}¹⁵)— and CH(CO—NR_{L}¹⁴—NR_{L}¹⁵)—.

The RL₁₀ radical in structural element L is hydrogen,

branched or unbranched, optionally substituted C₁-C₆-alkyl radical, for example as described above for R_{L}¹, preferably methyl,

C₁-C₆-alkoxyalkyl radical, for example methoxymethyl, ethoxymethyl, t-butoxymethyl, methoxyethyl or ethoxyethyl,

C₂-C₆-alkenyl radical, for example as described above for R_{L}¹ preferably allyl,

C₃-C₁₂-alkynyl radical, for example 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-2-butynyl, 1-ethyl-3-butynyl, 2-ethyl-3-butynyl and 1-ethyl-1-methyl-2-propynyl, preferably propargyl,

or CO—C₁-C₆-alkyl, CO—O—C₁-C₆-alkyl or SO₂-C₁-C₆-alkyl radical which is composed in each case of the corresponding CO—, CO—O— or SO₂— group, and, for example, of the C₁-C₆-alkyl radicals described above,

an optionally substituted

C₃-C₇-cycloalkyl, arylalkyl, hetaryl or hetarylalkyl radical as described, for example, in each case for R_{L}¹ above,

an optionally substituted CO—O-alkylene-aryl-, CO-alkylene-aryl, CO-aryl, SO₂-aryl, CO-hetaryl or SO₂-alkylene-aryl radical which is composed in each case of the corresponding CO—, CO—O— or SO₂— group and, for example, of the corresponding arylalkyl, aryl, hetarylalkyl and hetaryl radicals as described for R_{L}¹.

It is also possible for R_{L}¹⁰ and a radical selected from the group of R_{L}¹, R_{L}², R_{L}³, R_{L}⁴, R_{L}⁵ or R_{L}⁶ together to form an optionally substituted 4- to 8-membered heterocycle which may contain up to five identical or different heteroatoms O, N or S.

Particularly preferred radicals for R_{L}¹⁰ are hydrogen, methyl, cyclopropyl, allyl and propargyl.

A branched or unbranched C₁-C₆-alkyl, C₂-C₆-alkynyl or C₁-C₆-alkylene C₃-C₇-cycloalkyl radical or an optionally substituted C₃-C₇-cycloalkyl, aryl, arylalkyl, hetaryl or hetarylalkyl radical for R_{L}¹¹ or R_{L}¹² means independently of one another, for example the corresponding radicals mentioned above for R_{L}¹.

A branched or unbranched C₁-C₄-alkoxy radical for R_{L}¹¹ or R_{L}¹² means, independently of one another, for example the radicals methoxy, ethoxy, propoxy, 1-methylethoxy, butoxy, 1-methylpropoxy, 2-methylpropoxy or 1,1-dimethylethoxy.

A branched or unbranched C₁-C₆-alkyl, C₂-C₆-alkenyl, C₂-C₆-alkynyl or C₁-C₆-alkylene-C₃-C₇-cycloalkyl radical or an optionally substituted, C₃-C₇-cycloalkyl, aryl, arylalkyl, hetaryl or hetarylalkyl radical for R_{L}¹³ mean, for example, the corresponding radicals mentioned above for R_{L}¹.

Preferred radicals for R_{L}¹⁴ and R_{L}^{14,} are, independently of one another, hydrogen, a branched or unbranched, optionally substituted C₁-C₆-alkyl or C₃-C₁₂-alkynyl radical or an optionally substituted C₃-C₇-cycloalkyl, aryl, hetaryl or arylalkyl radical.

Particularly preferred radicals for R_{L}¹⁴ and R_{L}^{14,} are, independently of one another, hydrogen, methyl, cyclopropyl, allyl or propargyl.

R_{L}¹⁵ is a branched or unbranched, optionally substituted

C₁-C₆-alkyl radical as described above for R_{L}¹, preferably a branched or unbranched, optionally substituted C₁-C₄-alkyl radical, particularly preferably n-butyl, 2-methylpropyl, 1-methylethyl,

alkoxyalkyl radical as described above for R_{L}^{10,}

C₁-C₆-alkylen-C₃-C₇-cycloalkyl radical as described above for R_{L}^{1,} preferably —CH₂-C₃-C₇-cycloalkyl or —CH₂—CH₂-C₃-C₇-cyloalkyl,

C₁-C₁₂-bicycloalkyl or C₇-C₂₀-tricycloalkyl radical such as, for example, bicyclo[4.4.0]decanyl, bicyclo[2.2.2]octanyl, bicyclo-[3.2.1]octanyl, indanyl, adamantyl, norbornyl, noradamantyl or camphor-10-yl,

C₁-C₆-alkylene-C₆-C₁₂-bicycloalkyl or C₁-C₆-alkylene-C₇-C₂₀-tricycloalkyl radical, such as, for example, —CH₂-bicyclo[4.4.0]decanyl, —CH₂-bicyclo[2.2.2]octanyl, —CH₂-bicyclo[3.2.1]octanyl, —CH₂-indanyl, —CH₂-adamantyl, —CH₂-norbornyl, —CH₂-noradamantyl or —CH₂-camphor-10-yl,

a C₃-C₇-cycloalkyl, aryl, arylalkyl or hetarylalkyl radical as described above for R_{L}¹, which is substituted by up to three identical or different radicals,

or a 3- to 8-membered, saturated, unsaturated or aromatic heterocyclic radical which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for this cyclic system optionally to be substituted, or for another, optionally substituted, saturated, unsaturated or aromatic cyclic system to be fused to this cyclic system.

It is furthermore possible for R_{L}¹⁵ and R¹⁴ or R_{L}^{14,} together to form a saturated or unsaturated C₃-C₇-heterocycle which may optionally contain up to two other heteroatoms selected from the group O, S or N.

The radicals R_{L}¹⁵ and R_{L}¹⁴ or R_{L}¹⁴* preferably together form a cyclic amine residue as C₃-C₇-heterocycle in the case where the radicals are bonded to the same nitrogen atom, such as, for example, N-pyrrolidinyl, N-piperidinyl, N-hexahydroazepinyl, N-morpholinyl or N-piperazinyl, it being possible for the free amine protons on heterocycles having free amine protons, such as, for example, N-piperazinyl, to be replaced by conventional amine protective groups such as, for example, methyl, benzyl, Boc (tert-butoxy-carbonyl), Z (benzyloxycarbonyl), tosyl, —SO₁-C₁-C₄-alkyl, —SO₂-phenyl or —SO₂-benzyl. The cyclic amine residue NR_{L}¹⁵R_{L}¹⁴ or NR_{L}¹⁵R_{L}¹⁴* may also, depending on the structural element V_{L} or X_{L}, be a constituent of an amide, sulfonamide, urethane or other possible structural element

Preferred radicals for R_{L}¹⁵ are a branched or unbranched, optionally substituted C₁-C₄-alkyl or —CH₂-C₅-C₇-cycloalkyl radical, an optionally substituted C₅-C₇-cycloalkyl, phenyl, 1-naphthyl, 2-naphthyl, —CH₂-naphthyl, pyridyl, —CH₂-pyridyl, phenethyl, thienyl, —CH₂-thienyl, oxazolyl, —CH₂-oxazolyl, isoxazolyl, —CH₂-isoxazolyl, quinolinyl, isoquinolinyl, —CH₂-quinolinyl, —CH₂-isoquinolinyl, adamantyl, —CH₂-adamantyl, norbornyl, —CH₂-norbornyl, camphor-10-yl or —CH₂-camphor-10-yl radical.

A branched or unbranched, optionally substituted C₁-C₆-alkyl, alkoxyalkyl or C₁-C₆-alkylene-C₃-C₇-cycloalkyl radical or an optionally substituted C₃-C₇-cycloalkyl, aryl, arylalkyl, hetaryl or hetarylalkyl radical for R_{L}¹⁸ means, for example, the corresponding radicals mentioned above for R_{L}¹⁵, preferably hydrogen, a branched or unbranched, optionally substituted C₁-C₄-alkyl or —CH₂-C₅-C₇-cycloalkyl radical, an optionally substituted C₅-C₇-cycloalkyl, phenyl, 1-naphthyl, 2-naphthyl, —CH₂-naphthyl, benzyl, pyridyl, —CH₂-pyridyl, phenethyl, thienyl, —CH₂-thienyl, oxazolyl, —CH₂-oxazolyl, isoxazolyl, —CH₂-isoxazolyl, adamantyl or —CH₂-adamantyl radical.

Particularly preferred radicals for R_{L}¹⁶ are a branched or unbranched, optionally substituted C₁-C₄-alkyl radical and optionally substituted benzyl.

In a further preferred embodiment of the structural element —U—, the radicals R_{L}¹, R_{L}², R_{L}³, R_{L}⁴, R_{L}⁵ or R_{L}⁶ are, independently of one another, hydrogen or methyl and the indices b, d and f are 0 or 1, with the proviso that the radical V_{L} or X_{L} are, independently of one another, a radical —CH(NR_{L}¹⁴—SO₂—R_{L}¹⁵)—, —CH(NR_{L}¹⁴—CO—R_{L}¹⁵)—, —CH(NR_{L}¹⁴—CO—OR_{L}¹⁶)—, —CH(NR_{L}¹⁴—CO—NR_{L}^{14′}R_{L}¹⁵)—, —CH(CO—OR_{L}¹⁶)— or —CH(CO—NR_{L}¹⁴RL¹⁵)—.

A particularly preferred structural element —U— is the methylene group —CH₂—.

Particularly preferred radicals for R_{L}⁷ and R_{L}⁸ are, independently of one another, hydrogen, a branched or unbranched, optionally substituted C₁-C₆-alkyl, C₂-C₆-alkenyl, C₂-C₆-alkynyl or C₁-C₆-alkylene-C₁-C₆-cycloalkyl radical, an optionally substituted —(CH₂)_{w}—R_{L}^{9*} radical, or an optionally substituted aryl, arylalkyl, hetaryl or hetarylalkyl radical.

In a further preferred embodiment, the radical R_{L}⁷ is hydrogen and the radical R_{L}⁸ is an optionally substituted —(CH₂)_{w}—R_{L}⁹* radical, where w is preferably 1, 2 or 3, particularly preferably 1 or 2, and R_{L}^{9*} is an optionally substituted aryl radical, preferably optionally substituted phenyl or naphthyl.

Preferred structural elements L are composed of at least one preferred radical of the radicals belonging to structural element L, while the remaining radicals may vary widely.

Particularly preferred structural elements L are composed of the preferred radicals of the structural element L.

G is a structural element of the formula I_{G}

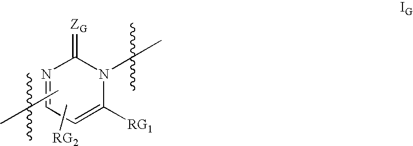

where the structural element G can be incorporated in both orientations.

Z_{G} is oxygen, sulfur or NR_{G}³, preferably oxygen.

In a preferred embodiment of the structural element G, the substitution pattern is fixed as in formula I_{GB}

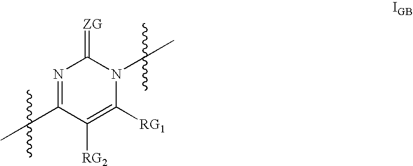

where the structural element G can be incorporated in both orientations in this case too.

In a particularly preferred embodiment of the structural element G, the substitution pattern is fixed as in formula I_{GB}, and the structural element G is incorporated so that the structural element E is connected to the position 4 carbon and the structural element L is connected to the position 1 nitrogen.

R_{G}¹ and R_{G}² in structural element G are, independently of one another, hydrogen, CN, NO₂, halogen, a branched or unbranched, optionally substituted

C₁-C₆-alkyl radical such as, for example, optionally substituted methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl,

C₂-C₆-alkenyl radical such as, for example, optionally substituted vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-3-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butonyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl or 1-ethyl-2-methyl-2-propenyl,

C₂-C₆-alkynyl radical such as, for example, optionally substituted ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl or 1-ethyl-1-methyl-2-propynyl,

a branched or unbranched, optionally substituted C₁-C₄-alkylene-O—R_{G}⁴, C₁-C₄-alkylene-CO—OR_{G}⁴, C₁-C₄-alkylene-CO—R_{G}⁴, C₁C₄-alkylene-SO₂—NR_{G}⁵R_{G}⁶, C₁-C₄-alkylene-CO—NR_{G}⁵R_{G}⁵, C₁-C₄-alkylene-NR_{G}⁵R_{G}⁶ or C₁-C₄-alkylene-SR_{G}⁴ radical, which are composed of branched or unbranched, optionally substituted C₁-C₄-alkylene radicals such as, for example, methylene, ethylene, propylene, n-butylene, isobutylene or t-butylene, the appropriate , —O—, —CO—, —S—, —N groups and the terminal R_{G}⁴, R_{G}⁵ and R_{G}⁶ radicals described below,

an optionally substituted

C₃-C₇-cycloalkyl radical such as, for example, optionally substituted cyclopropyl, cyclobutyl, cyclopentyl, cyclohexyl or cycloheptyl,

C₃-C₇-heterocycloalkyl radical such as, for example, optionally substituted aziridinyl, diaziridinyl, oxiranyl, oxaziridinyl, oxetanyl, thiiranyl, thietanyl, pyrrolidinyl, piperazinyl, morpholinyl, piperidinyl, tetrahydrofuranyl, tetrahydropyranyl, 1,4-dioxanyl, hexahydroazepinyl, oxepanyl, 1,2-oxathlolanyl or oxazolidinyl,

C₃-C₇-heterocycloalkenyl radical such as, for example, optionally substituted azirinyl, diazirinyl, thiirenyl, thietyl, pyrrolinyls, oxazolinyls, azepinyl, oxepinyl, α-pyranyl, β-pyranyl, γ-pyranyl, dihydropyranyls, 2,5-dihydropyrrolinyl or 4,5-dihydrooxazolyl,

a branched or unbranched, optionally substituted C₁-C₄-alkylene-C₃-C₇-cycloalkyl radical which is composed, for example, of branched or unbranched C₁-C₄-alkylene radicals such as, for example, methylene, ethylene, propylene, n-butylene, isobutylene or t-butylene and, for example, the aforementioned C₃-C₇-cycloalkyl radicals,

a branched or unbranched, optionally substituted C₁-C₄-alkylene-C₃-C₇-heterocycloalkyl or C₁-C₄-alkylene-C₃-C₇-heterocycloalkenyl radical, which are composed of optionally substituted C₁-C₄-alkylene radicals such as, for example, methylene, ethylene, propylene, n-butylene, isobutylene or t-butylene and, for example, the aforementioned C₃-C₇-heterocycloalkyl or C₃-C₇-heterocycloalkenyl radicals, preferred radicals being those containing in the cyclic moiety one or two heteroatoms selected from the group of N, O or S and up to two double bonds,

an optionally substituted

aryl radical, preferably optionally substituted phenyl, 1-naphthyl or 2-naphthyl,

arylalkyl radical, preferably optionally substituted benzyl or phenethyl,

hetaryl radical, preferably optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3 isoxazolyl, 4-isoxazolyl, 5-isoxazolyl, thiadiazolyl, oxadiazolyl or triazinyl, or their fused derivatives such as, for example, indazolyl, indolyl, benzothienyl, benzofuranyl, indolinyl, benzimidazolyl, benzothiazolyl, benzoxazolyl, quinolinyl or isoquinolinyl,

hetarylalkyl radical, preferably optionally substituted —CH₂-2-pyridyl, —CH₂-3-pyridyl, —CH₂-4-pyridyl, —CH₂-2-thienyl, —CH₂-3-thienyl, —CH₂-2-thiazolyl, —CH₂-4-thiazolyl, CH₂-5-thiazolyl, —CH₂-CH₂-2-pyridyl, —CH₂-CH₂-3-pyridyl, —CH₂—CH₂-4-pyridyl, —CH₂—CH₂-2-thienyl, —CH₂—CH₂-3-thienyl, —CH₂—CH₂-2-thiazolyl, —CH₂—CH₂-4-thiazolyl or —CH₂—CH₂-5-thiazolyl or

a radical —S—R_{G}⁴, —O—R_{G}⁴, —SO—R_{G}⁴, —CO—OR_{G}⁴, —O—CO—NR_{G}⁵R_{G}⁶, —SO₂—NR_{G}⁵R_{G}⁶, —CO—NR_{G}⁵R_{G}⁶, —NR_{G}⁵R_{G}⁶, CO—R_{G}⁴.

It is also possible for the R_{G}¹ and R_{G}² radicals together to form an optionally substituted, saturated, unsaturated or aromatic 3- to 9-membered carbocycle, polycarbocycle, heterocycle or polyheterocycle which may contain up to 4 heteroatoms selected from the group of O, N, S.

Preferred radicals for R_{G}¹ in the structural element G are hydrogen, a branched or unbranched, optionally substituted C₁-C₆-alkyl radical, preferably CF₃, C₂-C₆-alkenyl, C₂-C₆-alkynyl radical, C₁-C₄-alkylene-OR_{G}⁴, optionally substituted aryl, arylalkyl, hetaryl or hetarylalkyl or a radical —O—R_{G}⁴.

Preferred radicals for R_{G}² in the structural element G are hydrogen, CN, halogen, a branched or unbranched, optionally substituted C₁-C₆-alkyl radical, preferably CF₃, an optionally substituted C₃-C₇-cycloalkyl, aryl, arylalkyl, hetaryl or hetarylalkyl radical, an —SO—R_{G}⁴, —SO₂—R_{G}⁴, —CO—OR_{G}⁴, —SO₂—NR_{G}⁵R_{G}⁶, —CO—NR_{G}⁵R_{G}⁶, —NR_{G}⁶, —NR_{G}⁵R_{G}⁶, CO—R_{G}⁴, C₁-C₄-alkylene-CO—OR_{G}⁴, C₁-C₄-alkylene-SO₂—NR_{G}⁵R_{G}⁶, C₁-C₄-alkylene-CO—NR_{G}⁵R_{G}⁶ or C₁-C₄-alkylene-NR_{G}⁵R_{G}⁸ radical or an optionally substituted C₁-C₄-alkylene-C₃-C₇-cycloalkyl, C₃-C₇-heterocycloalkyl, C₃-C₇-heterocycloalkenyl, C₁-C₄-alkylene-C₃-C₇-heterocycloalkyl or C₁-C₄-alkylene-C₃-C₇-heterocycloalkenyl radical, the radicals preferred for the last four radicals being those containing in the cyclic moiety one or two heteroatoms selected from the group of N, O or S and up to two double bonds.

R_{G}² in the structural element G is particularly preferably a branched or unbranched, optionally substituted C₁-C₆-alkyl radical, an optionally substituted C₃-C₇-cycloalkyl, aryl, arylalkyl, hetaryl or hetarylalkyl radical, a —CO—OR_{G}⁴, —CO—NR_{G}⁵R_{G}⁶, —NR_{G}⁵R_{G}⁶, C₁-C₄-alkylene-CO—NR_{G}⁵R_{G}⁶ or C₁-C₄-alkylene-NR_{G}⁵R_{G}⁶ or an optionally substituted C₃-C₇-heterocycloalkyl, C₃-C₇-heterocycloalkenyl, C₁-C₄-alkylene-C₃-C₇-heterocycloalkyl or C₁-C₄-alkylene-C₃-C₇-heterocycloalkenyl radical, the radicals preferred for the last four radicals being those containing in the cyclic moiety one or two heteroatoms selected from the group of N, O or S and up to two double bonds.

R_{G}³ is hydrogen, a hydroxyl group, CN, a branched or unbranched, optionally substituted

C₁-C₆-alkyl radical as described, for example, for R_{G}¹ above,

C₁-C₄-alkoxy radical as described, for example, for RL¹¹ above,

an optionally substituted C₃-C₇-cycloalkyl, aryl or arylalkyl radical as in each case described, for example, for R_{G}¹ above, or:

an optionally substituted —O—C₃-C₇-cycloalkyl radical, —O-aryl or —O-alkylene-aryl radical which is composed, for example, in each case of the group —O— and the corresponding radicals described above for R_{G}¹.

A branched or unbranched, optionally substituted C₁-C₈-alkyl radical for R_{G}⁴, R_{G}^{4*}, R_{G}⁵ and R_{G}⁶ means, independently of one another, for example the C₁-C₆-alkyl radicals mentioned above for R_{G}¹ plus the radicals heptyl and octyl.

Preferred substituents of the branched or unbranched, optionally substituted C₁-C₈-alkyl radicals for R_{G}⁴, R_{G}⁴, R_{G}⁵ and R_{G}⁶ are, independently of one another, the radicals halogen, hydroxyl, C₁-C₄-alkoxy, —CN, —COOH and —CO—O-C₁-C₄-alkyl.

A branched or unbranched, optionally substituted C₂-C₆alkenyl, C₂₋C₆-alkynyl or C₁-C₄-alkylene-C₃-C₇-cycloakyl radical, an optionally substituted C₃-C₇-cycloalkyl, aryl, arylalkyl, hetaryl or hetarylalkyl radical for R_{G}⁴, R_{G}^{4*}, R_{G}⁵ and R_{G}⁸ means, independently of one another, for example the corresponding radicals mentioned above for R_{G}¹.

Preferred branched or unbranched, optionally substituted -C₁-C₅-alkylene-C₁-C₄-alkoxy radicals for R_{G}⁴, R_{G}⁴, R_{G}⁵ und R_{G}⁶ are, independently of one another, methoxymethylene, ethoxymethylene, t-butoxymethylene, methoxyethylene or ethoxyethylene.

Preferred branched or unbranched, optionally substituted mono- and bisalkylaminoalkylene or acylaminoalkylene radicals for R_{G}⁴, R_{G}^{4*}, R_{G}⁵ and R_{G}⁶ are, independently of one another, branched or unbranched, optionally substituted -C₁-C₄-alkylene-NH(C₁-C₄-alkyl), -C₁-C₄-alkylene-N(C₁-C₄-alkyl)₂ and -C₁-C₄-alkylene-NH—CO-C₁-C₄-alkyl radicals.

Preferred optionally substituted heterocycloalkyl, heterocycloalkenyl, C₁-C₄-alkylene-heterocycloalkyl or C₁-C₄-alkylene-heterocycloalkenyl radicals for R_{G}⁴, R_{G}^{4*}, R_{G}⁵ and R_{G}⁶ are, independently of one another, the C₃-C₇-heterocycloalkyl, C₃-C₇-heterocycloalkenyl, C₁-C₄-alkylene-C₃-C₇-heterocycloalkyl or C₁-C₄-alkylene-C₃-C₇-heterocycloalkenyl radicals described above for R_{G}¹.

Particularly preferred optionally substituted heterocycloalkyl, heterocycloalkenyl, C₁-C₄-alkylene-heterocycloalkyl or C₁-C₄-alkylene-heterocycloalkenyl radicals for R_{G}⁴, R_{G}⁴, R_{G}⁵ and R_{G}⁶ are, independently of one another, the C₃-C₇-heterocycloalkyl, C₃-C₇-heterocycloalkenyl, C₁-C₄-alkylene-C₃-C₇-heterocycloalkyl or C₁-C₄-alkylene-C₃-C₇-heterocycloalkenyl radicals described above for R_{G}¹ the cyclic moiety containing one or two heteroatoms selected from the group of N, O or S and up to two double bonds.

It is also possible for R_{G}⁵ and R_{G}⁸ independently of one another to be an —SO₂—R_{G}⁴, —CO—O—R_{G}⁴, —CO—NR_{G}⁴R_{G}^{4*} or —CO—R_{G}⁴ radical, where R_{G}^{4*} gives an R_{G}⁴ radical independent of R_{G}⁴.

Preferred structural elements G are composed of at least one preferred radical of the radicals belonging to structural element G, or the preferred substitution pattern of structural element G, while the remaining radicals may vary widely.

Particularly preferred structural elements G are composed of the preferred radicals of the structural element G.

Very particularly preferred structural elements G are composed of the preferred radicals of the structural element G and the preferred substitution pattern of the structural element G.

In a very particularly preferred embodiment of the structural element G, the substitution pattern is as specified in formula I_{GB}, ZG is oxygen. R_{G}¹ is hydrogen, R_{G}² is methyl and the structural element G is incorporated in such a way that the structural element E is connected to the carbon in position 4 and the structural element L is connect to the nitrogen in position 1.

Structural element B means a structural element containing at least one atom which can, under physiological conditions, form hydrogen bonds as hydrogen acceptor, where the distance between at least one hydrogen acceptor atom and structural element G along the shortest possible route along the structural element framework is from 4 to 13 atomic linkages. The arrangement of the structural framework of structural element B may vary widely.

Examples of suitable atoms which, under physiological conditions, are able to form hydrogen bonds as hydrogen acceptors are atoms with Lewis base properties such as, for example, the heteroatoms nitrogen, oxygen or sulfur.

Physiological conditions mean a pH prevailing at the site in an organism at which the ligands Interact with the receptors. In the present case, the pH under physiological conditions is, for example, from 5 to 9.

In a preferred embodiment, the structural element B is a structural element of the formula IE

A-E-I₈

where A and E have the following meanings:

A a structural element selected from the group:

a 4- to 8-membered monocyclic saturated, unsaturated or aromatic hydrocarbon which may contain up to 4 heteroatoms selected from the group of O, N or S, it being possible in each case, independently of one another, for the ring nitrogen which is present where appropriate or the carbons to be substituted, with the proviso that at least one heteroatom selected from the group of O, N or S is present in the structural element A, a 9- to 14-membered polycyclic saturated, unsaturated or aromatic hydrocarbon which may contain up to 6 heteroatoms selected from the group of N, O or S, it being possible in each case, independently of one another, for the ring nitrogen which is present where appropriate or the carbons to be substituted, with the proviso that at least one heteroatom selected from the group of O, N or S is present in the structural element A, a radical

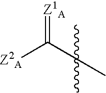

where

Z_{A}¹ Is oxygen, sulfur or optionally substituted nitrogen, preferably oxygen or nitrogen and

Z_{A}² is optionally substituted nitrogen, oxygen or sulfur, preferably nitrogen,

or a radical

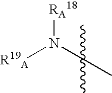

where

R_{A}¹⁸, R_{A}¹⁹

are, independently of one another, hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-heterocycloalkyl, C₁-C₄-alkylene-heterocycloalkenyl or hetarylalkyl radical, or an —SO₂—R_{G}⁴, —CO—OR_{G}⁴, —CO—NR_{G}⁴R_{G}* or —CO—R_{G}⁴ radical,

and

E a spacer structural element which covalently connects structural element A to structural element G, where the number of atomic linkages along the shortest possible route along the structural element framework E is from 3 to 12.

In a particularly preferred embodiment, the structural element A is a structural element selected from the group of structural elements of the formulae I_{A}¹ to I_{A}¹⁹,

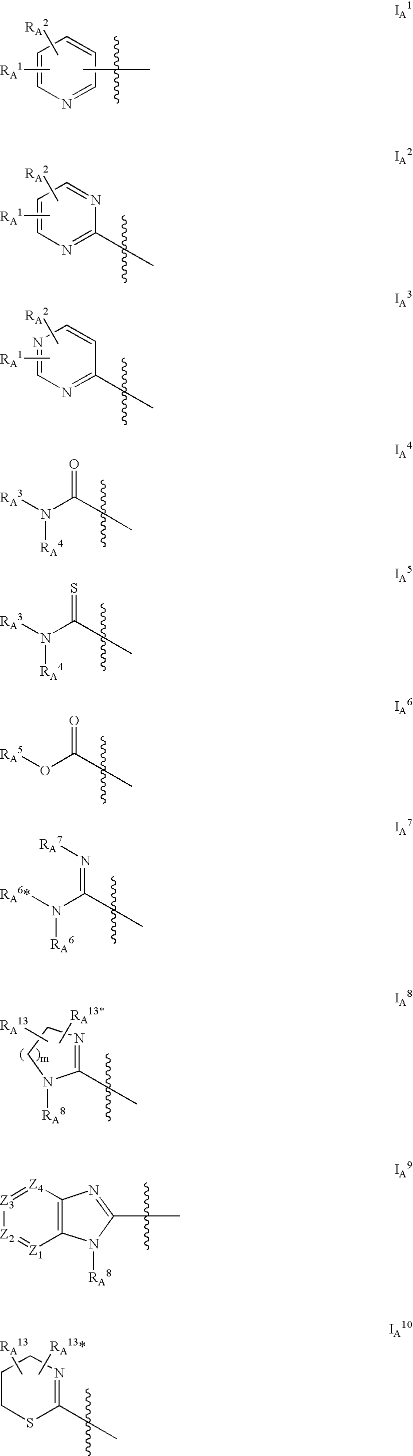

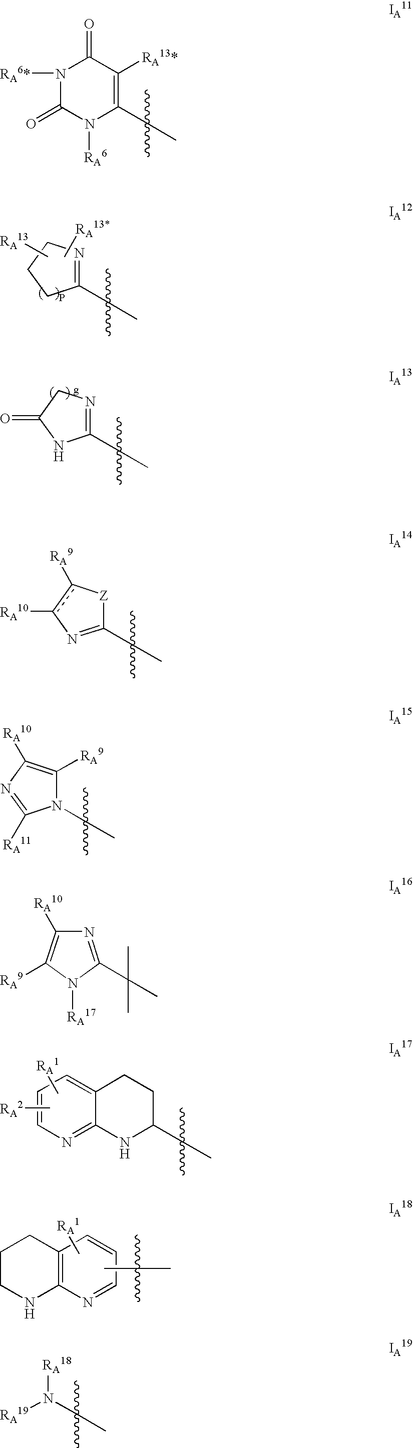

where

m, p, q

are, independently of one another, 1, 2 or 3,

R_{A}¹, R_{A}²

are, independently of one another, hydrogen, CN, halogen, a branched or unbranched, optionally substituted C₁-C₆-alkyl or CO-C₁-C₆-alkyl radical or an optionally substituted aryl, arylalkyl, hetaryl, hetarylalkyl or C₃-C₇-cycloalkyl radical or a CO—O—R_{A}¹⁴, O—R_{A}¹⁴, S—A¹⁴, NR_{A}¹⁵R_{A}¹⁶, CO—NR_{A}¹⁵R_{A}¹⁶ or SO₂NR_{A}¹⁵R_{A}¹⁶ radical or the two radicals R_{A}¹ and R_{A}² together are a fused, optionally substituted, 5- or 6-membered, unsaturated or aromatic carbocyclic or heterocyclic system which may contain up to three heteroatoms selected from the group of O, N or S.

R_{A}¹³, R_{A}^{13*}

are, independently of one another, hydrogen, CN, halogen, a branched or unbranched, optionally substituted C₁-C₆-alkyl radical or an optionally substituted aryl, arylalkyl, hetaryl, C₃-C₇-cycloalkyl radical or a CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}R_{A}¹⁶ radical,

where

R_{A}¹⁴ is hydrogen, a branched or unbranched, optionally substituted C₁-C₆-alkyl, alkylene-C₁-C₄-alkoxy, C₂-C₆-alkenyl, C₂-C₆-alkynyl or C₁-C₆-alkylene-C₃-C₇-cycloalkyl radical or an optionally substituted C₃-C₇-cycloalkyl, aryl, arylalkyl, hetaryl or hetarylalkyl radical,

R_{A}¹⁵, R_{A}¹⁶,

are, independently of one another, hydrogen, a branched or unbranched, optionally substituted C₁-C₆-alkyl, CO—C₁-C₆-alkyl, SO₂-C₁-C₆-alkyl, COO—C₁-C₆-alkyl, CO—NH-C₁-C₈-alkyl, arylalkyl, COO-alkylene-aryl, SO₂-alkylene-aryl, CO—NH-alkylene-aryl, CO—NH-alkylene-hetaryl or hetarylalkyl radical or an optionally substituted C₃-C₇-cycloalkyl, aryl, CO-aryl, CO—NH-aryl, SO₂-aryl, hetaryl, CO—NH-hetaryl, or CO-hetaryl radical,

R_{A}^{3,} R_{A}⁴

Are, independently of one another, hydrogen, —(CH₂)ₙ—(X_{A})ⱼ—R_{A}¹², or the two radicals together are a 3- to 8-membered, saturated, unsaturated or aromatic N heterocyclic system which may additionally contain two other, identical or different heteroatoms O, N or S, it being possible for the cyclic system optionally to be substituted, or for another, optionally substituted, saturated, unsaturated or aromatic cyclic system to be fused to this cyclic system,

where

n is 0, 1, 2 or 3,

j is 0 or 1,

X_{A} is —CO—, —CO—N(R_{X}¹)—, —N(R_{X}¹)—CO—, N(R_{X}¹)—CO—N(R_{X}¹*)—, —N(R_{X}¹)—CO—O—, —O—, —S—, —SO₂—N(R_{X}¹)—, —SO₂—O—, —CO—O—, —O—CO—, —O—CO—N(R_{X}¹)—, —N(R_{X}¹)— or —N(R_{X}¹)—SO₂—,

R_{A}¹² is hydrogen, a branched or unbranched, optionally substituted C₁-C₆-alkyl radical, an optionally C₁-C₄-alkyl- or aryl-substituted C₂-C₆-alkynyl or C₂-C₆-alkenyl radical or a 3- to 6-membered, saturated or unsaturated heterocyclic system which is substituted by up to three Identical or different radicals and which may contain up to three different or identical heteroatoms O, N, S, or C₃-C₇-cycloalkyl, aryl or heteroaryl radical, it being possible for two radicals together to be a fused, saturated, unsaturated or aromatic carbocyclic or heterocyclic system which may contain up to three different or identical heteroatoms O, N, S, and it being possible for the cyclic system optionally to be substituted or for another, optionally substituted, saturated, unsaturated or aromatic cyclic system to be fused to this cyclic system, or the R_{A}¹² radical forms together with R_{X}¹ or R_{X}¹* a saturated or unsaturated C₃-C₇-heterocycle which may optionally contain up to two other heteroatoms selected from the group of O, S or N,

R_{X}^{1,} R_{X}¹*

are, Independently of one another, hydrogen, a branched or unbranched, optionally substituted C₁-C₆alkyl, C₁-C₆-alkoxyalkyl, C₂-C₆-alkenyl, C₂C₁₂-alkynyl, CO-C₁C₆-alkyl, CO—O-C₁-C₆-alkyl or SO₂-C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂-alkylene-aryl radical,

R_{A}⁵ is a branched or unbranched, optionally substituted C₁-C₆-alkyl, arylalky, C₃-C₇-cycloalkyl or C₁-C₄-alkylene-C₃-C₇-cycloalkyl radical or an optionally substituted aryl, hetaryl, heterocycloalkyl or heterocycloalkenyl radical,

R_{A}⁸, R_{A}^{6*}

are hydrogen, a branched or unbranched, optionally substituted C₁-C₄-alkyl, —CO—O-C₁-C₄-alkyl, arylalkyl, —CO—O-alkylene-aryl, —CO—O-allyl, —CO-C₁-C₄-alkyl, —CO-alkylene-aryl, C₃-C₇-cycloalkyl or —CO-allyl radical or the two radicals R_{A}⁶ and R_{A}^{6*} in the structural element I_{A}⁷ together are an optionally substituted, saturated, unsaturated or aromatic heterocyclic system which, in addition to the ring nitrogen, may contain up to two other different or identical heteroatoms O, N, S,

R_{A}⁷ is hydrogen, —OH, —CN, —CONH₂, a branched or unbranched, optionally substituted C₁-C₄-alkyl, C₁-C₄-alkoxy, C₃-C₇-cycloalkyl or —O—CO-C₁-C₄-alkyl radical or an optionally substituted arylalkyl, —O-alkylene-aryl, —O—CO-aryl, —O—CO-alkylene-aryl or —O—CO-allyl radical, or the two radicals R_{A}⁶ and R_{A}⁷ together are an optionally substituted, unsaturated or aromatic heterocyclic system which, in addition to the ring nitrogen, may contain up to two other different or identical heteroatoms O, N, S,

R_{A}⁸ is hydrogen, a branched or unbranched, optionally substituted C₁-C₄-alkyl, CO-C₁-C₄-alkyl, SO₂-C₁-C₄-alkyl or CO—O-C₁-C₄ alkyl radical or an optionally substituted aryl, CO-aryl, SO-aryl, CO—O-aryl, CO-alkylene-aryl, SO₂-alkylene-aryl, CO—O-alkylene-aryl or alkylene-aryl radical,

R_{A}⁹, R_{A}¹⁰

are, independently of one another, hydrogen, —CN, halogen, a branched or unbranched, optionally substituted C₁-C₆-alkyl radical or an optionally substituted aryl, arylalkyl, hetaryl, C₃-C₇-cycloalkyl radical or a CO—O—R_{A}¹⁴, O—R_{A}¹⁴, S—R_{A}¹⁴, NR_{A}¹⁵R_{A}¹⁶, SO₂—NR_{A}¹⁵R_{A}¹⁰ or CO—NR_{A}¹⁵R_{A}¹⁶ radical, or the two radicals R_{A}⁹ and R_{A}¹⁰ in the structural element IA¹⁴ together are a 5- to 7-membered saturated, unsaturated or aromatic carbocyclic or heterocyclic system which may contain up to three different or identical heteroatoms O, N, S and is optionally substituted by up to three identical or different radicals,

R_{A}¹¹ Is hydrogen, —CN, halogen, a branched or unbranched, optionally substituted C₁-C₅-alkyl radical or an optionally substituted aryl, arylalkyl, hetaryl, C₃-C₇-cycloalkyl radical or a CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—R_{A}¹⁵R_{A}¹⁶ radical,

R_{A}¹⁷ Is hydrogen or the two radicals R_{A}⁹ and R_{A}¹⁷ in the structural element I_{A}¹⁶ together are a 5- to 7-membered saturated, unsaturated or aromatic heterocycle which, in addition to the ring nitrogen, may contain up to three different or identical heteroatoms O, N, S and is optionally substituted by up to three identical or different radicals,

R_{A}¹⁸, R_{A}¹⁹

are, independently of one another, hydrogen, a branched or unbranched, optionally substituted C₁-C₈-alkyl, C₂-C₆-alkenyl, C₂C₆-alkynyl-, C₁-C₅-alkylene-C₁-C₄-alkoxy, mono- and bisalkylaminoalkylene or acylamino-alkylene radical or an optionally substituted aryl, heterocycloalkyl, heterocycloalkenyl, hetaryl, C₃-C₇-cycloalkyl, C₁-C₄-alkylenene-C₃-C₇-cycloalkyl, arylalkyl, C₁-C₄-alkylene-heterocycloalkyl, C₁-C₄alkylene-heterocycloalkenyl or hetarylalkyl radical, or an —SO₂—R_{G}⁴; —CO—OR_{G}⁴, —CO—NR_{G}⁴R_{G}⁴* or —CO—R_{G}⁴ radical,

Z^{1,} Z^{2,} Z^{3,} Z⁴

are, independently of one another, nitrogen, C—H, C-halogen or a branched or unbranched, optionally substituted C-C₁-C₄-alkyl or C-C₁-C₄-alkoxy radical,

Z⁵ is NR_{A}⁸, oxygen or sulfur.

In a further, very particularly preferred embodiment, the structural element A is a structural element of the formulae I_{A}¹, I_{A}⁴, I_{A}⁷, I_{A}⁸I_{A}⁹ or I_{A}¹⁸.

A branched or unbranched, optionally substituted C₁-C₆-alkyl radical for R_{A}¹ or R_{A}² means, independently of one another, for example the corresponding radicals described above for R_{G}¹ preferably methyl or trifluoromethyl.

The branched or unbranched, optionally substituted CO—C₁-C₆-alkyl radical for R_{A}¹ or R_{A}² in the structural elements I_{A}¹, I_{A}², I_{A}³ or I_{A}¹⁷ is composed, for example, of the CO group and the branched or unbranched, optionally substituted C₁-C₆-alkyl radicals described above for R_{A}¹ or R_{A}².

Optionally substituted hetaryl, hetarylalkyl, aryl, arylalkyl or C₃-C₇-cycloalkyl radicals for R_{A}¹ or R_{A}² mean, independently of one another, for example the corresponding radicals described above for R_{G}¹.

The optionally substituted CO—O—R_{A}¹⁴, O—R_{A}¹⁴, S—R_{A}¹⁴, NR_{A}¹⁵R_{A}¹⁶, CC—NR_{A}¹⁶R_{A}¹⁸ or SO₂NR_{A}¹⁵R_{A}¹⁶ radicals for RA¹ or RA² are composed, for example, of the groups CO—O, O, S, N, CO—N or SO₂—N and the radicals R_{A}¹⁴, R_{A}¹⁵ and R_{A}¹⁶ which are described in detail below.

It is also possible for the two radicals R_{A}¹ and R_{A}² together to form a fused, optionally substituted, 5- or 6-membered, unsaturated or aromatic carbocyclic or heterocycilc system which may contain up to three heteroatoms selected from the group of O, N or S.

R_{A}¹³ and R_{A}¹³* are, independently of one another, hydrogen, CN,

halogen such as, for example, fluorine, chlorine, bromine or iodine,

a branched or unbranched, optionally substituted C₁-C₆-alkyl radical, for example as described above for R_{G}¹, preferably methyl or trifluoromethyl or

an optionally substituted aryl, arylalkyl, hetaryl or C₃-C₇-cycloalkyl radical or a CO—O—R_{A}¹⁴, O—R_{A}¹⁴, NR_{A}¹⁴, NR_{A}¹⁵R_{A}¹⁶, SO₂NR_{A}¹⁵R_{A}¹⁶ or CO—NR_{A}¹⁵R_{A}¹⁸ radical as in each case described above for R_{A}¹.

Preferred radicals for R_{A}¹³ and R_{A}¹³* are the radicals hydrogen, F, Cl or a branched or unbranched, optionally substituted C₁-C₆-alkyl radical, optionally substituted aryl or arylalkyl or a CO—O—R_{A}¹⁴, O—R_{A}¹⁴, NR_{A}¹⁵R_{A}¹⁶, SO₂—NR_{A}¹⁵R_{A}¹⁶ or CO—NR_{A}¹⁵R_{A}¹⁶ radical.

A branched or unbranched, optionally substituted C₁-C₆-alkyl, C₃-C₇-cycloalkyl, alkylene-cycloalkyl, alkylene-C₁-C₄-alkoxy, C₂-C₆-alkenyl or C₂-C₆-alkynyl radical for R_{A}¹⁴ in structural element A mean, for example, the corresponding radicals described above for R_{G}¹.

Optionally substituted aryl, arylalkyl, hetaryl or alkylhetaryl radicals for R_{A}¹⁴ in structural element A mean, for example, the corresponding radicals described above for R_{G}¹.

Preferred radicals for R_{A}¹⁴ are hydrogen, a branched or unbranched, optionally substituted C₁-C₆-alkyl radical and optionally substituted benzyl.

A branched or unbranched, optionally substituted C₁-C₆-alkyl or arylalkyl radical or an optionally substituted C₅-C₇-cycloalkyl, aryl, hetaryl or hetarylalkyl radical for R_{A}¹⁵ or R_{A}¹⁶ mean, independently of one another, for example the corresponding radicals described above for R_{A}¹⁴.

The branched or unbranched, optionally substituted CO-C₁-C₆-alkyl, SO₂-C₁-C₆-alkyl, COO—C₁-C₆-alkyl, CO—NH-C₁-C₆-alkylene-aryl, COO-alkylene-aryl, CO—NH-alkylene-aryl, —CO—NH— alkylene-hetaryl or SO₂-alkylene-aryl radicals or the optionally substituted CO-aryl, SO₂-aryl, CO—NH-aryl, CO—NH-hetaryl or CO-hetaryl radicals for R_{A}¹⁵ or R_{A}¹⁶ are composed, for example, of the appropriate groups —CO—, —SO₂—, —CO—O—, —CO—NH— and the appropriate branched or unbranched, optionally substituted C₁-C₆-alkyl, hetarylalkyl or arylalkyl radicals described above or the appropriate optionally substituted aryl or hetaryl radicals.

A radical —(CH₂)ₙ—(X_{A})ⱼ—R_{A}² for R_{A}³ or R_{A}⁴ means, independently of one another, a radical which is composed of the appropriate —(CH₂)ₙ—, (X_{A})ⱼ and R_{A}¹² radicals. In these cases, n can be 0, 1, 2 or 3 and j can be 0 or 1.

X_{A} is a doubly bonded radical selected from the group of —CO—, —CO—N(R_{X}¹)—, —N(R_{X}¹)—CO—, —N(R_{X}¹)—CO—, N(R_{X}¹*)—, —N(R_{X}¹)—CO—O—, —O—, —S—, —SO₂—, —SO₂—N(R_{X}¹)—, —SO₂—O—, —CO—O—, —O—CO—, —O—CO—N(R_{X}¹)—, —N(R_{X}¹)— or —N(R_{X}¹)—SO₂—.

R_{A}¹² is hydrogen,

a branched or unbranched, optionally substituted C₁₋₄-alkyl radical as described for R_{G}¹,

an optionally C₁-C₄-alkyl- or aryl-substituted C₂-C₆-alkynyl or C₂-C₆-alkenyl radical, for example as described above for R_{L}⁹,

or A 3- to 6-membered, saturated or unsaturated heterocycle which is substituted by up to three identical or different radicals and which may contain up to three different or identical heteroatoms O, N, S, such as, for example, optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 2-(1,3,4)-thiadiazolyl, 2-(1,3,4)-oxadiazolyl, 3-isoxazolyl, 4-isoxazolyl, 5-isoxazolyl, triazinyl.

It is also possible for R_{A}¹² and R_{X}¹ or R_{X}¹* together to form a saturated or unsaturated C₃-C₇-heterocycle which may optionally contain up to two other heteroatoms selected from the group O, S or N.

The R_{A}¹² radical preferably forms together with the R_{X}¹ or R_{C}¹* radical a cyclic amine as C₃-C₇-heterocycle in the case where the radicals are bonded to the same nitrogen atom, such as, for example, N-pyrrolidinyl, N-piperidinyl, N-hexahydroazepinyl, N-morpholinyl or N-piperazinyl, it being possible for the free amine protons on heterocycles having free amine protons, such as, for example, N-piperazinyl, to be replaced by conventional amine protective groups such as, for example, methyl, benzyl, Boc (tert-butoxycarbonyl), Z (benzyloxycarbonyl), tosyl, —SO₂-C₁-C₄-alkyl, —SO₂-phenyl or —SO₂-benzyl.

A branched or unbranched, optionally substituted C₁-C₆-alkyl, C₁₋₆-alkoxyalkyl, C₂-C₆-alkenyl, C₂-C₁₂-alkynyl, CO—C₁-C₆-alkyl, CO—O-C₁-C₆-alkyl or SO₂-C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂-alkylene-aryl radical for R_{X}¹ and R_{X}¹* mean, independently of one another, for example the radicals described above for R_{L}¹⁴ and R_{L}¹⁴*.

Preferred radicals for R_{X}¹ and R_{X}¹* are, independently of one another, hydrogen, methyl, cyclopropyl, allyl and propargyl.

R_{A}³ and R_{A}⁴ may also together form a 3- to 8-membered, saturated, unsaturated or aromatic N heterocycle which may additionally contain two other identical or different heteroatoms O, N or S, it being possible for the cyclic system optionally to be substituted or for another, optionally substituted, saturated, unsaturated or aromatic cyclic system to be fused to this cyclic system,

R_{A}⁵ is a branched or unbranched, optionally substituted C₁-C₆-alkyl, arylalkyl, C₁-C₄-alkyl-C₃-C₇-cycloalkyl or C₃-C₇-cycloalkyl radical or an optionally substituted aryl, hetaryl, heterocycloalkyl or heterocycloalkenyl radical, for example as described above for R_{G}₄, R_{G}⁵ and R_{G}⁶.

R_{A}⁶ and R_{A}^{6*} are, independently of one another, hydrogen, a branched or unbranched, optionally substituted

—CO—O-C₁-C₄-alkyl radical such as, for example, optionally substituted methyl, ethyl, propyl, 1-methylethyl, butyl, 1-methylpropyl, 2-methylpropyl or 1,1-dimethylethyl,

—CO—O-C₁-C₄-alkyl or —CO-C₁-C₄-alkyl radical such as, for example, composed of the group —CO—O— or and the C₁-C₄-alkyl radicals described above,

arylalkyl radical as described above for R_{G}¹,

—CO—O-alkylene-aryl- or —CO-alkylene-aryl radical such as, for example, composed of the group —CO—O— or —CO— and the arylalkyl radicals described above,

—CO—O-allyl or allyl radical,

or C₃-C₇-cycloalkyl radical, for example as described above for R_{G}¹.

It is also possible for the two radicals R_{A}⁶ and R_{A}^{6*} in structural element I_{A}⁷ together to form an optionally substituted, saturated, unsaturated or aromatic heterocycle which, in addition to the ring nitrogen, may contain up to two other different or identical heteroatoms O, N, S.

R_{A}⁷ is hydrogen, —OH, —CN —CONH_{2,} a branched or unbranched, optionally substituted C₁-C₄-alkyl radical, for example as described above for R_{A}⁶, C₁-C₄-alkoxy, arylalkyl or C₃-C₇-cycloalkyl radical, for example as described above for R_{L}¹⁴, a branched or unbranched, optionally substituted —O—CO-C₁-C₄-alkyl radical which is composed of the group —O—CO— and, for example, of the C₁-C₄-alkyl radicals described above, or an optionally substituted —O-alkylene-aryl, —O—CO-aryl, —O—CO-alkylene-aryl or —O—CO-allyl radical which is composed of the groups —O— or —O—CO— and, for example, of the corresponding radicals described above for R_{G}¹.

It is also possible for the two radicals R_{A}⁶ and R_{A}⁷ together to form an optionally substituted, unsaturated or aromatic heterocycle which, in addition to the ring nitrogen, may contain up to two other different or identical heteroatoms O, N, S.

A branched or unbranched, optionally substituted C₁-C₄alkyl radical or an optionally substituted aryl or arylalkyl radical for R_{A}⁸ in structural element A means, for example, the corresponding radicals described above for R_{A}¹⁵, where the CO—C₁-C₄-alkyl, SO₂₋C₁-C₄-alkyl, CO—O—C₁-C₄-alkyl, CO-aryl, SO₂-aryl, CO—O-aryl, CO-alkylene-aryl, SO₂₋C₁-C₄-alkylene-aryl or CO-alkylene-aryl radicals are composed, in analogy to the other radicals, of the groups CO, SO₂ or COO and, for example, of the corresponding C₁-C₄-alkyl, aryl or arylalkyl radicals described above for R_{A}¹⁵, and these radicals may optionally be substituted.

A branched or unbranched, optionally substituted C₁-C₆-alkyl radical or an optionally substituted aryl, arylalkyl, hetaryl or C₃-C₇-cycloalkyl radical in each case for R_{A}⁹ or R_{A}¹⁰ means, independently of one another, for example, the corresponding radicals described above for R_{A}¹⁴, preferably methyl or trifluoromethyl.

A CO—O—R_{A}¹⁴, O—R_{A}¹⁴, S—R_{A}¹⁴, SO₂—NR_{A}¹⁵R_{A}¹⁶, NR_{A}¹⁵R_{A}¹⁶ or CO—NR_{A}¹⁵R_{A}¹⁶ radical means in each case for R_{A}⁹ or R_{A}¹⁰, independently of one another, for example, the corresponding radicals described above for R_{A}¹³.

It is also possible for the two radicals R_{A}⁹ and R_{A}¹⁰ together in the structural element I_{A}¹⁴ to form a 5- to 7-membered saturated, unsaturated or aromatic carbocyclic or heterocyclic system which may contain up to three different or identical heteroatoms O, N, S and is optionally substituted by up to three identical or different radicals.

Substituents mean in this case in particular halogen, CN, a branched or unbranched, optionally substituted C₁-C₄-alkyl radical such as, for example, methyl or trifluoromethyl or the radicals O—RA¹⁴, S—R_{A}¹⁴, NR_{A}¹⁵R_{A}¹⁸, CO—NR_{A}¹⁵R_{A}¹⁰ or ((R_{A}⁸)HN)C═N—R_{A}⁷.

A branched or unbranched, optionally substituted C₁-C₆-alkyl radical or an optionally substituted aryl, arylalkyl, hetaryl, C₃-C₇ cycloalkyl radical or a CO—O—R_{A}¹⁴, O—R_{A}¹⁴, S—R_{A}¹⁴, NR_{A}¹⁵R_{A}¹⁶, SO₂-NR_{A}¹⁵R_{A}¹⁸ or CO—NR_{A}¹⁵R_{A}¹⁶ for R_{A}¹¹ means, for example, the corresponding radicals described above for R_{A}⁹.

It is also possible for the two radicals R_{A}⁹ and R_{A}¹⁷ In the structural element I_{A}¹⁶ together to form a 5- to 7-membered saturated, unsaturated or aromatic heterocycle which, in addition to the ring nitrogen, may contain up to three different or identical heteroatoms O, N, S and is optionally substituted by up to three identical or different radicals.

A branched or unbranched, optionally substituted C₁-C₈-alkyl, C₂-C₆-alkenyl, C₂-C₆-alkynyl, C₁-C₅-alkylene-C₁-C₄-alkoxy, mono- and bisalkylaminoalkylene or acylamino-alkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-heterocycloalkyl, C₁-C₄-alkylene-heterocycloalkenyl or hetarylalkyl radical or an —SO₂R_{G}⁴, —CO—OR_{G}⁴, —CO—NR_{G}⁴R_{G}⁴* or —COR—R_{G}⁴ radical for R_{A}¹⁸ and R_{A}¹⁹ means, independently of one another, for example the radicals described above for R_{G}^{5,} preferably hydrogen or a branched or unbranched, optionally substituted C₁-C₈-alkyl radical.

Z^{1,} Z^{2,} Z^{3,} Z⁴ are, independently of one another, nitrogen, C—H, C-halogen such as, for example, C—F, C—Cl, C—Br or C—I or a branched or unbranched, optionally substituted C-C₁-C₄-alkyl radical which is composed of a carbon radical and, for example, a C₁-C₄-alkyl radical described above for R_{A}⁶, or a branched or unbranched, optionally substituted C-C₁-C₄-alkoxy radical which is composed of a carbon radical and, for example, a C₁-₄-alkoxy radical described above for R_{A}⁷.

Z⁵ is oxygen, sulfur or an NR_{A}⁸ radical.

Preferred structural elements A are composed of at least one preferred radical of the radicals belonging to structural element A, while the remaining radicals may vary widely.

Particularly preferred structural elements A are composed of the preferred radicals of the structural element A.

In a preferred embodiment, the spacer structural element E means a structural element which consists of a branched or unbranched, optionally substituted and heteroatom-containing aliphatic C₂-C₃₀-hydrocarbon radical and/or of a 4- to 20-membered, optionally substituted and heteroatom-containing, aliphatic or aromatic mono- or polycyclic hydrocarbon radical.

In a particularly preferred embodiment, the spacer structural element E is a structural element of the formula I_{E}

—(NR_{E}¹)ⱼ-E₁-(U_{E})ₕ— I_{E}

where

U_{E} is oxygen, sulfur or NR_{E}²,

h is 0 or 1,

i is 0 or 1,

R_{E}¹, R_{E}²

are, independently of one another, hydrogen, a branched or branched, optionally substituted C₁C₆-alkyl, C₁-C₆-alkoxyalkyl, C₂-C₆-alkenyl, C₂-C₁₂-alkynyl, CO-C₁-C₆-alkyl, CO—O-C₁-C₆-alkyl, CO—NH-C₁-C₆-alkoxyalkyl, CO—NH-C₁-C₆-alkyl or SO₂-C₁-C₆-alkyl radical or an optionally substituted hetaryl, arylalkyl, C₃-C₇-cycloalkyl, CO—O-alkylene-aryl, CO—NH-alkylene-aryl, CO-alkylene-aryl, CO-aryl, CO—NH-aryl, SO₂-aryl, CO-hetaryl, SO₂-alkylene-aryl, SO₂-hetaryl or SO₂-alkylene-hetaryl radical,

E₁ is a structural element of the formula I_{E1}

—(CR_{E}³R_{E}⁴)ₖ₁-(L_{E})ₖ₂—(CR_{E}⁵R_{E}⁵)ₖ₃-(Q_{E})ₖ₄—(CR_{E}⁷R_{E}⁸)ₖ₅—(T_{E})ₖ₅—(CR_{E}⁹R¹⁰)_{k7—} I_{E1}

where

k2, k4, k6

are 0 or 1,

k1, k3, k5, k7

are 0, 1 or 2,

R_{E}³, R_{E}⁴, R_{E}⁵, R_{E}⁶, R_{E}⁷, R_{E}⁸, R_{E}⁹, R_{E}¹⁰

are, independently of one another, hydrogen, halogen, a hydroxyl group, a branched or unbranched, optionally substituted C₁-C₆-alkyl, C₂-C₆-alkenyl, C₂-C₆-alkynyl or alkylene-cycloalkyl radical, a —(CH₂)ₓ—(Y_{E})_{z}R_{E}¹¹ radical, an optionally substituted C₃-C₇-cycloalkyl, aryl, arylalkyl, hetaryl or hetarylalkyl radical or, independently of one another, in each case two radicals R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbo- or heterocyclic system which may contain up to three heteroatoms from the group of O, N or S,

x is 0, 1, 2, 3 or 4,

z is 0 or 1,

Y_{E} is —CO—, —CO—N(R_{y}²)—, —N(R_{y}²)—CO—, —N(R_{y}²)—CO—N(R_{y}²*)—, —N(R_{y}²)—CO—O—, —O—, —S—, —SO₂—N(R_{y}²)—, —SO₂—O—, —CO—O—, —O—CO—, —O—CO—N(R_{y}²⁾—, —N(R_{y}²)— or —N(R_{y}²)—SO₂—,

R_{y}^{2,} R_{y}²*

are, independently of one another, hydrogen, a branched or unbranched, optionally substituted C₁-C₆-alkyl, C₂-C₆-alkynyl, C₂-C₆-alkenyl, CO-C₁-C₆-alkyl, CO—O-C₁-C₆-alkyl or SO₂-C₁-C₆-alkyl radical or an optionally substituted hetaryl, hetarylalkyl, arylalkyl, C₃-C₇-cycloalkyl, CO—O-alkylene-aryl, CO-alkylene-aryl, CO-aryl, SO₂-aryl, CO-hetaryl or SO₂-alkylene-aryl radical,

RE¹¹ is hydrogen, a hydroxyl group, CN, halogen, a branched or unbranched, optionally substituted C₁-C₆-alkyl radical, an optionally substituted C₃-C₄-cycloalkyl, aryl, -hetaryl or arylalkyl radical, an optionally C₁-C₄-alkyl- or aryl-substituted C₂-C₆-alkynyl or C₂-C₆-alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₉-alkylene cycloalkyl radical, or a 3- to 8-membered, saturated or unsaturated heterocyclic system which is substituted by up to three Identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system optionally to be substituted or for another, optionally substituted, saturated, unsaturated or aromatic cyclic system to be fused to this cyclic system, or the R_{E}¹¹ radical forms together with R_{Y}² or R_{Y}²* a saturated or unsaturated C₃-C₇-heterocyclic system which may optionally contain up to two other heteroatoms selected from the group of O, S or N,

L_{E}, T_{E}

are, independently of one another, CO, CO—NR_{E}¹², NR_{E}¹²—O, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴—C(═CR_{E}¹³R_{E}¹⁴), CR_{E}¹³═CR_{E}¹⁴, —CR_{E}¹³(OR_{E}¹⁵)—CHR_{E}¹⁴—, —CHR_{E}¹³—CR_{E}¹⁴(OR_{E}¹⁵)—,

R_{E}¹² is hydrogen, a branched or unbranched, optionally substituted C₁-C₈alkyl, C₂-C₆-alkenyl, C₂-C₈-alkynyl, an optionally substituted C₃-C₇-cycloalkyl, hetaryl, arylalkyl or hetarylalkyl radical or a CO—R_{E}¹⁸, COOR_{E}¹⁶ or SO₂—R_{E}¹⁶ radical,

R_{E}¹³, R_{E}¹⁴

are, independently of one another, hydrogen, a hydroxyl group, a branched or unbranched, optionally substituted C₁-C₆-alkyl, C₁-C₄-alkoxy, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,

R_{E}¹⁵ is hydrogen, a branched or unbranched, optionally substituted C₁-C₆-alkyl, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,

R_{E}¹⁶ is hydrogen, a hydroxyl group, a branched or unbranched, optionally substituted C₁-C₈-alkyl, C₂-C₆-alkenyl, C₂-C₆-alkynyl or C₁-C₅alkylene C₁-C₄-alkoxy radical, or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical and

Q_{E} is an optionally substituted 4- to 11-membered mono- or polycyclic aliphatic or aromatic hydrocarbon which may contain up to 6 double bonds and up to 6 identical or different heteroatoms selected from the group N, O or S, it being possible for the ring carbons or ring nitrogens optionally to be substituted.

U_{E} in structural element E is oxygen, sulfur or NR_{E}², with sulfur or NR_{E}² being preferred and NR_{E}² being particularly preferred.

The coefficients h and i are, independently of one another, 0 or 1.

In a preferred embodiment, the coefficient i is 1.

A branched or unbranched, optionally substituted C₁-C₆-alkyl, C₁-C₆-alkoxyalkyl, C₂-C₆-alkenyl, C₂-C₁₂-alkynyl or arylalkyl radical or an optionally substituted aryl, hetaryl or C₃-C₇-cycloalkyl for R_{E}¹ and R_{E}² in structural element E means, independently of one another, for example the corresponding radicals described above for R_{L}¹⁴.

The branched or unbranched, optionally substituted CO-C₁-C₆-alkyl, CO—O—C₁-C₆-alkyl, CO—NH-C₁-C₆-alkoxyalkyl, CO—NH—C₁-C₆-alkyl or SO₂₁-C₁-C₆-alkyl radicals or the optionally substituted CO—O-alkylene-aryl, CO—NH-alkylene-aryl, CO-alkylene-aryl, CO-aryl, CO—NH-aryl, SO₂-aryl, CO-hetaryl, SO₂-alkylene-aryl, SO₂-hetaryl or SO₂-alkylene-hetaryl radicals for R_{E}¹ and R_{E}² are composed, independently of one another, for example of the appropriate groups CO, COO, CONH or SO₂ and the appropriate radicals mentioned above.

Preferred radicals for R_{E}¹ or R_{E}² are, independently of one another, hydrogen, a branched or unbranched, optionally substituted C₁-C₆-alkyl, C₁-C₆-alkoxy, C₂-C₆-alkenyl, C₂-C₁₂-alkynyl or arylalkyl radical, or an optionally substituted hetaryl or C₃-C₇-cycloalkyl radical.

Particularly preferred radicals for R_{E}¹ or R_{E}² are hydrogen, methyl, cyclopropyl, allyl or propargyl.

E₁ means a structural element of the formula I_{E1}

—(CR_{E}³R_{E}⁴)ₖ₁-(L_{E})ₖ₂-(CR_{E}⁵SR_{E}⁶)ₖ₃-(Q_{E})ₖ₄—(CR_{E}⁷R_{E}⁸)ₖ₅—(T_{E})ₖ₆—(CR_{E}⁹R_{E}¹⁰)ₖ₇— I_{E1}

where the coefficients

k2, k4 or k6 can be 0 or 1 and k1, k3, k5 or k7 can be 0, 1 or 2.

A branched or unbranched, optionally substituted C₁-C₆-alkyl, C₂-C₅-alkenyl, C₂-C₆-alkynyl or alkylene-cycloalkyl radical or an optionally substituted C₃-C₇-cycloalkyl, aryl, arylalkyl, hetaryl or hetarylalkyl radical for R_{E}³, R_{E}⁴, R_{E}⁵, R_{E}⁶, R_{E}⁷, R_{E}⁸, R_{E}⁹ or R_{E}¹⁰ mean, independently of one another, for example the corresponding radicals mentioned above for R_{L}¹.

It is also possible in each case independently of one another for two radicals R_{E}³ and R_{E}⁴ or R_{E}⁵ and R_{E}⁸ or R_{E}⁷ and R_{E}⁸ or R_{E}⁹ and R_{E}¹⁰ together to form a 3- to 7-membered, optionally substituted, saturated or unsaturated carbo- or heterocyclic system which may contain up to three heteroatoms from the group of O, N or S.

The —(CH₂)_{z}—(Y_{E})₂—R_{E}¹¹ radical is composed of a C₀-C₄-alkylene radical, optionally a linking element Y_{E} selected from the group of —CO—, —CO—N(R_{y}²)—, —N(R_{y}²)—CO—, —N(R_{y}²)—CO—N(R_{y}²*)—, —N(R_{y}²)—CO—O—, —O—, —S—, —SO₂—, —SO₂—N(R_{y}²)—, —SO₂—O—, —CO—O—, —O—CO—, —O—CO—N(R_{y}²⁾—, —N(R_{y}²) or —N(R_{y}²)—SO₂—, preferably selected from the group of —CO—N(R_{y}²)—, —N(R_{y}²)—CO—O—, —O—, —SO₂—N(R_{y}²)—, —N(R_{y}²)— or —N(R_{y}²)—SO₂—, and the radical R_{E}¹¹, where

R_{y}² and R²*

are, independently of one another, hydrogen, a branched or unbranched, optionally substituted C₁-C₆-alkyl, C₂-C₆-alkenyl, CO-C₁-C₆-alkynyl, CO—O-C₁-C₆-alkyl or SO₂-C₁-C₆-alkyl radical or an optionally substituted hetaryl, hetarylalkyl, arylalkyl, C₃-C₇-cycloalkyl, CO—O-alkylene-aryl, CO-alkylene-aryl, CO-aryl, SO₂-aryl, CO-hetaryl or SO₂-alkylene-aryl radical, preferably independently of one another hydrogen, methyl, cyclopropyl, allyl, propargyl, and

R_{E}¹¹

is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, an optionally C₁-C₄alkyl- or aryl-substituted C₂-C₆-alkynyl or C₂-C₆-alkenyl radical, an optionally substituted C₆-C₁₂-bicycloalkyl, C₂-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system optionally to be substituted or for another, optionally substituted, saturated, unsaturated or aromatic cyclic system to be fused to this cyclic system, such as, for example, optionally substituted 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 2-imidazolyl, 4-imidazolyl, 5-imidazolyl, 3-pyridazinyl, 4-pyridazinyl, 5-pyridazinyl, 6-pyridazinyl, 2-(1,3,4)-thiadiazolyl, 2-(1,3,4)-oxadiazolyl, 3-isoxazolyl, 4-isoxazolyl, 5-isoxazolyl or triazinyl.

It is also possible for R_{E}¹¹ and R_{Y}² or R_{Y}²* together to form a saturated or unsaturated C₃-C₇-heterocycle which may optionally contain up to two other heteroatoms selected from the group of O, S or N.

The R_{E}¹¹ and R_{Y}² or R_{Y}²* radicals preferably together form a cyclic amine as C₃-C₇-heterocycle in the case where the radicals are bonded to the same nitrogen atom, such as, for example, N-pyrrolidinyl, N-piperidinyl, N-hexahydroazepinyl, N-morpholinyl or N-piperazinyl, it being possible for the free amine protons on heterocycles having free amine protons, such as, for example, N-piperazinyl, to be replaced by conventional amine protective groups such as, for example, methyl, benzyl, Boc (tert-butoxycarbonyl), Z (benzyloxycarbonyl), tosyl, —SO₂-C₁-C₄-alkyl, —SO₂-phenyl or —SO₂-enzyl.

Preferred radicals for R_{E}³, R_{E}⁴, R_{E}⁵, R_{E}⁶, R_{E}⁷, R_{E}⁸, R_{E}⁹ or R_{E}¹⁰ are, independently of one another, hydrogen, a branched or unbranched, optionally substituted C₁-C₆alkyl radical, optionally substituted aryl or the —(CH₂)ₓ—(Y_{E})ₓ—R_{E}¹¹ radical.

In a preferred embodiment of the structural element E₁, one radical of R_{E}³ and R_{E}⁴ or R_{E}⁵ and R_{E}⁶ or R_{E}⁷ and R_{E}⁸ or R_{E}⁹ and R_{E}¹⁰ is, independently of one another, hydrogen or methyl.

In a particularly preferred embodiment of the structural element E₁, the R_{E}³, R_{E}⁴, R_{E}⁵, R_{E}⁶, R_{E}⁷, R_{E}⁸, R_{E}⁹ or R_{E}¹⁰ radicals are, independently of one another, hydrogen or methyl.

L_{E} and T_{E} are, independently of one another, CO, CO—NR_{E}¹² NR_{E}¹²—CO, sulfur, SO, SO₂, SO₂—NR_{E}¹², NR_{E}¹²—SO₂, CS, CS—NR_{E}², NR_{E}¹²S, CS—O, O—CS, CO—O, O—CO, oxygen, ethynylene, CR_{E}¹³—CR_{E}¹⁴, C(═CR_{E}¹³R_{E}¹⁴), CR_{E}¹³═CR_{E}¹⁴, —CR_{E}(OR_{E}¹⁵)——CHR_{E}¹⁴— or —CHR_{E}¹³—CR_{E}¹⁴, (OR_{E}¹⁵)—, preferably CO—NR_{E}¹², NR_{E}¹²—CO, SO_NRE¹2, NRE¹²—SO₂ and oxygen.

R_{E}¹² is hydrogen, a branched or unbranched, optionally substituted C₁-C₆-alkyl, C₂-C₆-alkenyl or C₂-C₆-alkynyl radical or an optionally substituted C₃-C₇-cycloalkyl, hetaryl, arylalkyl or hetarylalkyl radical, for example as described above in each case for R_{L}¹, or a CO—R_{E}¹⁶, COOR_{E}¹⁶ or SO₂—R_{E}¹⁶ radical, preferably hydrogen, methyl, allyl, propargyl and cyclopropyl.

A branched or unbranched, optionally substituted C₁-C₆-alkyl, C₂C₆-alkenyl or C₂-C₆-alkynyl radical or an optionally substituted C₃-C₇-cycloalkyl, aryl, arylalkyl, hetaryl or hetarylalkyl radical for R_{E}¹³, R_{E}¹⁴ or R_{E}¹⁵ means, independently of one another, for example the corresponding radicals described above for R_{L}¹.

A branched or unbranched, optionally substituted C₁-C₄-alkoxy radical for R_{E}¹³ or R_{E}¹⁴ means, independently of one another, for example the C₁-C₄-alkoxy radicals described above for R_{A}¹⁴.

Preferred alkylene-cycloalkyl radicals for R_{E}¹³, R_{E}¹⁴ or R_{E}¹⁵ are, independently of one another, for example the C₁-C₄-alkylene-C₃-C₇-cyloalkyl radicals described above for R_{L}¹.

A branched or unbranched, optionally substituted C₁-C₆-alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkyl, C₁-C₄-alkylene cycloalkyl, arylalkyl, C₁-C₄-alkylene-C₃-C₇-heterocycloalkyl, C₁-C₄-alkylene-C₃-C₇-heterocycloalkenyl or hetarylalkyl radical for R_{E}¹⁸ means, for example, the corresponding radicals described above for R_{G}⁴.

An optionally substituted 4- to 11-membered mono- or polycyclic aliphatic or aromatic hydrocarbon which may contain up to 6 double bonds and up to 6 identical or different heteroatoms selected from the group of N, O, S, it being possible for the ring carbons or ring nitrogens optionally to be substituted, for Q_{E} preferably means optionally substituted arylene such as, for example; optionally substituted phenylene or naphthylene, optionally substituted hetarylene such as, for example, the radicals

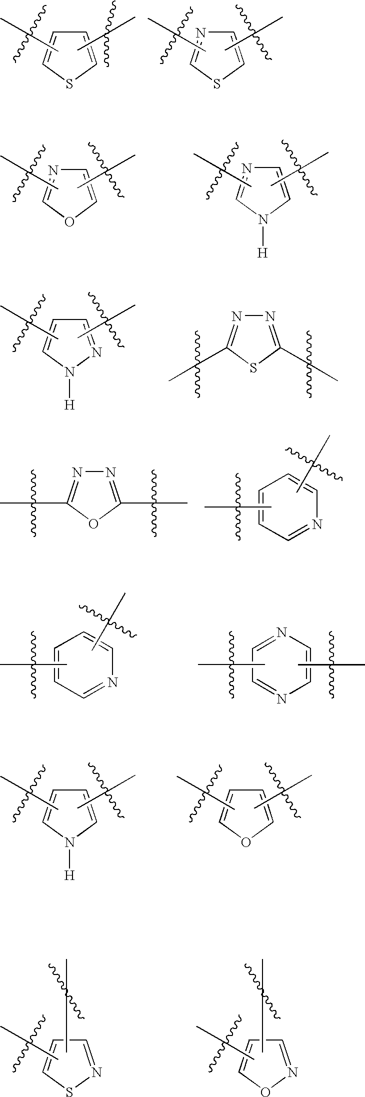

and their substituted or fused derivatives, or radicals of the formulae I_{E}¹ to I_{E}¹¹.

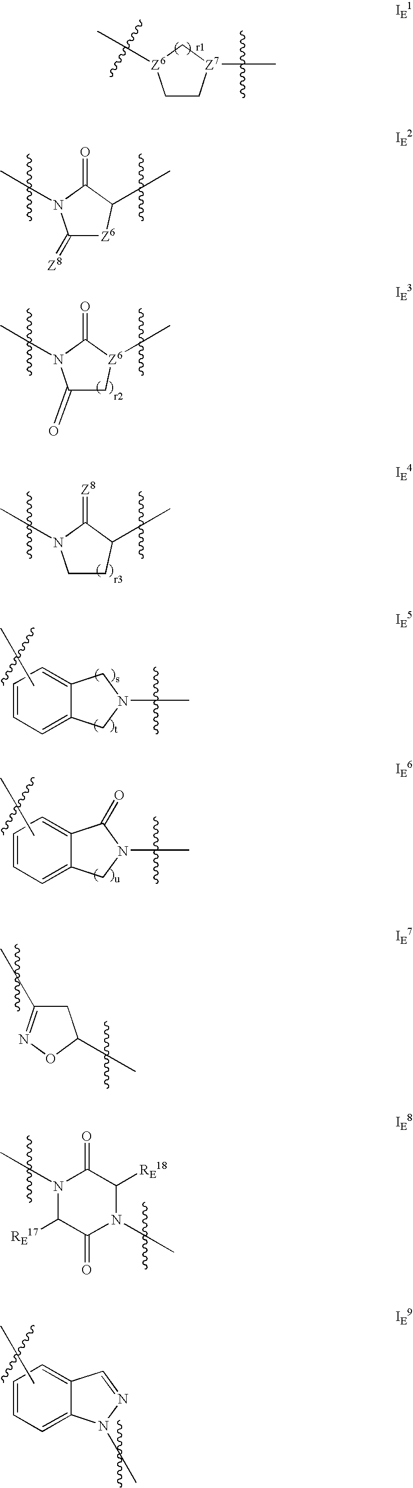

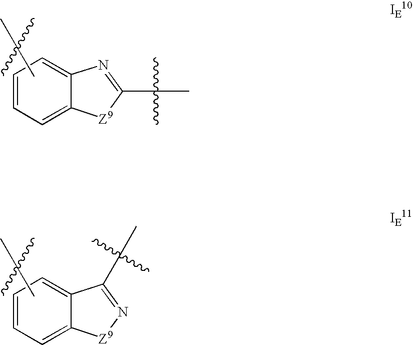

it being possible for the radicals to be incorporated in both orientations.

Z⁶ and Z⁷ are, independently of one another, CH or nitrogen.

Z is oxygen, sulfur or NH

Z⁹ is oxygen, sulfur or NR_{E}¹⁹.

r1, r2, r3 and t are, independently of one another, 0, 1, 2 or 3.

s and u are, independently of one another, 0, 1 or 2.

Q_{E} is particularly preferably optionally substituted phenylene, a radical

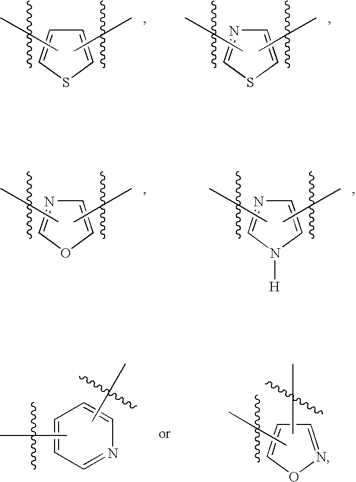

and their substituted or fused derivatives, or radicals of the formulae I_{E}¹, I^{E}², I^{E}³, I^{E}⁴ and I^{E}⁷, it being possible for the radicals to be incorporated in both orientations.

R_{E}¹⁷ and R_{E}¹⁸ are, independently of one another, hydrogen, —NO₂, —NH₂—, —CN, —COOH, a hydroxyl group, halogen, a branched or unbranched, optionally substituted C₁-C₆-alkyl, C₁-C₄-alkoxy, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical as described above in each case.

R_{E}¹⁹ is, independently of one another, hydrogen, a branched or unbranched, optionally substituted C₁-C₆-alkyl, C₁-C₆-alkoxyalkyl, CO—O-C₁-C₆-alkynyl, CO—C₁-C₆-alkyl, CO—O-C₁-C₆-alkyl or SO₂-C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂-alkylene-aryl radical, preferably hydrogen or a branched or unbranched, optionally substituted C₁-C₆-alkyl radical.

Preferred structural elements E are composed of at least one preferred radical of the radicals belonging to the structural element E, while the remaining radicals may vary widely.

Particularly preferred structural elements E are composed of the preferred radicals of the structural element E.

Preferred structural elements B are composed either of the preferred structural element A, while E may vary widely, or of the preferred structural element E, while A may vary widely.

The compounds of the formula I and the intermediates for preparing them may have one or more asymmetric substituted carbon atoms. The compounds may be in the form of pure enantiomers or pure diastereomers or a mixture thereof. The use of an enantiomerically pure compound as active substance is preferred.

The compounds of the formula I may also exist in other tautomeric forms.

The compounds of the formula I may also be in the form of physiologically tolerated salts.

The compounds of the formula I may also be in a form as prodrugs in which the compounds of the formula I are liberated under physiological conditions. Reference may be made here by way of example to group T in the structural element L, which in some cases contains groups Which can be hydrolyzed under physiological conditions to a free carboxyl group. Derivatized structural elements B and A which release the structural element B or A under physiological conditions are also suitable.

In preferred compounds of the formula I, in each case one of the three structural elements B, G or L has the preferred range, while the remaining structural elements may vary widely.

In particularly preferred compounds of the formula 1, in each case two of the three structural elements B, G or L have the preferred range, while the remaining structural elements may vary widely.

In very particularly preferred compounds of the formula I, in each case all three structural elements B, G or L have the preferred range, while the remaining structural element may vary widely.

Preferred compounds of the formula I have, for example, the preferred structural element L, while the structural elements B and G may vary widely.

In particularly preferred compounds of the formula I, for example B is replaced by the structural element A-E-, and the compounds have, for example, the preferred structural element L and the preferred structural element G, while the structural elements E and A may vary widely.

Further particularly preferred compounds have the preferred structural elements E, G and L, while the structural elements A may vary widely.

Further very particularly preferred compounds have the preferred structural elements A, G and L, while the structural elements E may vary widely.

Further very particularly preferred compounds have the preferred structural elements A, E, G and L.

The compounds of the formula I and the starting materials used to prepare them can be prepared generally by methods of organic chemistry known to the skilled worker, as described in standard works such as, for example, Houben-Weyl, “Methoden der Organischen Chemie”, Thieme-Verlag, Stuttgart, or March “Advanced Organic Chemistry”, 4th Edition, Wiley & Sons. Further preparation methods are also described in R. Larock, “Comprehensive Organic Transformations”, Weinheim 1989, in particular the preparation of alkenes, alkynes, halides, amines, ethers, alcohols, phenols, aldehydes, ketones, nitriles, carboxylic acids, esters, amides and acid chlorides. The selection of suitable protective groups for functional groups, and the introduction or elimination of the protective groups are described, for example, in Greene and Wuts in “Protective Groups in Organic Synthesis”, 2 Edition, Wiley & Sons, 1991.

Synthesis of compounds of the formula I can be carried out either in solution or on a polymeric support, the reaction conditions used in each case being those known and suitable for the particular reactions. It is moreover possible to make use of variants which are known per se but which are not mentioned here.

The general synthesis of compounds of type I where, as described above, A-E- may be the structural element B- and

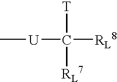

may be the structural element is described in Schemes 1-7. Unless indicated otherwise, all the starting materials and reagents can be purchased or can be prepared by conventional methods from precursors which can be purchased.

Compounds of the general formula I are synthesized, for example, starting from appropriately substituted 4-thioxo-3,4-dihydro-pyrimidin-2(1-ones of the general formula II as intermediate. 4-Thioxo-3,4-dihydropyrimidin-2(1H)-ones of type II are known and can be prepared by known methods as described, for example, in Katritzky and Rees, “Comprehensive Heterocyclic Chemistry”, Pergamon Press, volume 3; pp. 135139 and the literature quoted therein.

A preferred method for synthesizing 4-thioxo-3,4-dihydropyrimidin-2(1H)-ones comprises, for example, addition of enamines onto isothiocyanates with subsequent cyclization as described by Goerdeler et al. in Chem. Ber. 1963, pp. 526-533, and Chem. Ber. 1965, pp. 1531-1542. 4Thioxo-3,4-dihydropyrimidin-2(1H)-ones can particularly preferably be prepared by the method described by Lamon in J. Heterocycl. Chem. 1968, 5, 837-844, which is based on the reaction of an enamine with alkoxy- or aryloxycarbonyl isothiocyanate. Compounds of the formula I can be synthesized by reacting appropriate enamine derivatives of the general formula III in which X is preferably a morpholine, pyrrolidine or piperidine residue with primary amines to form the subs. 4-thioxo-3,4-dihydropyrimidin-2(1H)-ones II (Scheme 1).

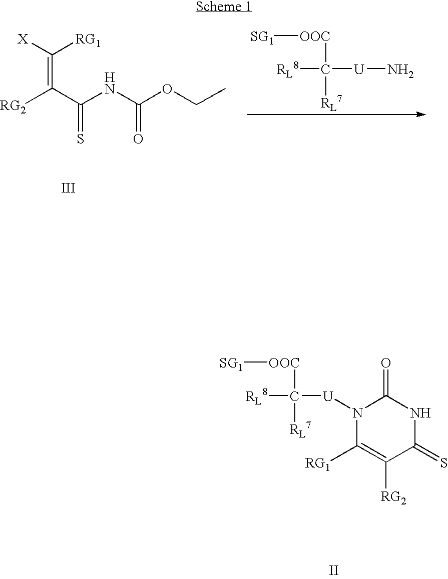

SG₁ is a protective group for the carboxyl function, or the SG₁OOC— radical is T, as described above.

It is particularly efficient to carry out the synthesis on solid phase for example, by using the carboxyl function as anchor group for attachment to a solid support (SG₁=solid support). Methods for solid-phase synthesis are described in detail, for example, by Bunin in “The Combinatorial Index” (Academic Press, 1998). In the case where U contains another functional group or the side chain of an amino acid which contains a so-called side-chain functionality, this is advantageously protected by suitable protective groups.

For further reaction, the 4-thioxo group in compounds of the general formula II is alkylated by standard methods with addition of a base. It is possible to use as base an alkali metal or alkaline earth metal hydride such as sodium hydride, potassium hydride or calcium hydride, a carbonate such as alkali metal carbonate, for example sodium or potassium carbonate, an alkali metal or alkaline earth metal hydroxide such as sodium or potassium hydroxide, an alcoholate such as, for example, sodium methanolate, potassium tert-butanolate, an organometallic compound such as butyllithium or alkali metal amides such as lithium diisdpropylamide, lithium, sodium or potassium bis(trimethylsilyl)amide, tertiary amines such as triethylamine, 1,8-diaza-bicyclo[5.4.0]undec-7-ene or ethyl diisopropylamine. The use of alkali metal carbonates such as Cs₂CO₃ or tertiary amines such as ethyl diisopropylamine is particularly preferred.

In the case where the U_{E} radical in compounds of the general formula I is oxygen or NR_{E}², or in the case where h=0, U_{E} is absent and thus there is a direct linkage between the fragments A-E and G via an N group present in the fragment, the 4-thioxo group is preferably converted into the corresponding thiocyanate by alkylation with cyanogen bromide, as described, for example, in Tetrahedron Letters 1991, 32 (22), 2505-2508 (Scheme 2). The thiocyanate of the formula IVa can then be reacted with suitable amines or alcohols of the general formula A-E-(U_{E})ₕ—H (V) by methods known to the skilled worker, possibly with addition of a base, to give the compounds of the general formula VI (Scheme 2). In this scheme, for illustration, -E′- represents the spacer structural element E without the linker (U_{E})ₕ.

In the case where the U_{E} radical in compounds of the formula I is sulfur, it is possible to use as alkylating agent directly a compound of the general formula A-E-Y (VII), in which case the group Y is a conventional leaving group such as, for example, halogen such as chlorine, bromine, iodine or aryl or alkylsulfonyl, which is optionally substituted by halogen, alkyl or haloalkyl, such as, for example, toluenesulfonyl, trifluoromethanesulfonyl and methylsulfonyl or another equivalent leaving group (Scheme 2).

Another preferred method for preparing compounds of the general formula I with U_{E}=sulfur is the conversion of compounds of the general formula II into the corresponding sulfanylacetonitriles (IVb), which can then be reacted with thiols of the structure A-E-SH (Vb) to give compounds VI.

Elimination of the protective group SG₁ under standard conditions (see below) leads to compounds of the general formula I. In the case where SG₁ is C₁-C₄-alkyl or benzyl or the case where SG₁—OOC— is T, the compounds of the general formula VI correspond directly to the compounds of type I.

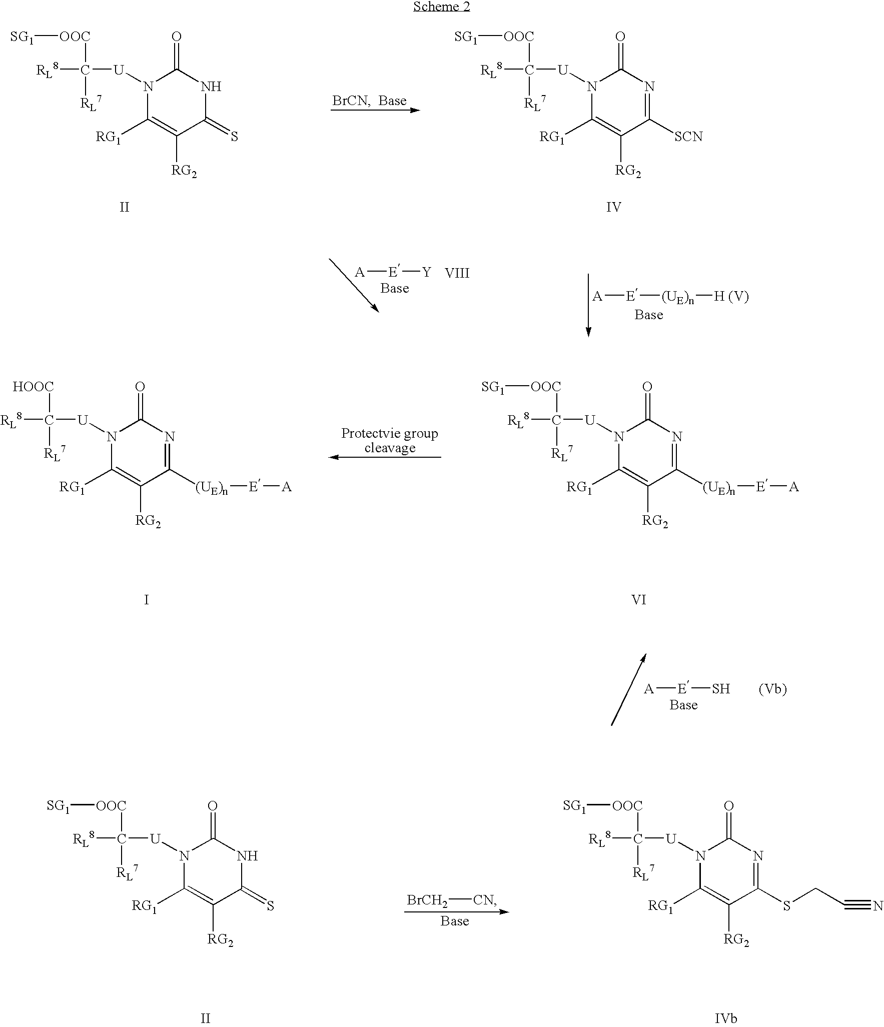

It is possible to use as protective groups SG all protective groups which are in use and are known to the skilled worker from peptide synthesis, and are also described in the standard works such as, for example, Bodanszky “The Practice of Peptide Synthesis”, 2^{nd} Edition, Springer-Verlag 1994, and Bodanszky “Principles of Peptide Synthesis”, Springer-Verlag 1984. Elimination of the protective groups in the compounds of the formula VI, and the protective groups used for preparing the compounds V and VII, likewise takes place under conditions known to the skilled worker and described, for 1 example, by Greene and Wuts in “Protective Groups in Organic Synthesis”, 2^{nd} Edition, Wiley & Sons, 1991.

The amino protective groups preferably used are Boo, Fmoc, benzyloxycarbonyl (Z), acetyl, trityl or Mtr. The acid protective groups used, such as, for example, SG₁, are preferably C₁-C₄-alkyl such as, for example, methyl, ethyl, tert-butyl or else benzyl or trityl, or else polymer-bound protective groups in the form of the commercially available polystyrene resins such as, for example, 2-chlorotrityl chloride-resin or Wang resin (supplied by Bachem or Novabiochem).

Elimination of acid-labile protective groups (for example Boc, tert-butyl, Mtr, trityl) can be effected, depending on the protective group used, with organic acids such as, for example, trifluoroacetic acid (TFA), trichloroacetic acid, perchloric acid, trifluoroethanol, sulfonic acids such as, for example, benzene- or p-toluenesulfonic acid or else inorganic acids such as, for example, hydrochloric acid or sulfuric acid, the acids generally being employed in excess.

In the case of trityl, the addition of thiols such as, for example, thioanisole or thiophenol may be advantageous. The presence of an additional inert solvent is possible but not always necessary. Suitable and preferred inert solvents are organic solvents, for example carboxylic acids such as acetic acid, ethers such as THF or dioxane, amides such as DMF or dimethylacetamide, halogenated hydrocarbons such as dichloromethane, alcohols such as methanol, isopropanol or water. Mixtures of said solvents are also suitable. The reaction temperature for these reactions is between 10° C. and 50° C., preferably in a range between 0° C. and 30° C.

Base-labile protective groups such as Fmoc are cleaved by treatment with organic amines such as, for example, dimethylamine, diethylamine, morpholine, piperidine, as 5-50% solutions in CH₂Cl₂ or DMF. The reaction temperature for these reactions is between 10° C. and 50° C., preferably in a range between 0° C. and 30° C.

Acid protective groups such as methyl or ethyl are preferably cleaved by basic hydrolysis in an inert solvent. The bases preferably used are alkali metal or alkaline earth metal hydroxides, preferably NaOH, KOH or LiOH. The solvents used are all conventional inert solvents such as, for example, hydrocarbons such as hexane, heptane, petroleum ether, toluene, benzene or xylene, chlorinated hydrocarbons such as trichloroethylene, 1,2-dichloroethane, tetrachloromethane, chloroform, dichloromethane, alcohols such as methanol, ethanol, isopropanol, n-propanol, n-butanol or tert-butanol, ethers such as diethyl ether, methyl tert-butyl ether, diisopropyl ether, tetrahydrofuran, dioxane, glycol ethers such as ethylene glycol monomethyl ether or monoethyl ether, ethylene glycol dimethyl ether, ketones such as acetone, butanone, amides such as dimethylformamide (DMF), dimethylacetamide or acetamide, nitriles such as acetonitrile, sulfoxides such as dimethyl sulfoxide, sulfolane, N-methylpyrrolidone, 1,3-dimethyltetrahydro-2(1H)-pyrimidinone (DMPU), 1,3-dimethyl-2-imidazolidinone, nitro compounds such as nitromethane or nitrobenzene, water , or mixtures of said solvents. The addition of a phase-transfer catalyst may be advantageous, depending on the solvent or mixture of solvents used. The reaction temperature for these reactions is generally between −10° C. and 100° C.

Protective groups which can be eliminated by hydrogenolysis, such as benzyloxycarbonyl (Z) or benzyl, can be eliminated, for example, by hydrogenolysis in the presence of a catalyst (for example a noble metal catalyst on activated carbon as support). Suitable solvents are those indicated above, and in particular alcohols such as methanol or ethanol, amides such as DMF or dimethylacetamide, esters such as, for example, ethyl acetate. The hydrogenolysis is ordinarily carried out under a pressure of 1-200 bar and at temperatures between 0° C. and 100° C.; the addition of an acid such as, for example, acetic acid or hydrochloric acid may be advantageous. 5-10% Pd on activated carbon is preferably used as catalyst.

Building blocks of type E are generally assembled by methods known to the skilled worker. The building blocks used can be either purchased or obtained by methods known from the literature. The synthesis of some of these building blocks is described by way of example in the examples section.

In the case where the fragments Q_{E} present in compounds of type V and VII are a hetaryl radical, the building blocks used can either be purchased or obtained by methods known to the skilled worker. A large number of preparation methods are described in detail in Houben-Weyl's “Methoden der organischen Chemie” (volume E6: furans, thiophenes, pyrroles, indoles, benzothiophenes, -furans, -pyrroles; volume E7: quinolines, pyridines, volume E8: isoxazoles, oxazoles, thiazoles, pyrazoles, imidazoles and their benzo-fused representatives, and oxadiazoles, thiadiazoles and triazoles; volume E9: pyridazines, pyrimidines, triazines, azepines and their benzo-fused representatives, and purines).

Conversion of compounds of the general formula:

HNR_{E}¹—(CR_{E}³R_{E}⁴)ₖ₁—(L_{E})ₖ₂—(CR_{E}⁵R_{E}⁶)ₖ₅-(Q_{E})ₖ₄—(CR_{E}⁷R_{E}⁸)ₖ₅—(T_{E})ₖ₅—(CR_{E}⁹R_{E}¹⁰)ₖ₇—(U_{E})ₕ-D_{E} (VIII)

NC—(CR_{E}³R_{E}⁴)ₖ₁-(L_{E})ₖ₂—(CE_{E}⁵R_{E}⁶)ₖ₃-(Q_{E})ₖ₄—(CR_{E}⁷R_{E}⁸)ₖ₅—(T_{E})ₖ₆—(CR_{E}⁹R_{E}¹⁰)ₖ₇—(U_{E})ₕ-D_{E} (IX)

into compounds of the general formula:

A—(CR_{E}³R_{E}⁴)ₖ₁-(L_{E})(CR_{E}⁵R_{E}⁶)ₖ₅-(Q_{E})ₖ₄—(CR_{E}⁷R_{E}⁸)ₖ₅—(T_{E})ₖ₆—(CR_{E}⁹R_{E}¹⁰)ₖ₇—(U_{E})ₕ-D_{E} (X)

A-(CR_{E}³Rₑ⁴)ₖ₁-(L_{E})ₖ₂-(CR_{E}⁵R_{E}⁶)ₖ₂-(Q_{E})ₖ₄—(CR_{E}⁷R_{E}⁸)ₖ₅—(T_{E})ₖ₆—(CR_{E}⁹R_{E}¹⁰)ₖ₇-(U_{E})ₕ-D_{E} (XI)

can take place by methods known to the skilled worker, as described, for example, in WO 97/08145. The group D_{E} in the formulae VIII-XI is a radical having the meaning H or NSG₂. These building blocks can then be reacted either directly or after elimination of the appropriate protective group SG₂ to give compounds of the general formula I (Scheme 2).

Schemes 3-7 describe a number of methods for introducing A by way of example, the reaction conditions used in each case being those known and suitable for the particular reactions. It is moreover possible to make use of variants which are known per se but not mentioned here.

Ureas and thioureas (AE-1 to AE-3) can be prepared by conventional methods of organic chemistry, for example by reacting an isocyanate or isothiocyanate with an mine, where appropriate in an inert solvent with heating (Houben-Weyl, volume VIII, 157 et seq.) (Scheme 3)

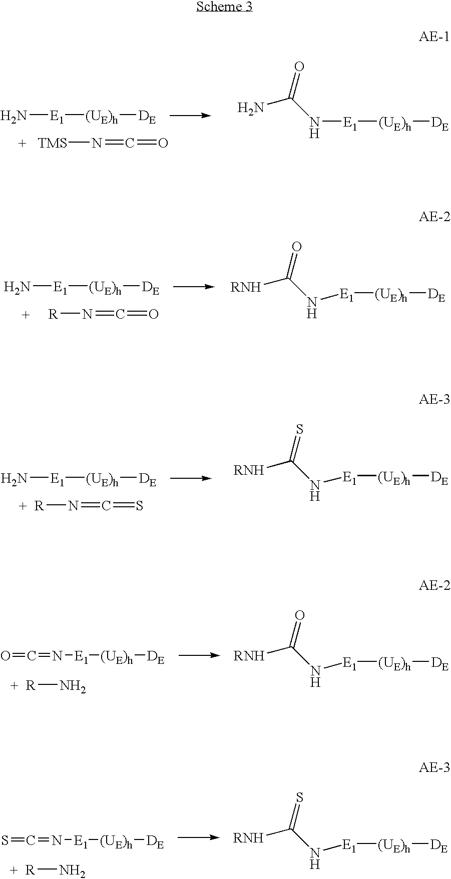

Scheme 4 shows by way of example the preparation of compounds of type AE-4 as described, for example, by Blakemoore et al. in Eur. J. Med. Chem. 1987 (22) 2, 91-100, or by Misra et al. in Bioorg. Med. Chem. Let. 19944(18), 2165-2170.

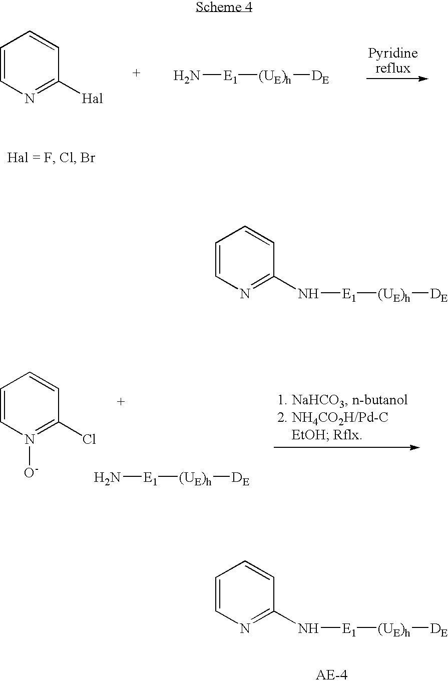

Unsubstituted or cycl. guanidine derivatives of the general formula AE-5 and AE-6 can be prepared using reagents which can be purchased or obtained simply, as described, for example, in Synlett 1990, 745, J. Org. Chem. 1992, 57, 2497, Bioorg. Med. Chem. 1996, 6, 1185-1208; Bioorg. Med. Chem. 1998, 1185, or Synth. Comm. 1998, 28, 741-746.

Preparation of compounds of the general formula AE-7 can take place in analogy to U.S. Pat. No. 3,202,660, and of compounds of the formula AE-9, AE-10, AE-11 and AE-12 in analogy to WO 97/08145. Compounds of the formula AE-8 can be prepared as shown in. Scheme 5, for example by the method described by Perkins et al., Tetrahedron Lett. 1999, 40, 1103-1106. Scheme 5 summarizes the synthesis of said compounds.

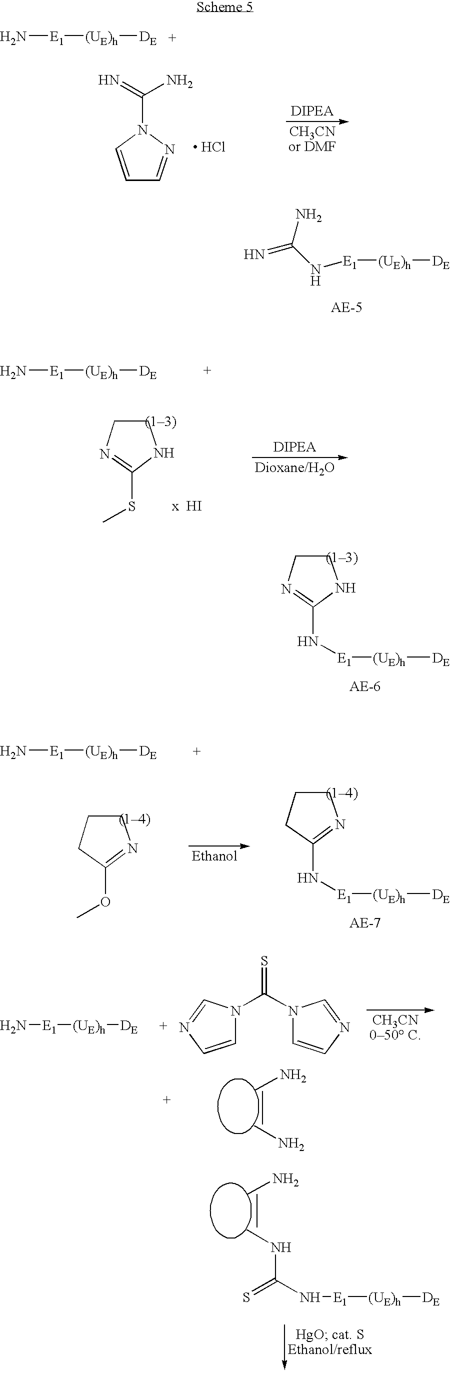

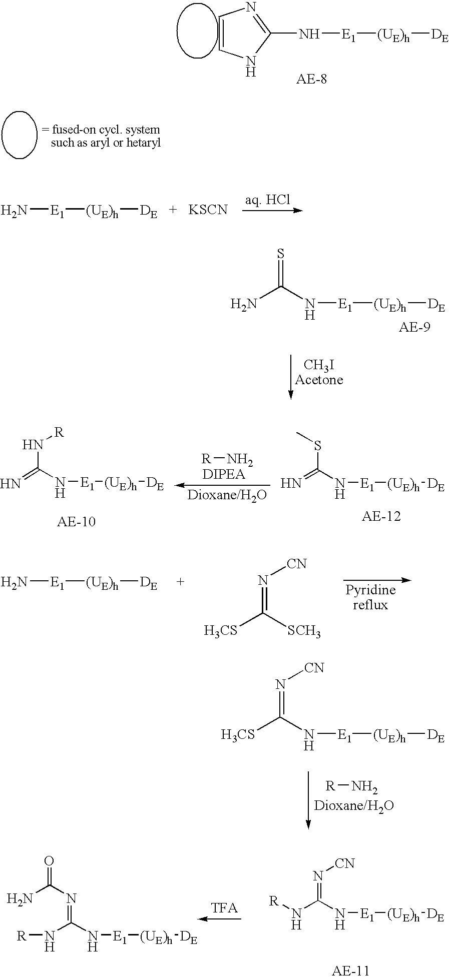

Compounds of the general formula AE-13 can be prepared in analogy to Froeyen et al., Phosphorus Sulfur Silicon Relat. Elem. 1991, 63, 283-293, and AE-14 in analogy to Yoneda et al., Heterocycles 1998, 15 N′-1, Spec. Issue, 341-344 (Scheme 6). Corresponding compounds can also be prepared in analogy to WO 97/36859:

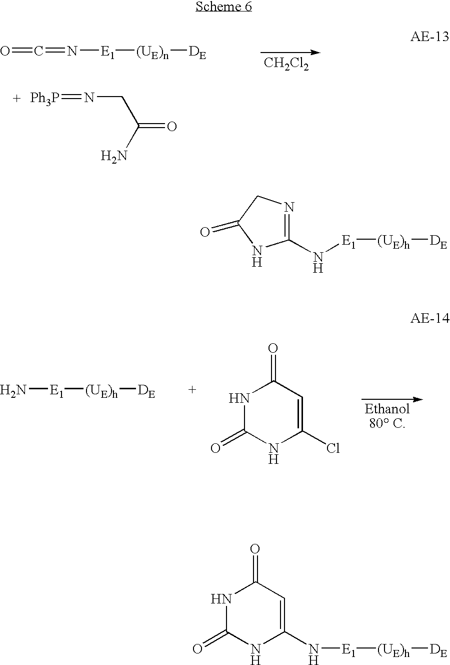

Compounds of the general formula AE-15 can be prepared as in Synthesis 1981, 963-965 or Synth. Comm. 1997, 27 (15), 2701-2707, and AE-16 in analogy to J. Org. Chem. 1991, 56(6), 2261-2262 (Scheme 7).

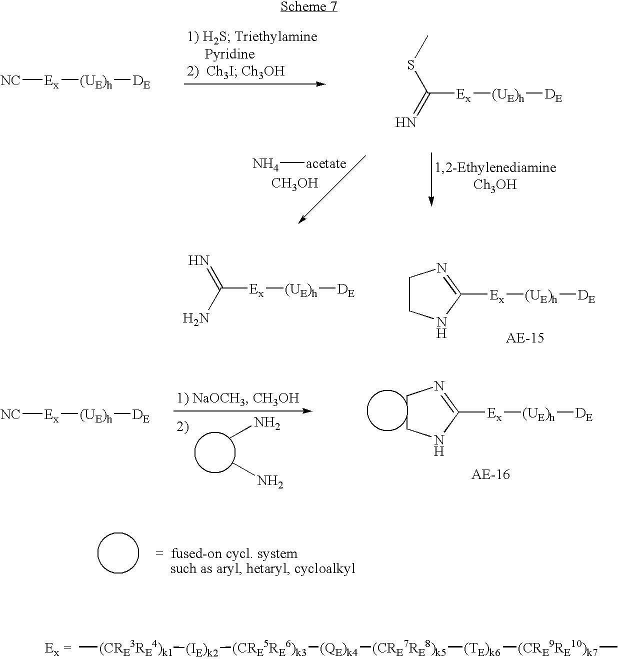

The invention further relates to the use of the structural element of the formula I_{GL}

-G-L I_{GL}

for preparing compounds which bind to integrin receptors.

The invention further relates to drugs containing the structural element of the formula I_{GL}.

The invention further relates to pharmaceutical preparations containing at least one compound of the formula I in addition to conventional pharmaceutical excipients.

The compounds according to the invention can be administered orally or parenterally (subcutaneously, intravenously, intramuscularly, intraperitoneally) in a conventional way. Administration can also take place with vapors or sprays through the nasopharyngeal space. The compounds according to the invention can also be introduced by direct contact with the affected tissue.

The dosage depends on the age, condition and weight of the patient and on the mode of administration. The daily dose of active ingredient is usually between about 0.5 and 50 mg/kg of body weight on oral administration and between about 0.1 and 10 mg/kg of body weight on parenteral administration.

The novel compounds can be used in conventional solid or liquid pharmaceutical forms, for example as uncoated or (film-) coated tablets, capsules, powders, grandules, suppositories, solutions, ointments, creams or sprays. These are produced in a conventional way. The active ingredients can for this purpose be processed with conventional pharmaceutical excipients such as tablet binders, bulking agents, preservatives, tablet disintegrants, flow regulators, plasticizers, wetting agents, dispersants, emulsifiers, solvents, release-slowing agents, antioxidants and/or propellant gases (cf. H. Sucker et al.: Pharmazeutische Technologie, Thieme-Verlag, Stuttgart, 1991). The administration forms obtained in this way normally contain the active ingredient in an amount of from 0.1 to 90% by weight.

The invention further relates to the compounds of the formula I for use as drugs, and to the use of the compounds of the formula I for producing drugs for treating diseases. The compounds of the formula I can be used for treating human and animal diseases. The compounds of the formula I bind to Integrin receptors. They are therefore suitable preferably as integrin receptor ligands and for producing drugs for treating diseases In which an integrin receptor is involved, in particular for treating diseases associated with dysregulation, that is to say an increase or decrease, of the interaction between integrins and their natural ligands.

Integrin receptor ligands mean agonists and antagonists.

An increased or decreased interaction means both an increased or decreased expression of the natural ligand and/or of the integrin receptor and thus an increased or decreased amount of natural ligand and/or integrin receptor or an increased or decreased affinity of the natural ligand for the integrin receptor.

There is dysregulation of the interaction between integrins and their natural ligands compared with the normal state, that is to say an increase or decrease, when this dysregulation does not correspond to the physiological state. An increased or decreased interaction may lead to pathophysiological situations.

The level of dysregulation leading to a pathophysiological situation depends on the individual organism and on the site and nature of the disorder.

Preferred integrin receptors for which the compounds of the formula I according to the invention can be used are the α₅β₁, α_{V}β₅, αᵥβ₅ and αᵥβ₃ integrin receptors.

It is particularly preferred for the compounds of formula I to bind to the αᵥβ₃ integrin receptor and they can thus be used particularly preferably as ligands of the αᵥβ₃ integrin receptor and for treating diseases in which the interaction between αᵥβ₃ integrin receptor and its natural ligand is increased or decreased.

The compounds of the formula I are preferably used for treating the following diseases or for producing drugs for treating the following diseases:

cardiovascular disorders such as atherosclerosis, restenosis after vessel injury or stent implantation, and angioplasty (neointima formation, smooth muscle cell migration and proliferation), acute kidney failure,

angiogenesis-associated microangiopathies such as, for example, diabetic angiopathies or retinopathy or rheumatoid arthritis,

vascular occlusion mediated by blood platelets, arterial thrombosis,

stroke, reperfusion damage after myocardial infarction or stroke,

cancers such as, for example, in tumor metastasis or tumor growth (tumor-induced angiogenesis),

osteoporosis (bone resorption after chemotaxis and adhesion of osteoclasts to bone matrix),

high blood pressure, psoriasis, hyperparathyroidism, Paget's disease, malignant hypercalcemia, metastatic osteolytic lesions, inflammation, wound healing, heart failure, congestive heart failure CHF, and for

antiviral, antimycotic, antiparasitic or antibacterial therapy and prophylaxis (adhesion and internalization).

The compounds of the formula I can advantageously be administered in combination with at least one other compound in order to achieve an improved curative effect in a number of indications. These other compounds may have the same or a different mechanism of action as the compounds of the formula I.

The pharmaceutical preparations may therefore contain, besides the compounds of the formula I and the conventional pharmaceutical excipients, at least one other compound, depending on the indication in each case selected from one of the following 10 groups.

Group 1:

inhibitors of blood platelet adhesion, activation or aggregation, such as, for example, acetylsalicylic acid, lysine acetylsalicylate, piracetam, dipyridamole, abcixlmab, thromboxane antagonists, fibrinogen antagonists such as, for example, tirofiban, or inhibitors of ADP-induced aggregation such as, for example, ticlopidine or clopidogrel, anticoagulants which impede thrombin activity or formation, such as, for example, inhibitors of IIa, Xa, XIa, IXa or VIIa,

antagonists of blood platelet-activating compounds and

selectin antagonists

for the treatment of vascular occlusion mediated by blood platelets, or thrombosis, or

Group 2:

inhibitors of blood platelet activation or aggregation such as, for example, GPIIb/IIIa antagonists, thrombin inhibitors or factor Xa inhibitors or ADP receptor antagonists,

serine protease inhibitors,

fibrinogen-reducing compounds,

selectin antagonists,

antagonists of ICAM-1 or VCAM-1

inhibitors of leukocyte adhesion

inhibitors of vessel wall transmigration,

fibrinolysis-modulating compounds such as, for example, streptokinase, tPA,

plasminogen activation stimulants, TAFI inhibitors, XIa inhibitors or PAI-1 antagonists,

inhibitors of complement factors,

endothelin receptor antagonists,

tyrosine kinase inhibitors,

antioxidants and

interleukin 8 antagonists

for the treatment of myocardial infarct or stroke, or

Group 3:

endothelin antagonists,

ACE inhibitors,

angiotensin receptor antagonists,

endopeptidase inhibitors,

beta blockers,

calcium channel blockers,

phosphodiesterase inhibitors and

caspase inhibitors

for the treatment of congestive heart failure, or

Group 4:

thrombin inhibitors,

inhibitors of factor Xa,

inhibitors of the coagulation pathway leading to thrombin formation, such as, for example, heparin or low molecular weight heparins,

inhibitors of blood platelet adhesion, activation or aggregation, such as, for example,

GPIIb-IIIa antagonists or antagonists of blood platelet adhesion and activation mediated by vWF or GPIb,

endothelin receptor antagonists,

nitric oxide synthase inhibitors,

CD44 antagonists,

selectin antagonists,

MCP-1 antagonists,

inhibitors of signal transduction in proliferating cells,

antagonists of the cellular response mediated by EGF, PDGF, VEGF or bFGF and antioxidants

for the treatment of restenosis after vessel injury or stent implantation, or

Group 5:

antagonists of the cellular response mediated by EGF, PDGF, VEGF or bFGF,

heparin or low molecular weight heparins or other GAGs,

inhibitors of MMPs,

selectin antagonists,

endothelin antagonists,

ACE inhibitors,

angiotensin receptor antagonists and

glycosylation inhibitors or AGE formation inhibitors or AGE breakers and antagonists of their receptors such as, for example, RAGE,

for the treatment of diabetic angiopathies or

Group 6:

lipid-lowering compounds,

selectin antagonists,

antagonists of ICAM-1 or VCAM-1

heparin or low molecular weight heparins or other GAGs,

inhibitors of MMPs,

endothelin antagonists,

apolipoprotein A1 antagonists,

cholesterol antagonists,

HMG-CoA reductase inhibitors,

ACAT inhibitors,

ACE Inhibitors,

angiotensin receptor antagonists,

tyrosine kinase inhibitors,

protein kinase C inhibitors,

calcium channel blockers,

LDL receptor function stimulants,

antioxidants

LCAT mimetics and

free radical scavengers

for the treatment of atherosclerosis or

Group 7:

cytostatic or antineoplastic compounds,

compounds which inhibit proliferation, such as, for example, kinase inhibitors and heparin or low molecular weight heparins or other GAGs

for the treatment of cancer, preferably for inhibiting tumor growth or metastasis, or

Group 8:

compounds for antiresorptive therapy,

compounds for hormone replacement therapy such as, for example, estrogen or progesterone antagonists,

recombinant human growth hormone,

bisphosphonates such as, for example, alendronate

compounds for calcitonin therapy,

calcitonin stimulants,

calcium channel blockers,

bone formation stimulants such as, for example, growth factor agonists,

interleukin-6 antagonists and

Src tyrosine kinase inhibitors

for the treatment of osteoporosis or

Group 9:

TNF antagonists,

antagonists of VLA-4 or VCAM-1,

antagonists of LFA-1, Mac-1 or ICAMS,

complement inhibitors,

immunosuppressants,

interleukin-1, or antagonists and

dihydrofolate reductase inhibitors

for the treatment of rheumatoid arthritis or

Group 10:

collagenase,

PDGF antagonists and

MMPs

for improved wound healing.

A pharmaceutical preparation containing at least one compound of the formula 1, where appropriate pharmaceutical excipients and at least one other compound, depending on the indication in each case selected from one of the above groups, means a combined administration of at least one of the compounds of the formula I with at least one other compound in each case selected from one of the groups described above and, where appropriate, pharmaceutical excipients.

Combined administration can be effected by a mixture of substances containing at least one compound of the formula I, where appropriate pharmaceutical excipients and at least one other compound, depending on the indication in each case chosen from one of the above groups, but also spatially and/or temporally separate.

For spatially and/or temporally separate administration, the components of the pharmaceutical preparation, the compounds of the formula I and the compounds selected from one of the aforementioned groups, are administered spatially and/or temporally separately.

For the treatment of restenosis after vessel injury or stenting, the administrations of the compounds of the formula I alone or in combination with at least one compound selected from Group 4 can take place locally at the affected sites. It may also be advantageous to coat the stents with these compounds.

For the treatment of osteoporosis, it may be advantageous to carry out administration of compounds of the formula I in combination with an antiresorptive or hormone replacement therapy.

The invention accordingly relates to the use of the aforementioned pharmaceutical preparations for producing drugs for the treatment of diseases.

In a preferred embodiment, the invention relates to the use of the aforementioned combined pharmaceutical preparations for producing drugs for the treatment of

vascular occlusion mediated by blood platelets, or thrombosis

on use of compounds of group 1,

myocardial infarct or stroke

on use of compounds of group 2,

congestive heart failure

on use of compounds of group 3,

restenosis after vessel injury or stent implantation

on use of compounds of group 4,

diabetic angiopathies

on use of compounds of group 5,

atherosclerosis

on use of compounds of group 6,

cancer

on use of compounds of group 7,

osteoporosis

on use of compounds of group 8,

rheumatoid arthritis

on use of compounds of group 9,

wound healing

on use of compounds of group 10.

The following examples illustrate the invention, the selection of these examples being non-limiting.

### I. SYNTHESIS EXAMPLES

I.A Precursors

### Example 1

N-(Piperidin-4-ylmethyl)-1H-benzimidazol-2-amine(trifluoroacetate)(1)

a.) A solution of 1-(tert-butyloxycarbonyl)-4-(aminomethyl)piperidine (5.39 g; 25 mmol) in 25 ml of —CH₃CN was added dropwise to 6.75 g of thiocarbonyldiimidazole and 0.5 g of imidazole in 100 ml of CH₃CN at 0° C. and then stirred at RT for 3 h. 1,2-Phenylene-diamine (5.5 g; 50.86 mmol) was then added, and the mixture was heated at 60° C. for about 1 h. The solid which resulted on cooling was filtered off with suction and dried. 6.79 g; ESI-MS [M+H⁺-⁺Bu]=309.15;

b.) 1-(tert-Butyloxycarbonyl)-4-({[(2-aminoanilino)-thiocarbonyl]amino}methyl)piperidine (1a) (5 g; 13.72 mmol), 5.94 g of HgO (yellow) and 0.6 g of sulfur in 150 ml of ethanol were heated to reflux for 1 h. The mixture was filtered 2× through Celite and evaporated, and the resulting crude product was purified by chromatography on silica gel (CH₂Cl₂/CH₃OH 5→25%).

2.65 g; ESI-MS [M+H⁺]=331.25

1H-NMR (360 MHz, DMSO) δ ppm: 7.15 and 6.9 (each m, 2H), 3.95 (d, 2H), 3.2 (m 2H), 2.7 (br m; 2H), 1.8 (m, 1H), 1.7 (m, 2H), 1.35 (s, 9H), 1.05 (m, 2H).

c.) 1-(tert-Butyloxycarbonyl)-4-[(1H)-benzimidazol-2-ylamino)methyl]piperidine (1b) (2.65 g; 8.02 mmol) was treated with 10 ml of TFA under standard conditions. Concentration and stirring of the crude product with n-pentane afforded 2.3 g; ESI-MS

[M+H⁺]=231.15.

1H-NMR (360 MHz, DMSO) δ ppm: 13.25 (s, 1H), 9.35 (m, 1H), 8.8 and 8.5 (each br s, 1H), 7.4 and 7.20 (each m, 2H), 3.3 (m, 4H), 2.85 (m, 2H), 1.9 (m, 3H), 1.35 (m, 2H).

### Example 2

1-(tert-Butyloxycarbonyl)-4-[(2-pyridinylamino)methyl]piperidine (2)

1-(tert-Butyloxycarbonyl)-4-(aminomethyl)piperidine (3 g; 14 mmol) and 10 ml of 2-fluoropyridine were heated to reflux for 4 h. Concentration and stirring of the crude product in n-pentane afforded 3 g of a white solid, m.p.: 126-130° C.; ESI-MS [M+H⁺]=292.15.

The amine required for further reaction was obtained by elimination of the Boc group with HCl in dioxane (under standard conditions); the isolated HCl salt then employed directly.

### Example 3

N-[4-(Aminomethyl)-1,3-thiazol-2-yl]-N′-benzylurea (hydrochloride) (3)

a.) A solution of 123 g of pyridinium bromide perbromide in 600 ml of THF was slowly added dropwise to 2-(2-oxopropyl)-1H-isoindole-1,3(2H)-dione (70 g; in 0.345 mol) in 600 ml of THF, and the mixture was then stirred for about 3 h. For work up, the precipitated solid was filtered off, and the mother liquor was concentrated, taken up in ethyl acetate and thoroughly washed with aqueous bisulfite solution. Drying and concentration afforded 150 g of a yellow oil, which was stirred with methyl ten-butyl ether.

63.4 g; m.p.: 142-143° C.; ESI-MS [M+H⁺]=283.95

b.) 2-(3-Bromo-2-oxopropyl)-1H-isoindole-1,3 (2H)-dione 3a (6 g; 21.27 mmol) and thiourea (2 g; 26.27 mmol) were stirred in 70 ml of THF at RT for about 2 h. The resulting precipitate was filtered off with suction and dried. 5 g; ESI-MS [M+H⁺]=260.05

c.) 2-[(2-Amino-1,3-thiazol-4-yl)methyl]-1H-isoindole-1,3(2H)-dione hydrobromide 3b (4.5 g; 13.23 mmol), benzyl isocyanate (1.8 g; 13.52 mmol) and 1.7 g of DIPEA were heated to reflux in 50 ml of toluene. After the reaction was complete, the mixture was evaporated and the residue was taken up In CH₂Cl₂ and washed with 1 N HCl, saturated NaHCO₃ and NaCl solutions. Drying and concentration afforded 4.7 g of an orange solid, which was recrystallized from CH₃OH.

3.0 g; ESI-MS [M+H⁺]=393.05

1H-NMR (360 MHz, DMSO) δ ppm: 10.65 (s, 1H), 7.9 (m, 4H), 7.25 (m, 5H), 6.85 (s, 1H), 4.7 (s, 2H), 4.35 (d, 2H),

d). N-Benzyl-N-{4-[(1,3-dioxo-1,3-dihydro-2H-isoindol-2-yl)methyl]-1,3-thiazol-2-yl}urea 3c (3 g; 7.64 mmol) was suspended in 50 ml of CH₃OH and, after addition of 2 g of hydrazine hydrate, stirred at RT for 2 h. The resulting solid was filtered off, and the resulting mother liquor was evaporated and stirred with 0.5N HCl. Renewed filtration and evaporation of the mother liquor led to concentration of the desired product, and this purification step was therefore repeated 3×.

0.78 g; ESI-MS [M+H⁺]=263.05

### Example 4

2-(Piperidin-4-ylamino)pyridine (4)

a.) Ethyl 4-amino-1-piperidinecarboxylate (6 g, 34.8 mmol) and 25 g of 2-fluoropyridine were heated to reflux for 48 h. The solid which formed after cooling was filtered off with suction, stirred with n-pentane and dried; 6.26 g of yellow powder; ESI-MS [M+H⁺]=250.15.

b.) 6 g of ethyl 4-(pyridin-2-ylamino)piperidin-1-carboxylate 4a were heated in 30 ml of 47% HBr to reflux for 6 h. Evaporation of the mixture, stirring of the resulting crude product with ethyl acetate/CH₃OH (9:1) and renewed drying afforded 7.1 g of white solid; ESI-MS [M+H⁺]=178.15.

### Example 5

2-Piperidinyl-4-yl-1H-benzimidazole (5)

Preparation analogous to J. Heterocycl. Chem. 26, 1989, 541-543.

### Example 6

N-Piperidin-4-yl-1H-benzimidazol-2-amine (trifluoroacetate) (6)

a.) Reaction In analogy to I starting from Boc-4-aminopiperidine hydrochloride (14 g; 59.14 mmol) afforded 19.1 g of brown solid as crude product which was purified by chromatography on silica gel (CH₂Cl₂/CH₃OH/MTB 1:1:1) and, after stirring with n-pentane, 11.2 g of white solid were obtained; ESI-MS [M+H⁺]=317.15.

¹H-NMR (360 MHz, DMSO) δ ppm: 10.7 (broad, 11H), 7.15 and 6.85 (each m, 2H), 6.55 (d, 1H), 3.90 (m, 2H), 3.80 (m, 1H), 2.90 (m, 2H), 1.95 (m, 2H), 1.45 (s, 9H), 1.30 (m, 2H).

b.) Boc cleavage with TFA and precipitation of the trifluoroacetate from MTB/CH₃OH 10:1 afforded 13 g of solid; ESI-MS [M+H⁺]=217.5.

### Example 7

N-(1H-Imidazol-2-yl)pyrrolidine-3-carboxamide (trifluoroacetate) (7)

Coupling of Boc-pyrrolidine-3-carboxylic acid (0.5 g; 2.32 mmol) with 2-aminoimidazole (0.19 g; 2.32 mmol) in 15 ml of DMF using TBTU as coupling reagent and N-methyl-morpholine as base afforded, after precipitation of the crude product from CH₂/Cl₂/diethyl ether, 0.2 g of white solid; ESI-MS [M+H⁺]=281.25. Cleavage of the Boc group with TFA under standard conditions and precipitation of the product from ethyl acetate/diethyl ether afforded 0.7 g.

### Example 8

2-(N-Carbethoxythiocarbamoyl)-1-(N-piperidino)-1-propene (8)

40 ml of piperidine were added dropwise to 11.85 g (0.2 mol) of propionaldehyde and 10 g of K2CO₃ at 0° C. The mixture was subsequently stirred at this temperature for 2 h, nd then the insolubles were filtered off and the filtrate was fractionated in vacuo.

Ethoxycarbonyl isothiocyanate (63.7 g; 0.48 mol) was added dropwise to the solution of freshly distilled 1-(N-piperidino)propene (9.58 g; 0.077 mol) in 40 ml of dry diethyl ether while cooling; an orange-red precipitate formed during the addition. The reaction mixture was stirred further at 0-5° C. for about 4 h, and the precipitate was filtered, washed and dried. The residue remaining after evaporation of the mother liquor was again treated with diethyl ether and filtered.

Yield: 6.81 g

¹H-NMR (400 MHz, CDCl₃): δ (ppm) 7.85 (s, 1H; CH═C), 7.70 (br, 1H, NH), 4.15 (q, 2H, CH₂), 3.6 (m, 4H, piperidine); 2.2 (s, 3H, CH₃), 1.7 (m, 6H, piperidine), 1.3 (t, 3H, CH₃).

### Example 9

### 2-(N-Carbethoxythiocarbamoyl)-1-(N-piperidino)-2-phenylethene (9)

17.04 g=19.8 ml (0.2 mol) of piperidine were slowly added to 15.8 ml (0.1 mol) of a 50% strength solution of phenylacetaldehyde in diethyl phthalate and 5 g of K₂CO₃ at 0° C. The mixture was then stirred at 0° C. to 5° C. for 1.5 h. The insolubles were subsequently filtered off with suction, and the mother liquor was distilled under oil pump vacuum to a bath temperature of 80° C. The yellow oil obtained as residue (27.15 g, contains about 50% diethyl phthalate) was introduced into 40 ml of abs. diethyl ether under nitrogen at 0° C. 8.1 ml=9.02 g (80 mmol) of ethoxycarbonyl isothiocyanate were slowly injected at 0° C. The mixture was then stirred at 0° C. to 5° C. for 4 h, an orange solid precipitating after about 30 min. The solid was filtered off with suction under N₂, washed with diethyl ether and dried under a stream of N₂.

Yield 21.5 g of yellow solid.

1H-NMR (400 MHz, CDCl₃): δ (ppm) 8.45 (s, 1H; CH═C), 7.65 (br, 1H, NH), 7.35 (m, 5H, phenyl), 4.1 (q, 2H, CH₂), 3.1 (m, 4H, piperidine), 1.5 (m, 6H, piperidine), 1.15 (t, 3H, CH3).

### Example 10

2-(N-Carbethoxythiocarbamoyl)-1-(N-piperidino)-1-pentene (10)

The enamine obtained from 17.23 g (0.2 mol) of valeraldehyde, 10 g of K₂CO₃ and 39.6 ml of piperidine was reacted with 8.84 ml (74.9 mmol) of ethoxylcarbonyl isothiocyanate.

Yield: 15.15 g of dark yellow solid.

1H-NMR (270 MHz, CDCl₃): δ (ppm) 7.77 (br, 1H, NH), 7.52 (s, 1H; CH═C), 4.15 (q, 2H, CH₂), 3.5 (m, 4H, piperidine), 2.7 (t, 2H, CH₂), 1.7 (m, 6H, piperidine), 1.55 (m, 2H, CH₂), 1.3 (t, 3H, CH₃), 0.95 (t, 3H, CH₃).

### Example 11

2-(N-Carbethoxythiocarbamoyl)-2-(tetrahydro-2H-pyran-4-yl)-1 (N-piperidino)-ethene (11)

The enamine obtained from 25.84 g (0.32 mol) of 4-formyltetrahydropyran, 10 g of K₂CO₃ and 39.6 ml of piperidine was reacted with 10.1 ml (85.6 mmol) of ethoxycarbonyl isothiocyanate.

Yield: 27 g of yellow solid.

1H-NMR (270 MHz, CDCl₃): δ (ppm) 8.2 (broad, 1H, NH), 6.84 (s, 1H; CH═C), 4.15 (q, 2H, CH₂), 3.95 and 3.5 (each m, 2H, THP-OCH₂), 3.2 (m, 4H, piperidine-NHC₂), 2.65 (m, 1H, THP-CH), 1.65-1.95 (m, 10H, piperidine-CH₂ and THP-CH₂), 1.3 (t, 3H, CH₃).

### Example 12

2-(N-Carbethoxythiocarbamoyl)-1-(N-morpholin-4-yl)-4-phenylbut-1-ene (12)

The enamine obtained from 48 g (0.2 mol) of phenylbutyraldehyde, 30 g of K₂CO₃ and 95 g of morpholine was reacted with 35 g of ethoxycarbonylisothiocyanate.

Yield: 57.7 g of yellow solid.

1H-NMR (360 MHz, CDCl₃): δ (ppm) 7.95 (broad, 1H, NH), 7.5 (s, 1H; CH═C), 7.45-7.15 (m, 5H), 4.15 (q, 2H, CH₂), 3.75 and 3.45 (each m, 4H, morpholine), 3.05 and 2.85 (each m, 2H), 1.28 (t, 3H, CH₃).

### Example 13

2-(N-Carbethoxythiocarbamoyl)-1-(N-morpholin-4-yl)-3-phenylpropen-1-ene (13)

30.1 g of the enamine obtained from reaction of phenylpropionaldehyde with morpholine was reacted with 19.7 g of ethoxycarbonyl isothiocyanate.

Yield: 22 g of yellow solid.

I.B. Compounds of the General Formula I

### Example 1

2-[(1-{1-[2-Carboxy-3-(2-naphthyl)propyl]-5-methyl-2-oxo-1,2-dihydropyrimidin-4-yl)-piperidin-4-yl)amino]-1H-benzimidazol-1-ium trifluoroacetate

a.) 1.04 ml (5.6 mmol) DIPEA and 1.06 g (2.35 mmol) of N-[(9H-fluoren-9-yl-methoxy)carbonyl]-2-(2-naphthylmethyl)-β-alanine (Rare Chemicals), dissolved in a little DMF, were successively added to a suspension of 1.95 g of 2-Cl-trityl-resin (1.4 mmol Cl/g resin) in CH₂Cl₂. After incubation at room temperature for 16 h, 1.7 ml of DIPEA and 9.75 ml of MeOH were added, and the mixture was shaken at room temperature for 1 h. It was then filtered with suction, and washed with DMF, CH₂Cl₂, CH₃OH and CH₂Cl₂.

The N-[(9H-fluoren-9-ylmethoxy)carbonyl]-2-(2-naphthylmethyl)-β-alanine-2-Cl-trityl-resin obtained in this way was treated twice for 20 min each with piperidine in DMF (50%) and then washed with DMF, CH₂Cl₂, MeOH and CH₂Cl₂. Drying in vacuo resulted in 2.42 g of the deprotected resin (substitution 1.01 mmol of amino acid/g of resin).

b.) 1 g (1 mmol) of the resin obtained in this way was suspended in DMF and, after addition of 768 mg (3 mmol) of 2-(N-carbethoxythiocarbamoyl)-1-(N-piperidino)-3-propene 8, incubated at RT overnight. It was then washed with DMF and CH₂Cl₂.

c.) 136 m (0.12 mmol) of the resin obtained in this way was suspended in 7 ml of NMP, and 210 μl (1.2 mmol) of DIPEA and 0.12 ml of a 5M BrCN solution in CH₃CN were added. Incubation at room temperature for 16 h was followed by filtration with suction and washing with NMP and CH₂Cl₂. The resin was then suspended in 7 ml of NMP and, after addition of 250 μl (1.44 mmol) of DIPEA, 106.65 mg (0.24 mmol) of 6 were added. Incubation overnight was followed by filtration with suction and washing with DMF, H₂O, DMF, CH₂Cl₂, MeOH and CH₂Cl₂. Cleavage of the product from the resin was carried out with 3 ml of trifluoroethanovglacial acetic acid/CH₂Cl₂ (1 h, RT). Filtration was followed by evaporation, taken up in 2 ml of glacial acetic acid and lyophilization. The crude product was purified by preparative RP-HPLC (MeOH/H₂O/0.1% trifluoroacetic acid).

Yield: 27.4 mg

ESI-MS [M+H)⁺: 537 (MG: 536.6)

1H-NMR (360 MHz, DMSO): δ ppm: 9.10 (d, 1H), 8.90-8.80 (m, 3H), 8.70 (s, 1H), 7.50-7.20 (m, 7H), 4.10 (m, 2H), 3.15-2.95 (m 4H), 2.05 (m, 2H), 1.95 (s, 3H), 1.70-1.55 (m, 2H).

The following were prepared analogously:

### Example II

2-([(1{1-[(2S)-2-Carboxy-3-phenylpropyl]-5-methyl-2-oxo-1,2-dihydropyrimidin-4yl]-piperidin-4-yl)methyl}amino]pyridinium acetate

ESI-MS [M+H]⁺: 463 (MW: 461.6)

### Example III

2-[((1-[1-(2-Carboxy-3-phenylpropyl)-5-methyl-2-oxo-1,2-dihydropyrimidin-4-yl]pyrrolidin-3-yl}carbonyl)amino]-1H-imidazol-1-ium acetate

ESI-MS [M+H]⁺: 451 (MW: 450.5)

### Example IV

2-[(1-(1-[(2S)-2-Carboxy-3-phenylpropyl]-5methyl-2-oxo-1,2-dihydropyrimidin-4-yl)piperidin-4-yl)pyrrolidin-4-yl)amino]-1H-benzimidazol-1-ium acetate

ESI-MS [M+H]⁺: 487 (MW: 486.6)

### Example V

2-(1-(1-[(2S)-2-Carboxy-3-phenylpropyl]-5-methyl-2-oxo-1,2-dihydropyrimidin-4-yl}-piperidin-4-yl)-1H-benzimidazol-1-ium Acetate

ESI-MS [M+H]⁺: 472 (MW: 471.6)

### Example VI

3-(4-{[(2-{[(Benzylamino)carbonyl]amino}-1,3-thiazol-4-yl)methyl]amino}-5-methyl-2-oxopyrimidin-1(2H)-yl)-2-(2-naphthylmethyl)propanoic acid

ESI-MS [M+H]⁺: 583 (MW: 582.17)

### Example VII

2-Benzyl-3-(4-[[(2-[[(benzylamino)carbonyl]amino]-1,3-thiazolyl)methyl]amino]-5-methyl-2-oxopyrimidin-1 (2H)-yl)propanoic acid

ESI-MS [M+H]⁺: 533 (MW: 532.16)

### Example VIII

2-[[(1-{1-[2-Carboxy-3-(₂-phenylpropyl)-5-methyl-2-oxo-1,2-dihydropyrimidinyl-4-yl]piperidin-4-yl)methyl]amino)-1H-benzimidazol-1-ium triluoroacetate

ESI-MS [M+H]⁺: 501 (MW: 500.6)

### Example IX

2-[[(1-(1-[2-Carboxy-3-(2-naphthyl)propyl]-5-methyl-2-oxo-1,2-dihydropyrimidin-4-yl]-piperidin-4-yl)methyl]amino]-1H-benzimidazol-1-ium trifluoroacetate

E-SI-MS [M+H]⁺551 (MW: 550.7)

### Example X

2-[[(1-[1-[2-Carboxy-3-(2-naphthyl)propyl]-5-methyl-2-oxo-1,2-dihydropyrimidin-4-yl]-piperidin-4-yl)methyl]amino}pyridinium trifluoroacetate

ESI-MS [M+H]⁺: 512 (MW: 511.6)

### Example XI

2-[(1-[2-Carboxy-3-(2-naphthyl)propyl]-5-methyl-2-oxo-1,2-dihydropyrimidin-4-yl]-piperidin-4-yl)amino]pyridinium trifluoroacetate

ESI-MS [M+H]⁺: 498 (MW: 497.6)

### Example XII

2-{[(1-{1-[2-Carboxy-3-(2-naphthyl)propyl]-5-methyl-2-oxo-1,2-dihydropyrimidin-4-yl}-pyrrolidin-3-yl)carbonyl]amino}-1H-imidazol-3-ium Trifluoroacetate

ESI-MS [M+Hr^{+]}: 501 (MW: 500.6)

### Example XIII

2-(1-(1-[2-Carboxy-3-(2-naphthyl)propyl]-5-methyl-2-oxo-1,2-dihydropyrimidin-4-yl)-piperidin-4-yl)-1H-benzimidazol-1-ium trifluoroacetate

ESI-MS [M+H]⁺: 522 (MW: 521.6)

### Example XIV

2-Benzyl-3-(4-{[(2-{[(benzylamino)carbonyl]amino}-1,3-thiazol-4-yl)methyl]amino}-5-methyl-2-oxopyrimidin-1(2H)-yl)propanoic Acid

ESI-MS [M+H]⁺: 533 (MW: 532.6)

### Example XV

2-{[(1-[2-Carboxy-3-(2-naphthyl)propyl]-2-oxo-5-phenyl-1,2-dihydropyrimidin-4-yl}-pyrrolidin-3-yl)carbonylamino]-1H-imidazol-3-ium trifluoroacetate

ESI-MS [M+H]⁺: 563 (MW: 562.6)

### Example XVI

2-({1-[1-[2-Carboxy-3-(2-naphthyl)propyl]-2-oxo-5-(2-phenethyl)-1,2-dihydropyrimidin-4-yl]piperidin-4-yl}amino)-1H-benzimidazol-1-ium trifluoroacetate ESI-MS [M+H]⁺: 627.5 (MW: 626.7)

### Example XVII

2-[(1-{5-Benzyl-1-[2-carboxy-3-(2-naphthyl)propyl]-2-oxo-1,2-dihydropyrimidin-4-yl)-piperdin-4-yl)amino]-1H-benzimidazol-1-ium trifluoroacetate

ESI-MS [M+H]⁺: 613 (MW: 612.7)

### Example XVIII

2-[(1-{1-[2-Carboxy-3-(2-naphthyl)propyl]-2-oxo-5-phenyl-1,2-dihydropyrimidin-4-yl}-piperdin-4-yl)amino]-1H-benzimidazol-1-ium trifluoroacetate

ESI-MS [M+H]⁺: 599 (MW: 598.7)

### Example XIX

2-[(1-(1-[2-Carboxy-3-(2-naphthyl)propyl]-2-oxo-5-propyl-1,2-dihydropyrimidin-4-yl}-piperdin-4-yl)amino]-1H-benzimidazol-1-ium trifluoroacetate

ESI-MS [M+H]⁺: 565 (MW: 564.7)

### Example XX

2-[(1-{1-[2-Carboxy-3-(2-naphthyl)propyl]-2-oxo-5-tetrahydro-2H-pyran-4-yl-1,2-dihydropyrimidin-4-yl}piperdin-4-yl)amino]-1H-benzimidazol-1-ium trifluoroacetate

ESI-MS [M+H]⁺: 607 (MW: 606.7)

II. Biological and Pharmacological Examples

Example 1

Integrin αᵥβ₃ Assay

Integrin αᵥβ₃ antagonists were identified and assessed using a test system based on competition between the natural integrin αᵥβ₃ ligand vitronectin and the test substance for binding to integrin αᵥβ₃ bound to a solid phase.

Procedure:

coat microtiter plates with 250 ng/ml integrin-αᵥβ₃ in 0.05 M NaHCO₃ pH 9.2; 0.1 ml/well;

saturate with 1% milk powder/assay buffer; 0.3 ml/well; 0.5 h/RT

wash 3× with 0.05% Tween 20/assay buffer

Test substance in 0.1% milk powder/assay buffer, 50 μl/well+0 μg/ml or 2 μg/ml human vitronectin (Boehringer Ingelheim T007) in 0.1% milk powder/assay buffer, 50 μl/well; 1 h/RT

wash 3× with 0.05% Tween 20/assay buffer

1 μg/ml anti human vitronectin antibody coupled to peroxidase (Kordia SAVN-APHRP) in 0.1%: milk powder/assay buffer; 0.1 ml/well; 1 h/RT

wash 3× with 0.05% Tween 20/assay buffer

0.1 ml/well peroxidase substrate

stop reaction with 0.1 ml/well 2 M H₂SO₄

measure the absorption at 450 nm

Integrin αᵥβ₃: human placenta is solubilized with Nonidet, and integrin αᵥβ₃ is affinity-purified on a GRGDSPK matrix (elution with EDTA). Contamination by integrin α_{IIb}β₃ and human serum albumin, as well as the detergent and EDTA, are removed by anion exchange chromatography.

Assay buffer: 50 mM tris pH 7.5; 100 mM NaCl; 1 mM CaCl₂; 1 mM MgCl₂; 10 ΞM MnCl₂

Peroxidase substrate: mix 0.1 ml of TMB solution (42 mM TMB In DMSO) and 10 ml of substrate buffer (0.1 M Na acetate pH 4.9) and then add 14.7 pi of 3% H₂O₂.

Various dilutions of the test substances are employed in the assay, and the IC₅₀ values are determined (concentration of the ligand at which 50% of the ligand is displaced). Compound I from the examples showed the best result in this.

### Example 2

Integrin α_{IIb}β₃ Assay

The assay is based on competition between the natural integrin α_{IIb}β₃ ligand fibrinogen and the test substance for binding to integrin α_{IIb}β₃.

Procedure:

coat microtiter plates with 10 μg/ml fibrinogen (Calbiochem 341578) in 0.05 M NaHCO₃ pH 9.2; 0.1 ml/well;

saturate with 1% BSA/PBS; 0.3 ml/well; 30 min/RT

wash 3× with 0.05% Tween 20/PBS

Test substance in 0.1% BSA/PBS; 50 μl/well+200 μg/ml integrin α_{IIb}β₃ (Kordia) in 0.1% BSA/PBS; 50 μl/well; 2 to 4 h/RT

wash 3× as above

biotinylated anti-integrin α_{IIb}β₃ antibody (Dianova CBL 130 B); 1:1000 in 0.1% BSA/PBS; 0.1 ml/well; 2 to 4 h/RT

wash 3× as above

streptavidin-peroxidase complex (B.M. 1089153) 1:10,000 in 0.1% BSA/PBS;

0.1 ml/well; 30 min/RT

wash 3× as above

0.1 ml/well peroxidase substrate

stop reaction with 0.1 ml/well 2 M H₂SO₄

measure the absorption at 450 nm

Peroxidase substrate: mix 0.1 ml of TMB solution (42 mM TMB in DMSO) and 10 ml of substrate buffer (0.1 M Na acetate pH 4.9), then add 14.7.0 of 3% H₂O₂

Various dilutions of the test substances are employed in the assay, and the IC₅₀ values are determined (concentration of the antagonist at which 50% of the ligand is displaced). The selectivity of the substances can be determined by comparing the IC₅₀ values in the integrin α_{IIb}β₃ and integrin αᵥβ₃ assays.

### Example 3

CAM Assay

The CAM (chorioallantoic membrane) assay is a generally accepted model for assessing the in vivo activity of integrin αᵥβ₃ antagonists. It is based on the inhibition of angiogenesis and neovascularization of tumor tissue (Am. J. Pathol. 1975, 79, 597-618; Cancer Res. 1980, 40, 2300-2309; Nature 1987, 329, 630). The procedure is analogous to the -prior art. The growth of the chicken embryonic blood vessels and of the transplanted tumor tissue can readily be followed and assessed.

### Example 4

Rabbit Eye Assay

The inhibition of angiogenesis and neovascularization in the presence of integrin αᵥβ₃ antagonists can be followed and assessed in this in vivo model in analogy to Example 3. The model is generally accepted and is based on the growth of blood vessels starting from the edge into the cornea of the rabbit eye (Proc. Natl. Acad. Sci. USA. 1994, 91, 40824085; Science 1976, 193, 70-72). The procedure is analogous to the prior art.

### Example 5

Investigation of the Pharmacokinetic Properties in the CACO Model

The tests were carried out as described by W. Rubas and M. Cromwell in Advanced Drug Delivery Reviews 23 (1997) 157-162, J. Handler, N. Green and R. Steele in Methods in Enzymology 171 (1989) 736744 and K. Dharmsathaphom and J. Madara in Methods in Enzymology 192 (1990) 354-370.

We claim:

1. A compound of the formula I
B-G-I
where B, G and L have the following meanings:
L is a structural element of the formula I_{L} 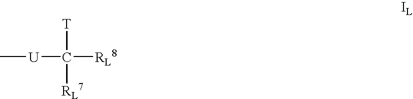
where
T is a COOH group or a radical which can be hydrolyzed to COOH and
—U— iS —(CR_{L}¹R_{L}²)ₐ—(V_{L})_{b}—(W_{L})_{d}—(W_{L})_{d}—(CR_{L}⁵R_{L}⁵)ₑ—(Xᵢ)_{f}
where
a, c, e
are, independently of one another, 0, 1, 2 or 3,
b, d, f
are, independently of one another, 0 or 1,
R_{L}¹, R_{L}², R_{L}³, R_{L}⁴, R_{L}⁵, R_{L}⁶
are, independently of one another, hydrogen, halogen, a hydroxyl group, a branched or unbranched, optionally substituted C₁-C₆-alkyl, C6-alkenyl, C₂-C₆-alkynyl or C₁-C₆-alkylene-C₃-C₇-cycloalkyl radical, a radical —(CH₂)_{w}—(Y_{L})_{y}—R_{L}⁹, an optionally substituted C₃-C₇-cycloalkyl, aryl, arylalkyl, hetaryl or hetarylalkyl radical or, in each case independently of one another, two radicals R_{L}¹ and R_{L}² or R_{L}³ and R_{L}⁴ or R_{L}⁵ and R_{L}⁶ together are a 3 to 7-membered, optionally substituted, saturated or unsaturated carbocyclic or heterocyclic system which may contain up to three heteroatoms from the group of O, N or S,
w is 0, 1, 2, 3 or 4,
y is 0 or 1
Y_{L} is —CO—, —CO—N(R_{y}¹)—, —N(R_{y}¹)—CO—, —N(R_{y}¹)—CO—N(R_{y}¹*)—, —N(R_{y}¹)—CO—O, —O—, —S—, —SO₂—, —SO₂—N(R_{y}¹)—, —SO₂—O—, —CO—O—, —O—CO—, —O—CO—N(R_{y}¹N(R_{y}¹) or —N(R_{y}¹)—SO₂—,
R_{y}^{1,} R_{y}^{1*}
are, independently of one another, hydrogen, a branched or unbranched, optionally substituted C₁-C₆-alkyl, C₀-C₆-alkoxyalkyl, C₂-C₆-alkenyl, C₃-C₁₂-alkynyl, CO-C₁-C₆-alkyl, CO—O—CO—C-alkyl or SO₂-C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂alkylene-aryl radical,
R_{L}^{Y}, R_{L}⁸
are, independently of one another, hydrogen, halogen, a hydroxyl group, a branched or unbranched, optionally substituted C₁-C₆-alkyl, C₂-C₆-alkenyl, C₂-C₆-alkynyl or C₁-C₆-alkylene-C₃-C₇-cycloalkyl radical, an optionally substituted —(CH₂)_{w}—R_{L}^{9*} radical, an optionally substituted C₃-C₇-cycloalkyl, aryl, arylalkyl, hetaryl or hetarylalkyl radical or the radicals R_{L}⁷ and R_{L}⁸ together are a 3 to 7-membered, optionally substituted, saturated or unsaturated carbocyclic or heterocyclic system which may contain up to three heteroatoms from the group of O, N or S,
R_{L}⁹ is hydrogen, or a hydroxyl group, CN, halogen, a branched or unbranched, optionally substituted C₁-C₅-alkyl radical, an optionally substituted C₃-C₇-cycloalkyl, aryl, heteroaryl or arylalkyl radical, an optionally C₁-C₄-alkyl or aryl-substituted C₂-C₆-alkynyl or C₂-C₆-alkenyl radical, an optionally substituted C₆-C₁₂-bicycloalkyl, C₁-C₆-alkylene-C₆-C₁₂-cycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system to be optionally substituted, or for another, optionally substituted, saturated, unsaturated or aromatic cyclic system to be fused to this cyclic system, or the radical R_{L}⁹ forms together with R_{y}¹ or R_{y}¹* a saturated or unsaturated C₃-C₇-heterocycle which may optionally contain up to two further heteroatoms selected from the group of O, S or N,
R_{L}^{9*} is hydrogen, or a hydroxyl group, CN, halogen, a branched or unbranched, optionally substituted C₁-C₆-alkyl radical, an optionally substituted C₃-C₇-cycloalkyl, aryl, heteroaryl or arylalkyl radical, an optionally C₁-C₄-alkyl- or aryl-substituted C₂-C₆-alkynyl or C₂-C₆-alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-c₂₀-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system to be optionally substituted, or for another, optionally substituted, saturated, unsaturated or aromatic cyclic system to be fused to this cyclic system,
W_{L} is an optionally substituted 4- to 11-membered mono- or polycyclic aliphatic or aromatic hydrocarbon which may contain up to 6 double bonds and up to 6 heteroatoms selected from the group of N, O, S,
V_{l,} X_{L}
are, independently of one another, —CO—, —CO—NR_{L}¹⁰—, —NR_{L}¹⁰—CO—, —S—, —SO—, —SO₂—, —SO₂—NR_{L}¹⁰—, —NR_{L}¹⁰—SO₂—, —CS—, —CS—, —CS—NR_{L}¹⁰—, —NR_{L}^{10—CS—, —CS—O—, —O—CS—, —CO—O—, —O—CO—, —O—, ethynylene, —CHR}_{L}¹¹—O—CHR_{L}¹²—, —C(═CR_{L}¹¹R_{L}¹²)—, —CR_{L}¹¹═CR_{L}¹²—, —CR_{L}¹¹(OR_{L}¹³)—CHR_{L}¹²—, —CHR_{L}¹¹—CR_{L}¹²(OR_{L}¹³)—, —CH(NR_{L}¹⁴—SO₂R¹⁵)—, —CH(NR_{L}¹⁴—CO—R_{L}¹⁵)—, —CH(NR_{L}¹⁴—CO—OR_{L}¹⁶)—, CH(NR_{L}¹⁴—CO—NR_{L}¹⁴R_{L}¹⁵)—, —CH(CO—R_{L}¹⁵)—, —CH(CO—OR_{L}¹⁶)— or CH(CO—NR_{L}¹⁴R_{L}¹⁵)—,
L a structural element of the formula I_{L} 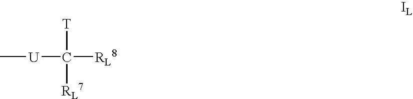
where
T is a COOH group or a radical which can be hydrolyzed to COOH and
—U— is —(CR_{L}¹R_{L}²)ₐ—(V_{L})_{b}—(CR_{L}³R_{L}⁴)_{c}—(W_{L})_{d}—(CR_{L}⁶R_{L}⁶)ₑ—(X_{L})_{f}
where
a, c, e
are, independently of one another, 0, 1, 2 or 3,
b, d, f
are, independently of one another, 0 or 1,
R_{L}¹, R_{L}², R_{L}⁹, R_{L}⁴, R_{L}⁵, RL⁶
are, independently of one another, hydrogen, halogen, a hydroxyl group, a branched or unbranched, optionally substituted C₁-C₆-alkyl, C₂-C₆-alkenyl, C₂-C₆-alkynyl or C₁-C₆-alkylene-C₃-C₇-cycloalkyl radical, a radical —(CH₂)_{w}—(Y_{L})_{y}—R_{L}⁹, an optionally substituted C₃-C₇-cycloalkyl, aryl, arylalkyl, hetaryl or hetarylalkyl radical or, in each case independently of one another, two radicals R_{L}¹ and R_{L}² or R_{L}³ and R_{L}⁴ or R_{L}⁵ and R_{L}⁶ together are a 3 to 7-membered, optionally substituted, saturated or unsaturated carbocyclic or heterocyclic system which may contain up to three heteroatoms from the group of O, N or S,
w is 0, 1, 2, 3 or 4,
y is 0 or 1
Y_{L} is —CO—, —CO—N(R_{Y}¹)—, —N(R_{Y}¹)—CO—, —N(R_{Y}¹)—CO—N(R_{Y}¹*)—, —N(R_{Y}¹)—CO—O—, —O—, —S—, —SO₂—, —SO₂—N(R_{Y}¹), —SO₂—O—, —CO—O—, —O—CO—, —O—CO—N(R_{y}¹)—, —N(R_{Y}¹)— or —N(R_{Y}¹)—SO₂—,
R_{Y}^{1,} R_{Y}¹*
are, independently of one another, hydrogen, a branched or unbranched, optionally substituted C₁-C₆-alkyl, C₁-C₆-alkoxyalkyl, C₂-C₆-alkenyl, C₃-C₁₂-alkynyl, CO-C₁-C₆-alkyl, CO—O-C₁-C₆-alkyl or SO₂C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂-alkylene-aryl radical,
R_{L}⁷, R_{L}⁸
are, independenty of one another, hydrogen, halogen, a hydroxyl group, a branched or unbranched, optionally substituted C₁-C₆-alkyl, C₂-C₆-alkenyl, C₂-C₆-alkynyl or C₁-C₆-alkylene-C₃-C₇-cycloalkyl radical, an optionally substituted —(CH₂)_{w}—R_{L}^{9*} radical, an optionally substituted C₃-C₇-cycloalkyl, aryl, arylalkyl, hetaryl or hetarylalkyl radical or the radicals R_{L}⁷ and R_{L}⁸ together are a 3- to 7-membered, optionally substituted, saturated or unsaturated carbocyclic or heterocyclic system which may contain up to three heteroatoms from the group of O, N or S,
R_{L}⁹ is hydrogen, or a hydroxyl group, CN, halogen, a branched or unbranched, optionally substituted C₁-C₆-alkyl radical, an optionally substituted C₃-C₇-cycloalkyl, aryl, heteroaryl or arylalkyl radical, an optionally C₁-C₄-alkyl- or aryl-substituted C₂-C₆-alkynyl or C₂-C₆-alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system to be optionally substituted, or for another, optionally substituted, saturated, unsaturated or aromatic cyclic system to be fused to this cyclic system, or the radical R_{L}⁹ forms together with R_{y}¹ or R_{y}¹* a saturated or unsaturated C₃-C₇-heterocycle which may optionally contain up to two further heteroatoms selected from the group of O, S or N,
R_{L}^{9*} is hydrogen, or a hydroxyl group, CN, halogen, a branched or unbranched, optionally substituted C₁-C₆-alkyl radical, an optionally substituted C₃-C₇-cycloalkyl, aryl, heteroaryl or arylalkyl radical, an optionally C₁C₄-alkyl or aryl-substituted C₂-C₆-alkynyl or C₂-C₆-alkenyl radical, an optionally substituted C₆C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system to be optionally substituted, or for another, optionally substituted, saturated, unsaturated or aromatic cyclic system to be fused to this cyclic system,
W_{L} is an optionally substituted 4- to 11-membered mono- or polycyclic aliphatic or aromatic hydrocarbon which may contain up to 6 double bonds and up to 6 heteroatoms selected from the group of N, O, S,
V_{L}, X_{L}.
are, independently of one another, —CO—, CO—NR_{L}¹⁰—, —NR_{L}¹⁰—CO—, —S—, —SO—, —SO₂—, —SO₂—NR_{L}¹⁰—, —NR_{L}¹⁰—SO₂—, —CS—, —CS—NR_{L}¹⁰—, —NR_{L}¹⁰—CS—, —CS—O—, —O—CS—, —CO—O—, —O—CO—, —O—, ethynylene, —CHR_{L}¹¹—O—CHR_{L}¹²—, —C(═CR_{L}¹¹R_{L}¹²)—, —CR_{L}¹¹═CR_{L}¹²—, —CR_{L}¹¹(OR_{L}¹³)—CHR_{L}¹²—, —CHR_{L}¹¹—CR_{L}¹²(OR_{L}¹³)—, —CH(NR_{L}¹⁴—SO₂—R_{L}¹⁵)—, —CH(NR_{L}¹⁴—CO—R_{L}¹⁵)—, —CH(NR_{L}¹⁴—CO—OR_{L}¹⁶)—, CH(NR_{L}¹⁴—CO—NR_{L}^{14′}R_{L}¹⁵)—, —CH(CO—R_{L}¹⁵)—, —CH(CO—OR_{L}¹⁶)— or CH(CO—NR_{L}¹⁴R_{L}¹⁵)—,
R_{L}¹⁰ is hydrogen, a branched or unbranched, optionally substituted C₁-C₆-alkyl, C₁-C₆-alkoxyalkyl, C₂-C₆alkenyl, C₃-C₁₂-alkynyl, CO-C₁-C₆-alkyl, CO—O-C₁-C₆-alkyl or SO₂-C₁-C₆-alkyl radical or an optionally substituted C₃-C₇-cycloalkyl, CO—O-alkylene-aryl, CO-alkylene-aryl, arylalkyl, CO-aryl, SO₂-aryl, hetaryl, CO-hetaryl, hetarylalkyl or SO₂-alkylene-aryl radical, or R_{L}¹⁰ and a radical selected from the group of R_{L}¹, R_{L}¹, R_{L}³, R_{L}⁴, R_{L}⁵ or R_{L}⁶ together are an optionally substituted 4- to 8-membered heterocycle which may contain up to five identical or different heteroatoms O, N or S,
R_{L}^{11,} R_{L}¹²
are, independently of one another, hydrogen, a hydroxyl group, a branched or unbranched, optionally substituted C₁-C₆-alkyl, C₁-C₄-alkoxy, C₂-C₆-alkenyl, C₂-C₆alkynyl or alkylene-cycloalkyl radical or an optionally substituted C₃-C₇-cycloalkyl, aryl, arylalkyl, hetaryl or hetarylalkyl radical,
R_{L}¹³ is hydrogen, a branched or unbranched, optionally substituted C₁-C₆-alkyl, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,
R_{L}¹⁴, R_{L}¹⁴,
are, independently of one another, hydrogen, a branched or unbranched, optionally substituted C₁-C₆-alkyl, C₁-C₆-alkoxyalkyl, C₂-C₆-alkenyl, C₃-C₁₂-alkynyl, CO-C₁-C₆-alkyl, CO—O-C₁-C₆-alkyl, C₁-C₆-alkylene-C₃-C₇-cycloalkyl, or SO₂-C₁-C₆-alkyl radical or an optionally substituted C₃-C₇-cycloalkyl, CO—O-alkylene-aryl, CO-alkylene-aryl, aryl, CO-aryl, SO₂-aryl, hetaryl, CO-hetaryl, hetarylalkyl, arylalkyl or SO₂-alkylene-aryl radical,
R_{L}¹⁵ is a branched or unbranched, optionally substituted C₁-C₆-alkyl, alkoxyalkyl, C₁-C₆-alkylene-C₃-C₇-cycloalkyl radical, C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, a C₃-C₇-cycloalkyl, aryl, arylalkyl, hetarylalkyl or 3 to 8-membered, saturated, unsaturated or aromatic heterocyclic radical which may be substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for this cyclic system to be optionally substituted, or for another, optionally substituted, saturated, unsaturated or aromatic cyclic system to be fused to this cyclic system, or the radical R_{L}¹⁵ forms together with R_{L}¹⁴ or R_{L}¹⁴* a saturated or unsaturated C₃-C₇ heterocycle which may optionally contain up to two further heteroatoms selected from the group of O. S or N, and
R_{L}¹⁶ is a branched or unbranched, optionally substituted C₁-C₆-alkyl, alkoxyalkyl or C₁-C₆-alkylene-C₃-C₇-cycloalkyl radical or an optionally substituted C₃-C₇-cycloalkyl, aryl, arylalkyl, hetaryl or hetarylalkyl radical,
G is a structural element of the formula I_{G} 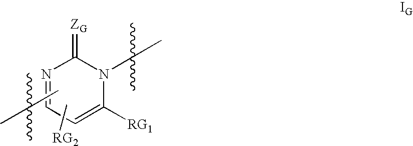
where
the structural element G can be incorporated in both orientations, and
Z_{G} is oxygen, sulfur or NR_{G}³,
R_{G}¹, R_{G}²
are, independently of one another, hydrogen, CN, NO_{2,} halogen, a branched or unbranched, optionally substituted C₁-C₆-alkyl, C₂-C₆-alkenyl or C₂-C₆-alkynyl radical, a branched or unbranched, optionally substituted C₁-C₄alkylene-OR_{G}⁴, C₁-C₄-alkylene-CO—OR_{G}⁴, C₁-C₄-alkylene-CO-R_{G}⁴, C₁-C₄-alkylene-SO₂-NR_{G}⁵R_{G}⁶, C₁-C₄-alkylene-CO—NR_{G}⁵R_{G}⁸, C₁-C₄-alkylene-NR_{G}⁵R_{G}⁶ or C₁-C₄-alkylene-SR_{G}⁴ radical, an optionally substituted C₃-C₇-cycloalkyl, C₁-C₄-alkylene-C₃-C₇-cycloalkyl, C₁-C₄-alkylene-C₃-C₇-heterocycloalkyl or C₁-C₄-alkylene-C₃-C₇-heterocyclo-alkenyl radical, an optionally substituted aryl, arylalkyl, hetaryl or hetarylalkyl radical, an —S—R_{G}⁴, —O—R_{G}⁴, —SO—R_{G}⁴, —SO₂—R_{G}⁴, —CO—OR_{G}⁴, —O—CO—R_{G}⁴, —O—CO—NR_{G}⁵R_{G}⁶, —SO₂—NR_{G}⁵R_{G}⁶, —CO—NR_{G}⁵R_{G}⁸, —NR_{G}⁵R_{G}⁶, CO—R_{G}⁴ radical, or R_{G}¹ and R_{G}² together are an optionally substituted, saturated, unsaturated or aromatic 3- to 9-membered carbocyclic, polycarbocyclic, heterocyclic or polyheterocyclic system which may contain up to 4 heteroatoms selected from the group of O, N, S,
R_{G}³ is hydrogen, a hydroxyl group, CN, a branched or unbranched, optionally substituted C₁-C₆-alkyl or C₁-C₄-alkoxy radical or an optionally substituted C₃-C₇-cycloalkyl, —O-C₃-C₇-cycloalkyl radical, aryl, aryl, arylalkyl or —O-alkylene-aryl radical,
R_{G}⁴ is hydrogen, a branched or unbranched, optionally substituted C₁-C₈-alkyl, C₂-C₆-alkenyl, C₂-C₆-alkynyl, C₁-C₅-alkylene-C₁-C₄alkoxy, mono- and bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cloalkyl, arylalkyl, C₁-C₄-alkylene-heterocycloalkyl, C₁-C₄-alkylene heterocycloalkenyl or hetarylalkyl radical,
R_{G}⁵, R_{G}⁶
are, independently of one another, hydrogen, a branched or unbranched, optionally substituted C₁-C₈-alkyl, C₂-C₆-alkenyl, C₂-C₆-alkynyl, C₁-C₆-alkylene-C₁-C₄-alkoxy, mono- and bisalkylaminoalkylene or acylamino-alkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄alkylene-heterocycloalkyl, C₁-C₄-alkylene-heterocycloalkenyl or hetarylalkyl radical, or an —SO₂—R_{G}⁴, —CO—OR_{G}⁴, —CO—NR_{G}⁴R_{G}⁴* or —CO—R_{G}⁴ radical, and
R_{L}¹⁰ is hydrogen, a branched or unbranched, optionally substituted C₁-C₆-alkyl, C₁-C₆-alkoxyalkyl, C₂-C₆-alkenyl, C₃-C₁₂-alkynyl, CO-C₁-C₆-alkyl, CO—O-C₁-C₆-alkyl or SO₂-C₁-C₆-alkyl radical or an optionally substituted C₃-C₇-cycloalkyl, CO—O-alkylene-aryl, CO-alkylene-aryl, aryaalkyl, CO-aryl, SO₂-aryl, hetaryl, CO—O-hetaryl, hetarylalkyl or SO₂-alkylene-aryl radical, or R_{L}¹⁰ and a radical selected from the group of R_{L}¹, R_{L}², R_{L}³, R_{L}⁴, R_{L}⁵ or R_{L}⁶ together are an optionally substituted 4- to 8-membered heterocycle which may contain up to five identical or different heteroatoms O, N or S,
R_{L}^{11,} R_{L}¹²
are, independently of one another, hydrogen, a hydroxyl group, a branched or unbranched, optionally substituted C₁-C₆-alkyl, C₁-C₄-alkoxy, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,
R_{L}¹³ is hydrogen, a branched or unbranched, optionally substituted C₁-C₆-alkyl, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,
R_{L}¹⁴, R_{L}^{14,}
are, independently of one another, hydrogen, a branched or unbranched, optionally substituted C₁-C₆-alkyl, C₁-C₆-alkoxyalkyl, C₂-C₆-alkenyl, C₃-C₁₂-alkynyl, CO-C₁-C₆-alkyl, CO—O-C₁-C₆-alkyl, C₁-C₆-alkylene C₃-C₇-cycloalkyl, or SO₂-C₁-C₆-alkyl radical or an optionally substituted C₃-C₇-cycloalkyl, CO—O-alkylene-aryl, CO-alkylene-aryl, aryl, CO-aryl, SO₂-aryl, hetaryl, CO-hetaryl, hetarylalkyl, arylalkyl or SO₂-alkylene-aryl radical,
R_{L}¹⁵ is a branched or unbranched, optionally substituted C₁-C₆-alkyl, alkoxyalkyl, C₁-C₆-alkylene-C₃-C₇-cycloalkyl radical, C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, a C₃-C₇-cycloalkyl, aryl, arylalkyl, hetarylalkyl or 3- to 8-membered, saturated, unsaturated or aromatic heterocyclic radical which may be substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for this cyclic system to be optionally substituted, or for another, optionally substituted, saturated, unsaturated or aromatic cyclic system to be fused to this cyclic system, or the radical R_{L}¹⁵ forms together with R_{L}¹⁴ or R_{L}¹⁴* a saturated or unsaturated C₃-C₇-heterocycle which may optionally contain up to two further heteroatoms selected from the group of O, S or N, and
R_{L}¹⁶ is a branched or unbranched, optionally substituted C₁-C₆-alkyl, alkoxyalkyl or C₁-C₆-alkylene-C₃-C₇-cycloalkyl radical or an optionally substituted C₃-C₇-cycloalkyl, aryl, arylalkyl, hetaryl or hetarylalkyl radical,
G is a structural element of the formula I_{G} 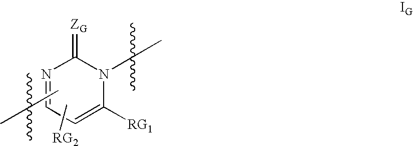
where
the structural element G can be incorporated in both orientations, and
Z_{G} is oxygen, sulfur or NR_{G}³,
R_{G}¹, R_{G}²
are, independently of one another, hydrogen, CN, NO₂ halogen, a branched or unbranched, optionally substituted C₁-C₆-alkyl, C₂-C₆-alkenyl or C₂-C₆-alkynyl radical, a branched or unbranched, optionally substituted C₁-C₄-alkylene-OR_{G}⁴, C₁-C₄-alkylene-CO—OR_{G}⁴, C₁-C₄-alkylene CO—R_{G}⁴, C₁-C₄-alkylene-SO₂—NR_{G}⁵R_{G}⁶, C₁-C₄-alkylene-CO—NR_{G}⁵R_{G}⁶, C₁-C₄-alkylene-NR_{G}⁵R_{G}⁶ or C₁-C₄-alkylene-SR_{G}⁴ radical, an optionally substituted C₃-C₇-cycloalkyl, C₁-C₄-alkylene-C₃-C₇-cycloalkyl, C₁-C₄-alkylene-C₃-C₇-heterocycloalkyl or C₁-C₄-alkylene-C₃-C₇-heterocyclo-alkenyl radical, optionally substituted aryl, arylalkyl, hetaryl or hetarylalkyl radical, an —S—R_{G}⁴, —O—R_{G}⁴, —SO—R_{G}⁴, —SO₂—R_{G}⁴, —CO—OR_{G}⁴, —O—CO—R_{G}⁴, —O—CO—NR_{G}⁵R_{G}⁶, —SO₂—NR_{G}⁵R_{G}⁶, —CO—NR_{G}⁵R_{G}⁶, —NR_{G}⁵R_{G}⁶, CO—R_{G}⁴ radical, or R_{G}¹ and R_{G}² together are an optionally substituted, saturated, unsaturated or aromatic 3- to 9-membered carbocyclic, polycarbocyclic, heterocyclic or polyheterocyclic system which may contain up to 4 heteroatoms selected from the group of O, N, S,
R_{G}³ is hydrogen, a hydroxyl group, CN, a branched or unbranched, optionally substituted C₁-C₆-alkyl or C₁-C₄-alkoxy radical or an optionally substituted C₃-C₇-cycloalkyl, —O-C₃-C₇-cycloalkyl radical, aryl, —O-aryl, arylalkyl or —O-alkylene-aryl radical,
R_{G}⁴ is hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁C₄-alkylene-heterocycloalkyl, C₁-C₄-alkylene-heterocycloalkenyl or hetarylalkyl
R_{G}⁵, R_{G}⁶
are, independently of one another, hydrogen, a branched or unbranched, optionally substituted C₁-C₈-alkyl, C₂-C₆alkenyl, C₂-C₆-alkynyl, C₁-C_{5l -alkylene-C}₁-C₄-alkoxy, mono- and bisalkylaminoalkylene or acylamino-alkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-heterocycloalkyl, C₁-C₄-alkylene-heterocycloalkenyl or hetarylalkyl radical, or an —SO₂—R_{G}⁴, —CO—OR_{G}⁴, —CO—NR_{G}⁴R_{G}⁴* or —CO—R_{G}⁴ radical, and
R_{G}⁴ is an R_{G}⁴ radical independent of R_{G}⁴.
B is a structural element containing at least one atom which can, under physiological conditions, form hydrogen bonds as hydrogen acceptor, where the distance between at least one hydrogen acceptor atom and structural element G along the shortest possible route along the structural element framework is from 4 to 13 atomic linkages, and the physiologically tolerated salts, prodrugs and enantiomerically pure or diastereomerically pure and tautomeric forms.

2. A compound as claimed in claim 1, wherein the structual element of the formula I_{B}
A-E- I_{B}
where A and E have the following meanings:
A a structural element selected from the group:
a 4- to 8-membered monocyclic saturated, unsaturated or aromatic hydrocarbon which may contain up to 4 heteroatoms selected from the group of O, N or S, it being possible in each case, independently of one another, for the ring nitrogen which is present where appropriate or the carbons to be substituted, with the proviso that at least one heteroatom selected from the group of O, N or S is present in the structural element A, or
a 9- to 14-membered polycyclic saturated, unsaturated or aromatic hydrocarbon which may contain up to 6 heteroatoms selected from the group of N, O or S, it being possible in each case, independently of one another, for the ring nitrogen which is present where appropriate or the carbons to be substituted, with the proviso that at least one heteroatom selected from the group of O, N or S is present in the structural element A,
a radical 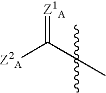
where
Z_{A}¹ is oxygen, sulfur or optionally substituted nitrogen, and
Z_{A}² is optionally substituted nitrogen, oxygen or sulfur, or a radical 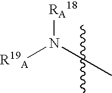
where
R_{A}¹⁸, R_{A}¹⁹
are, independently of one another, hydrogen, a branched or unbranched, optionally substituted C₁-C₈-alkyl, C₂-C₆-alkenyl, C₂-C₆-alkynyl, C₁-C₅-alkylene-C₁-C₄-alkoxy, mono- and bisalkyl-aminoalkylene or acylaminoalkylene radical or an optionally substituted aryl, heterocycloalkyl, heterocycloalkenyl, hetaryl, C₃-C₇-cycloalkyl, C₁-C₄-cycloalkyl, arylalkyl, C₁-C₄-alkylene-heterocycloalkyl, C₁-C₄-alkylene-heterocyclo-alkenyl or hetarylalkyl radical, or an —SO₂—R_{G}⁴, —CO—OR_{G}⁴—CO—NR_{G}⁴R_{G}⁴* or —CO—R_{G}⁴ radical,
and
E a spacer structural element which covalently connects structural element A to structural element G, where the number of atomic linkages along the shortest possible route along the structural element framework E is from 3 to 12.

3. A compound as claimed in claim 2, wherein the structural element A used is a structural element selected from the group of structural elements of the formulae 1_{A}¹ to 1_{A}¹⁹, 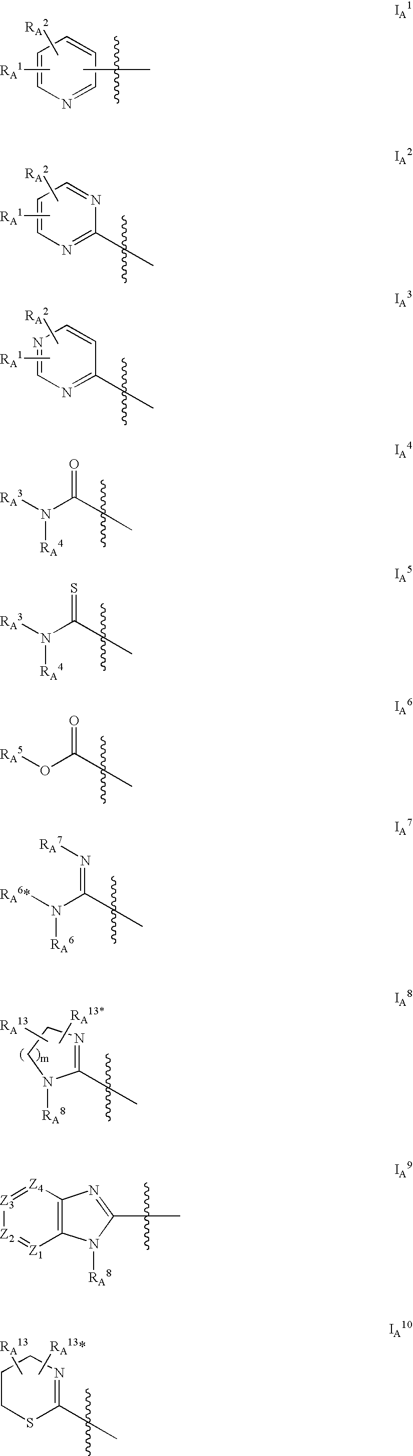 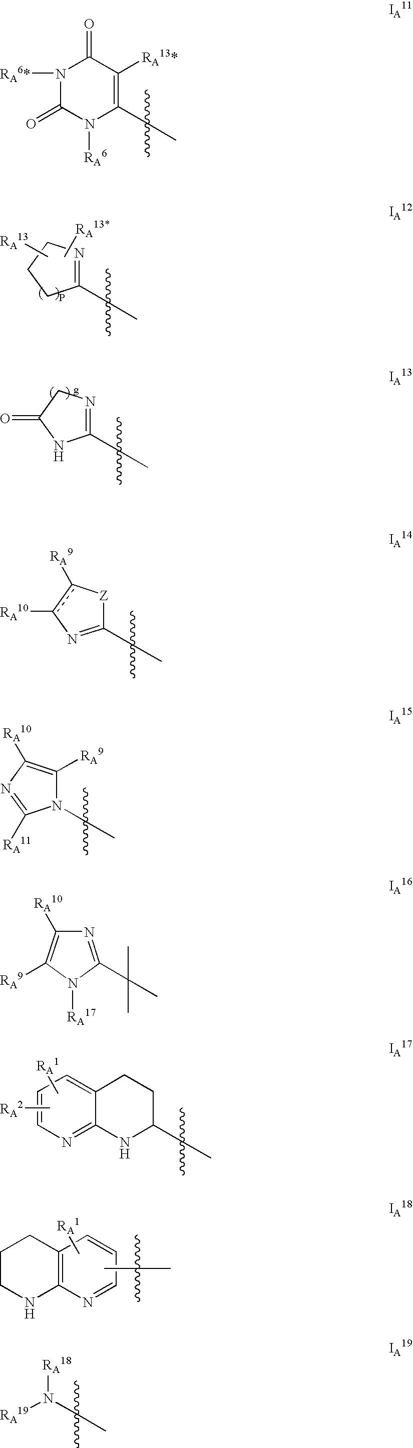
where
m, p, q
are, independently of one another, 1, 2 or 3,
R_{A}¹, R_{A}²
are, independently of one another, hydrogen, CN, halogen, a branched or unbranched, optionally substituted C₁-C₆-alkyl or CO-C₁-C₆-alkyl radical or an optionally substituted aryl, arylalkyl, hetaryl, hetarylalkyl or C₃-C₇-cycloalkyl radical or a CO—O—R_{A}¹⁴, O—R_{A}¹⁴, S—R_{A}¹⁴, NR_{A}¹⁵R_{A}¹⁶, CO—NR_{A}¹⁵R_{A}¹⁶ or SO₂NR_{A}¹⁵R_{A}¹⁸ radical or the two radicals R_{A}¹ and R_{A}² together are a fused, optionally substituted, 5- or 6-membered, unsaturated or aromatic carbocyclic or heterocyclic system which may contain up to three heteroatoms selected from the group of O, N or S,
R_{A}¹³, R_{A}^{13*}
are, independently of one another, hydrogen, CN, halogen, a branched or unbranched, optionally substituted C₁-C₆-alkyl radical or an optionally substituted aryl, arylalkyl, hetaryl, C₃-C₇-cycloalkyl radical or a CO—O—R_{A}¹⁴, O—R_{A}¹⁴, S—R_{A}¹⁴, NR_{A}¹⁵R_{A}¹⁶, SO₂—NR_{A}¹⁵R_{A}¹⁶ or CO—NR_{A}¹⁵R_{A}¹⁶ radical,
where
R_{A}¹⁴ is hydrogen, a branched or unbranched, optionally substituted C₁-C₆-alkyl, alkylene-C₁-C₄-alkoxy, C₂-C₆-alkenyl, C₂-C₆-alkynyl or C₁-C₆-alkylene-C₃-C₇-cycloalkyl radical or an optionally substituted C₃-C₇-cycloalkyl, aryl, arylalkyl, hetaryl or hetarylalkyl radical,
R_{A}¹⁵, R_{A}¹⁶,
are, independently of one another, hydrogen, a branched or unbranched, optionally substituted C₁-C₆-alkyl, CO—C₁-C₆-alkyl, SO₂-C₁-C₆-alkyl, COO-C₁-C₆-alkyl, CO—NH-C₁-C₆-alkyl, arylalkyl, COO-alkylene-aryl, SO₂-alkylene-aryl, CO—NH-alkylene-aryl, CO—NH-alkylene-hetaryl or hetarylalkyl radical or an optionally substituted C₃-C₇-cycloalkyl, aryl, CO-aryl, CO—NH-aryl, SO₂-aryl, hetaryl, CO—NH-hetaryl, or CO-hetaryl radical,
R_{A}^{3,} R_{A}⁴
are, independently of one another, hydrogen, —(CH₂)ₙ—(X_{A})ⱼ—R_{A}¹², or the two radicals together are a 3- to 8-membered, saturated, unsaturated or aromatic N heterocyclic system which may additionally contain two other, identical or different heteroatoms O, N or S, it being possible for the cyclic system optionally to be substituted, or for another, optionally substituted, saturated, unsaturated or aromatic cyclic system to be fused to this cyclic system,
where
n is 0, 1, 2 or 3,
j is 0 or 1,
X_{A} is —CO—, —CO—N(Rₓ¹)—, —N(Rₓ¹)—CO—N(Rₓ¹)—CO—N(Rₓ¹*)—, —N(Rₓ¹)—CO—O—, —O—, —S—, —SO₂—, —SO₂—N(Rₓ¹—, —SO₂—O—, —CO—O—, —O—CO—, —O—CO—N(Rₓ¹)—, —N(Rₓ¹)— or —N(Rₓ¹)—SO₂—,
R_{A}¹² is hydrogen, a branched or unbranched, optionally substituted C₁-C₆-alkyl radical, an optionally C₁-C₄alkyl- or aryl-substituted C₂-C₆-alkynyl or C₂-C₆-alkenyl radical or a 3- to 6-membered, saturated or unsaturated heterocyclic system which is substituted by up to three identical or different radicals and which may contain up to three different or identical heteroatoms O, N, S, or C₃-C₇-cycloalkyl, aryl or heteroaryl radical, it being possible for two radicals together to be a fused, saturated, unsaturated or aromatic carbocyclic or heterocyclic system which may contain up to three different or identical heteroatoms O, N, S, and it being possible for the cyclic system optionally to be substituted or for another, optionally substituted, saturated, unsaturated or aromatic cyclic system to be fused to this cyclic system, or the R_{A}¹² radical forms together with Rₓ¹ or Rₓ¹ a saturated or unsaturated C₃-C₇-heterocycle which may optionally contain up to two other heteroatoms selected from the group of O, S or N,
Rₓ^{1,} Rₓ¹*
are, independently of one another, hydrogen, a branched or unbranched, optionally substituted C₁-C₆-alkyl, C₁-C₆-alkoxyalkyl, C₂-C₆-alkenyl, C₂-C₁₂-alkynyl, CO-C₁-C₆-alkyl, CO—O-C₁-C₆-alkyl or SO₂-C₁-C₆-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂-alkylene-aryl radical,
R_{A}⁵ is a branched or unbranched, optionally substituted C₁-C₆-alkyl, arylalkyl, C₃-C₇-cycloalkyl or C₁-C₄-alkylene-C₃-C₇-cycloalkyl radical or an optionally substituted aryl, hetaryl, heterocycloalkyl or heterocycloalkenyl radical,
R_{A}⁶, R_{A}^{6′}
are hydrogen, a branched or unbranched, optionally substituted C₁-C₄-alkyl, —CO—O-C₁-C₄-alkyl, arylalkyl, —CO—O-alkylene-aryl, —CO—O-allyl, —CO-C₁-C₄-alkyl, —CO-alkylene-aryl, C₃-C₇-cycloalkyl or —CO-allyl radical or the two radicals R_{A}⁶ and R_{A}^{6*} in the structural element I_{A}⁷ together are an optionally substituted, saturated, unsaturated or aromatic heterocyclic system which, in addition to the ring nitrogen, may contain up to two other different or identical heteroatoms O, N, S,
R_{A}⁷ is hydrogen, —OH, —CN, —CONH₂, a branched or unbranched, optionally substituted C₁-C₄-alkyl, C₁-C₄-alkoxy, C₃-C₇-cycloalkyl or —O—CO-C₁-C₄-alkyl radical or an optionally substituted arylalkyl, —O-alkylene-aryl, —O—CO-aryl, —O—CO-alkylene-aryl or —O—CO-allyl radical, or the two radicals R_{A}⁶ and R_{A}⁷ together are an optionally substituted, unsaturated or aromatic heterocyclic system which, in addition to the ring nitrogen, may contain up to two other different or identical heteroatoms O, N, S,
R_{A}⁸ is hydrogen, a branched or unbranched, optionally substituted C₁-C₄-alkyl, CO-C₁-C₄-alkyl, SO₂-C₁-C₄-alkyl or CO—-C₁-C₄-alkyl radical or an optionally substituted aryl, CO-aryl, SO₂-aryl, CO—O-aryl, CO-alkylene-aryl, SO₂-alkylene-aryl, CO—O-alkylene-aryl or alkylene-aryl radical,
R_{A}⁹, R_{A}¹⁰
are, independently of one another, hydrogen, —CN, halogen, a branched or unbranched, optionally substituted C₁-C₆-alkyl radical or an optionally substituted aryl, arylalkyl, hetaryl, C₃-C₇-cycloalkyl radical or a CO—O—R_{A}¹⁴, O—R_{A}¹⁴, S—R_{A}¹⁴, NR_{A}¹⁵R_{A}¹⁶, SO₂-NR_{A}¹⁵R_{A}¹⁶ or CO—NR_{A}¹⁵R_{A}¹⁶ radical, or the two radicals R_{A}⁹ and R_{A}¹⁰ in the structural element I_{A}¹⁴ together are a 5- to 7-membered saturated, unsaturated or aromatic carbocyclic or heterocyclic system which may contain up to three different or identical heteroatoms O, N, S and is optionally substituted by up to three identical or different radicals,
R_{A}¹¹ is hydrogen, —CN, halogen, a branched or unbranched, optionally substituted C₁-C₆-alkyl radical or an optionally substituted aryl, arylalkyl, hetaryl, C₃-C₇-cycloalkyl radical or a CO—O—R_{A}¹⁴, O—R_{A}¹⁴, S—R_{A}¹⁴, NR_{A}¹⁵R_{A}¹⁶, SO₂NR_{A}¹⁵R_{A}¹⁶ or CO—NR_{A}¹⁵R_{A}¹⁶ radical,
R_{A}¹⁷ is hydrogen or the two radicals R_{A}⁹ and R_{A}¹⁷ in the structural element I_{A}¹⁶ together are a 5- to 7-membered saturated, unsaturated or aromatic heterocycle which, in addition to the ring nitrogen, may contain up to three different or identical heteroatoms O, N, S and is optionally substituted by up to three identical or different radicals,
R_{A}¹⁸, R_{A}¹⁹
are, independently of one another, hydrogen, a branched or unbranched, optionally substituted C₁-₈-alkyl, C₂-C₆-alkenyl, C₂-C₆alkynyl-, C₁-C₅-alkylene-C₁-C₄-alkoxy, mono- and bisalkylaminoalkylene or acylamino-alkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-heterocycloalkyl, C₁-C₄-alkylene-heterocycloalkenyl or hetarylalkyl radical, or an —SO₂—R_{G}⁴, —CO—OR_{G}⁴, —CO—NR_{G}⁴R_{G}⁴* or —CO—R_{G}⁴ radical,
Z^{1,} Z^{2,} Z³, Z⁴
are, independently of one another, nitrogen, C—H, C-halogen or a branched or unbranched, optionally substituted C-C₁-C₄-alkyl or C-C₁-C₄-alkoxy radical,
Z⁵ is NR_{A}⁵, oxygen or sulfur.

4. A compound as claimed in claim 2, wherein the spacer structural element E is a structural element of the formula l_{E}.
—(NR_{E}¹)ₗ-E₁-(U_{E})ₕ— I_{E}
where
U_{E} is oxygen, sulfur or NR_{E}²,
h is 0 or 1,
i is 0 or 1,
R_{E}¹, R_{E}²
are, independently of one another, hydrogen, a branched or branched, optionally substituted C₁-C₆-alkyl, C₁-C₆-alkoxyalkyl, C₂-C₆-alkenyl, C₂-C₁₂-alkynyl, CO—C₁-C₆-alkyl, CO—O-C₁-C₆-alkyl, CO—NH-C₁-C₆-alkoxyalkyl, CO—NH-C₁-C₆-alkyl or SO₂-C₁-C₆-alkyl radical or an optionally substituted hetaryl, arylalkyl, C₃-C₇-cycloalkyl, CO—O-alkylene-aryl, CO—NH-alkylene-aryl, CO-alkylene-aryl, CO-aryl, CO—NH-aryl, SO₂-aryl, CO-hetaryl, SO₂-alkylene-aryl, SO₂-hetaryl or SO₂-alkylene-hetaryl radical,
E₁ is a structural element of the formula I_{E1}
—(CR_{E}³R_{E}⁴)ₖ₁-(L_{E})ₖ₂—(CR_{E}⁵R_{E}⁶)ₖ₃-(Q_{E})ₖ₄—(CR_{E}⁷R_{E}⁸)ₖ₆—(T_{E})ₖ₆—(CR_{E}⁹R_{E}¹⁰)ₖ₇— I_{E1}
where
k2, k4, k6
are 0 or 1,
k1, k3, k5, k7
are 0, 1 or 2,
R_{E}³, R_{E}⁴, R_{E}⁵, R_{E}⁶, R_{E}⁷, R_{E}⁸, R_{E}⁹, R_{E}¹⁰
are, independently of one another, hydrogen, halogen, a hydroxyl group, a branched-or unbranched, optionally substituted C₁-C₆-alkyl, C₂-C₈-alkenyl, C₂-C₆-alkynyl or alkylene-cycloalkyl radical, a —(CH₂)ₓ—(Y_{E})_{z}R_{E}¹¹ radical, an optionally substituted C₃-C₇-cycloalkyl, aryl, arylalkyl, hetaryl or hetarylalkyl radical or, independently of one another, in each case two radicals R_{E}³ and R_{E}⁴ or R_{E}⁵ and R_{E}⁶ or R_{E}⁷ and R_{E}⁸ or R_{E}⁹ and R_{E}¹⁰ together are a 3- to 7-membered, optionally substituted, saturated or unsaturated carbo- or heterocyclic system which may contain up to three heteroatoms from the group of O, N or S,
x is 0, 1, 2, 3 or 4,
z is 0 or 1,
Y_{E} is —CO—, —CO—N(R_{y}²)—, —N(R_{y}²)—CO—, —N(R_{y}²)—CO—N(R_{y}²)—, —N(R_{y}²)—CO—O—, —S—, —SO₂—, —SO₂—N(R_{y}²)—, —SO₂—O—, —CO—CO—, —O—CO—, —O—CO—N(R_{y}²⁾—, —N(R_{y}²)— or —N(R_{y}²)—SO₂—,
R_{y}^{2,} R_{y}²*
are, independently of one another, hydrogen, a branched or unbranched, optionally substituted C₁-C₆-alkyl, C₂-C₆-alkenyl, C₂-C₈-alkynyl, CO—C₁-C₆-alkyl, CO—O-C₁-C₆-alkyl or SO₂-C₁-C₆-alkyl radical or an optionally substituted hetaryl, hetarylalkyl, arylalkyl, C₃-C₇-cycloalkyl, CO—O-alkylene-aryl, CO-alkylene-aryl, CO-aryl, SO₂-aryl, CO-hetaryl or SO₂-alkylene-aryl radical,
R_{E}¹¹ is hydrogen, a hydroxyl group, CN, halogen, a branched or unbranched, optionally substituted C₁-C₆-alkyl radical, an optionally substituted C₃-C₇-cycloalkyl, aryl, hetaryl or arylalkyl radical, an optionally C₁-C₄-alkyl- or aryl-substituted C₂-C₆-alkynyl or C₁-C₆-alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₁-C₂₁-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for the cyclic system optionally to be substituted or for another, optionally substituted, saturated, unsaturated or aromatic cyclic system to be fused to this cyclic system, or the R_{E}¹¹ radical forms together with R_{y}² or R_{y}²* a saturated or unsaturated C₃-C₇-heterocyclic system which may optionally contain up to two other heteroatoms selected from the group of O, S or N,
L_{E}, T_{E}
are, independently of one another, CO, CO—NR_{E}¹², NR_{E}¹²—CO, sulfur, SO, SO₂, SO₂—NR_{E}¹², NR_{E}¹²—SO₂, CS, CS—NR_{E}¹², NR_{E}¹²—CS, CS—O, O—CS, CO—O, O—CO, oxygen, ethynylene, CR_{E}¹³—O—CR_{E}¹⁴, C(═CR_{E}¹³R_{E}¹⁴),
CR_{E}¹³═CR_{E}¹⁴, —CR_{E}¹³(OR_{E}¹⁵)—CHR_{E}¹⁴—, —CHR_{E}¹³—CR_{E}¹⁴(OR_{E}¹⁵)—,
R_{E}¹² is hydrogen, a branched or unbranched, optionally substituted C₁-C₆-alkyl, C₂-C₆-alkenyl, C₂-C₈-alkynyl, an optionally substituted C₃-C₇-cycloalkyl, hetaryl, arylalkyl or hetarylalkyl radical or a CO—R_{E}¹⁶, COOR_{E}¹⁶ or SO₂—R_{E}¹⁶ radical,
R_{E}¹³, R_{E}¹⁴
are, independently of one another, hydrogen, a hydroxyl group, a branched or unbranched, optionally substituted C₁-C₆-alkyl, C₁-C₄-alkoxy, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,
R_{E}¹⁵ is hydrogen, a branched or unbranched, optionally substituted C₁-C₆-alkyl, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,
R_{E}¹⁶ is hydrogen, a hydroxyl group, a branched or unbranched, optionally substituted C₁-C₆alkyl, C₂-C₆-alkenyl, C₂-C₆-alkynyl or C₁-C₅-alkylene-C₁-C₄-alkoxy radical, or an optionally substituted aryl, heterocycloalkyl, heterocycloalkenyl, hetaryl, C₃-C₇-cycloalkykl-, C₁-C₄-alkylene-C₃-C₇-cycloalkyl, arylalkyl, C₁-C₄-alkylene-C₃-C₇-heterocycloalkyl, C₁-C₄-alkylene-C₃-C₇-heterocycloalkenyl or hetarylalkyl radical and
Q_{E} is an optionally substituted 4- to 11-membered mono- or polycyclic aliphatic or aromatic hydrocarbon which may contain up to 6 double bonds and up to 6 identical or different heteroatoms selected from the group N, O or S, it being possible for the ring carbons or ring nitrogens optionally to be substituted.

5. The use of the structural element for the fomula I_{GL}
-G-L I_{GL}
for preparing compounds which bind to integrin receptors,
where G and L have the following meanings:
R_{G}⁴* is an R_{G}⁴ radical independent of R_{G}⁴.

6. A drug comprising the structural element of the formula I_{GL}
-G-L I_{GL}
where G and L have the following meanings:
L is a structural element of the formula I_{L} 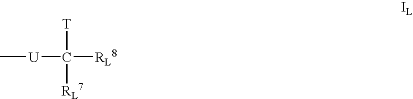
where
T is a COOH group or a radical which can be hydrolyzed to COOH and
—U—is —(CR_{L}¹R_{L}²)ₐ—(V_{L})_{b}—(CR_{L}³R_{L}⁴)_{c}—(W_{L})_{d}—(CR_{L}⁵R_{L}⁶)—(X_{L})_{f}
where
a, c, e
are, independently of one another, 0, 1, 2 or 3,
b, d, f
are, independently of one another, 0 or 1,
R_{L}¹, R_{L}², R_{L}³, R_{L}⁴, R_{L}⁵, R_{L}⁶
are, independently of one another, hydrogen, halogen, a hydroxyl group, a branched or unbranched, optionally substituted C₁-C₆-alkyl, C₂-C₆-alkenyl, C₂-C₆-alkynyl or C₁-C₆-alkylene-C₃-C₇-cycloalkyl radical, a radical —(CH₂)_{w}—(Y_{L})_{y}—R_{L}⁹, an optionally substituted C₃-C₆-cycloalkyl, aryl, arylalkyl, hetaryl or hetarylalkyl radical or, in each case independently of one another, two radicals R_{L}¹ and R_{L}² or R_{L}⁵ and R_{L}⁴ or R_{L}⁶ and R_{L}⁶ together are a 3- to 7-membered, optionally substituted, saturated or unsaturated carbocyclic or heterocyclic system which may contain up to three heteroatoms from the group of O, N or S,
w is 0, 1, 2, 3 or 4,
y is 0 or 1
Y_{L} is —CO—, —CO—N(R_{y}¹′, —N(R_{y}¹)—CO—N(R_{y}¹)—CO—N(R_{y}¹*)—, —N(R_{y}¹)—CO—O—, —O—, —S—, —SO₂, —SO₂—N(R_{y}¹)—, —SO₂, —CO—O—, —O—CO—, —O—CO—N(R_{y}¹)—, —N(R_{y}¹)— or —N(R_{y}¹)—SO₂—,
R_{y}^{1,} R_{y}^{1*}
are, independently of one another, hydrogen, a branched or unbranched, optionally substituted C₁-C₆-alkyl, C₁-C₆-alkoxyalkyl, C₂-C₆-alkenyl, C₂-C₁₂-alkynyl, CO-C₁-C₆-alkyl, CO—O—C₁-C₆-alkyl or SO₂-C₁-C₅-alkyl radical or an optionally substituted C₃-C₇-cycloalkyl, aryl, arylalkyl, CO—O-alkylene-aryl, CO-alkylene-aryl, CO-aryl, SO₂-aryl, hetaryl, CO-hetaryl or SO₂-alkylene-aryl radical,
R_{L}⁷, R_{L}⁸
are, independently of one another, hydrogen, halogen, a hydroxyl group, a branched or unbranched, optionally substituted C₁-C₆-alkyl, C₂-C₆-alkenyl, C₂-C₆-alkynyl or C₁-C₆-alkylene-C₃-C₇-cycloalkyl radical, an optionally substituted —(CH₂)_{w}—R_{L}^{9*} radical, an optionally substituted C₃-C₇-cycloalkyl, aryl, arylalkyl, hetaryl or hetarylalkyl radical or the radicals R_{L}⁷ and R_{L}⁸ together are a 3- to 7-membered, optionally substituted, saturated or unsaturated carbocyclic or heterocyclic system which may contain up to three heteroatoms from the group of O, N or S,
R_{L}⁹ is hydrogen, a hydroxyl group, CN, halogen, a branched or unbranched, optionally substituted C₁-C₆-alkyl radical, an optionally substituted C₃-C₇-cycloalkyl, aryl, heteroaryl or arylalkyl radical, an optionally C₁-C₄-alkyl- or aryl-substituted C₂-C₆-alkynyl or C₂-C₆alkenyl radical, an optionally substituted C₆-C₁₂-bicycloalkyl, C₁-C₆-alkylene-C₆-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three Identical or different radicals and which may contain up to three different or identical heteroatoms O, N, S, it being possible for two radicals together to be a fused, saturated, unsaturated or aromatic carbocyclic or heterocyclic system which may contain up to three, different or identical heteroatoms O, N, S, and it being possible for the cyclic system to be optionally substituted, or for another, optionally substituted, saturated, unsaturated or aromatic cyclic system to be fused to this cyclic system, or the radical R_{L}⁹ forms together with R_{y}¹ or R_{y}¹* a saturated or unsaturated C₃-C₇-heterocycle which may optionally contain up to two further heteroatoms selected from the group of O, S, or N,
R_{L}⁹* is hydrogen, a hydroxyl group, CN, halogen, a branched or unbranched, optionally substituted C₁-C₆-alkyl radical, an optionally substituted C₃-C₇-cycloalkyl, aryl, heteroaryl or arylalkyl radical, an optionally C₁-C₄-alkyl- or aryl-substituted C₂-C₆-alkynyl or C₂-C₆-alkenyl radical, an optionally substituted C₆-C₁₂-bicycloalkyl, C₁-C₆-alkylene-C₇-C₁₂-bicycloalkyl, C₇-C₂₀-tricycloalkyl or C₁-C₆-alkylene-C₇-C₂₀-tricycloalkyl radical, or a 3- to 8-membered, saturated or unsaturated heterocyclic system which is substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, N, and it being possible for the cyclic system to be optionally substituted, or for another, optionally substituted, saturated, unsaturated or aromatic cyclic system to be fused to this cyclic system,
W_{L} is an optionally substituted 4- to 11-membered mono- or polycyclic aliphatic or aromatic hydrocarbon which may contain up to 6 double bonds and up to 6 heteroatoms selected from the group of N, O, S,
V_{L,} X_{L}
are, independently of one another, —CO—, —CO—NR_{L}¹⁰—, —NR_{L}¹⁰—CO—, —S—, —SO—, —SO₂—, —SO₂—NR_{L}¹⁰—, —NR_{L}¹⁰—SO₂—, —CS—, —CS—NR_{L}¹⁰—, —NR_{L}¹⁰—CS—, —CS—O—, —O—CS—, —CO—O—, —O—CO—, —O—, ethynylene, —CHR_{L}¹¹—O—CHR_{L}¹²—, —C(═CR_{L}¹¹R_{L}¹²)—, —CR_{L}¹¹═CR_{L}¹²—, —CR_{L}¹¹(OR_{L}¹³)—CHR_{L}¹²—, CHR_{L}¹¹—CR_{L}¹²(OR_{L}¹³)—, —CH(NR_{L}¹⁴—SO₂—R_{L}¹⁵)—, —CH(NR_{L}¹⁴—CO—R_{L}¹⁵), —CH(NR_{L}¹⁴—CO—OR_{L}¹⁶)—, CH(NR_{L}¹⁴—CO—NR_{14′}R_{L}¹⁵)—, —CH(CO—R_{L}¹⁵)—, —CH(CO—OR_{L}¹⁶)— or CH(CO—NR_{L}¹⁴R_{L}¹⁵)—,
R_{L}¹⁰ is hydrogen, a branched or unbranched, optionally substituted C₁C₆-alkyl, C₁-C₆-alkoxyalkyl, C₂-C₆-alkenyl, C₃-C₁₂-alkynyl, CO-C₁-C₆-alkyl, CO—O-C₁-C₆-alkyl or SO₂-C₁-C₆-alkyl radical or an optionally substituted C₃-C₇-cycloalkyl, CO—O-alkylene-aryl, CO-alkylene-aryl, arylalkyl-, CO-aryl, SO₂-aryl, hetaryl, CO-hetaryl, hetarylalkyl or SO₂-alkylene-aryl radical, or R_{L}¹⁰ and a radical selected from the group of R_{L}¹, R_{L}², R_{L}³, R_{L}⁴, R_{L}⁵, R_{L}⁶ or R_{L}⁶ together are an optionally substituted 4- to 8-membered heterocycle which may contain up to five identical or different heteroatoms O, N or S,
R_{L}^{11,} R_{L}¹²
are, independently of one another, hydrogen, a hydroxyl group, a branched or unbranched, optionally substituted C₁-C₆-alkyl, C₁-C₄-alkoxy, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,
R_{L}¹³ is hydrogen, a branched or unbranched, optionally substituted C₁-C₆-alkyl, C₂-C₆-alkenyl, C₂-C₆-alkynyl or alkylene-cycloalkyl radical or an optionally substituted C₃-C₇-cycloalkyl, aryl, arylalkyl, hetaryl or hetarylalkyl radical,
R_{L}¹⁴, R_{L}^{14,}
are, independently of one another, hydrogen, a branched or unbranched, optionally substituted C₁-C₆-alkyl, C₁-C₆-alkoxyalkyl, C₁-C₆-alkenyl, C₃-C₁₂-alkynyl, CO-C₁-C₆-alkyl, CO—O—C₁-C₆-alkyl, C₁-C₆-alkylene-C₃-C₇-cycloalkyl, or SO₂-C₁-C₆-alkyl radical or an optionally substituted C₃-C₇-cycloalkyl, CO—O-alkylene-aryl, CO-alkylene-aryl, aryl, CO-aryl, SO₂-aryl, hetaryl, CO-hetaryl, hetarylalkyl, arylalkyl or SO₂-alkylene-aryl radical,
R_{L}¹⁵ is a branched or unbranched, optionally substituted C₁-C₈-alkyl, alkoxyalkyl, C₁-C₆-alkylene-C₃-C₇-cycloalkyl radical, C₆-C₁₂-bicycloalkyl, C₁-C₆-akylene-C₆-C₁₂-bicycloalkyl, C₇-C₂₀-tricycloalkyl or C₁-C₆-alkylene-C₇-C₂₀-tricycloalkyl radical, a C₃-C₇-cycloalkyl, aryl, arylalkyl, hetarylalkyl or 3- to 8-membered, saturated, unsaturated or aromatic heterocyclic radical which may be substituted by up to three identical or different radicals and which may contain up to three different or identical heteroatoms O, N, S, it being possible for two radicals together to be a fused, saturated, unsaturated or aromatic carbocyclic or heterocyclic system which may contain up to three different or identical heteroatoms O, N, S, and it being possible for this cyclic system to be optionally substituted, or for another, optionally substituted, saturated, unsaturated or aromatic cyclic system to be fused to this cyclic system, or the radical R_{L}¹⁵ forms together with R_{L}¹⁴ or R_{L}¹⁴* a saturated or unsaturated C₃-C₇ heterocycle which may optionally contain up to two further heteroatoms selected from the group of O, S or N, and
R_{L}¹⁶ is a branched or unbranched, optionally substituted C₁-C₆-alkyl, alkoxyalkyl or C₁-C₆-alkylene-C₃-C₇-cycloalkyl radical or an optionally substituted C₃-C₇-cycloalkyl, aryl, arylalkyl, hetaryl or hetarylalkyl radical,
G is a structural element of the formula IG 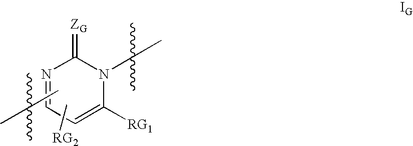
where
the structural element G can be incorporated in both orientations, and
Z_{G} is oxygen, sulfur or NR_{G}³,
R_{G}^{1,} R_{G}²
are, independently of one another, hydrogen, CN, NO₂, halogen, a branched or unbranched, optionally substituted C₁-C₆-alkyl, C₂-C₆-alkenyl or C₂-C₆-alkynyl radical, a branched or unbranched, optionally substituted C₁-C₄-alkylene-OR_{G}⁴, C₁-C₄-alkylene-CO—OR_{G}⁴, C₁-C₄-alkylene-CO—R_{G}⁴, C₁-C₄-alkylene-SO₂—NR_{G}⁵R_{G}⁶, C₁-C₄-alkylene-CO—NR_{G}⁵R_{G}⁶, C₁-C₄-alkylene-NR_{G}⁶R_{G}⁶ or C₁-C₄-alkylene-SR_{G}⁴ radical, an optionally substituted C₃-C₇-cycloalkyl, C₁-C₄-alkylene-C₃-C₇-cycloalkyl, C₁-C₄-alkylene-C₃-C₇-heterocycloalkyl or C₁-C₄-alkylene-C₃-C₇-heterocyclo-alkenyl radical, an optionally substituted aryl, arylalkyl, hetaryl or hetarylalkyl radical, an —S—R_{G}⁴, —O—R_{G}⁴, —SO—R_{G}⁴, —SO₂—R_{G}⁴, —CO—OR_{G}⁴, —O—CO—R_{G}⁴, —O—CO—NR_{G}⁵R_{G}⁶, —SO₂—NR_{G}⁶R_{G}⁶, —CO—NR_{G}⁵R_{G}⁶, —NR_{G}⁵R_{G}⁶; CO—R_{G}⁴ radical, or R_{G}¹ and R_{G}² together are an optionally substituted, saturated, unsaturated or aromatic 3- to 9-membered carbocyclic, polycarbocyclic, heterocyclic or polyheterocyclic system which may contain up to 4 heteroatoms selected from the group of O, N, S,
R_{G}³ is hydrogen, a hydroxyl group, CN, a branched or unbranched, optionally substituted C₁-C₆-alkyl or C₁-C₄-alkoxy radical or an optionally substituted C₃-C₇-cycloalkyl, —O—C₃-C₇-cycloalkyl radical, aryl-, —O-aryl, arylalkyl or —O-alkylene aryl radical,
R_{G}⁴ is hydrogen, a branched or unbranched, optionally substituted C₁-C₈-alkyl, C₂-C₆-alkenyl, C₂-C₆alkynyl, C₁-C₅-alkylene-C₁-C₄-alkoxy-, mono- and bisalkylaminoalkylene or acylaminoalkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-heterocycloalkyl, C₁-C₄-alkylene-heterocycloalkenyl or hetarylalkyl radical,
R_{G}⁵, R_{G}⁸ are, independently of one another, hydrogen, a branched or unbranched, optionally substituted C₁-C₈-alkyl, C₂-C₆alkenyl, C₂-C₆-alkynyl, C₁-C₅-alkylene-C₁-C₄-alkoxy, mono and bisalkylaminoalkylene or acylamino-alkylene radical or an optionally substituted aryl, heterocycloalkyl, heterocycloalkenyl, hetaryl, C₃-C₇-cycloalkyl, C₁-C₄-alkylene-C₃-C₇-cycloalkyl, arylalkyl, C₁-C₄-alkylene-heterocycloalkyl, C₁-C₄-alkylene-heterocycloalkenyl or hetarylalkyl radical, or an —SO₂—R_{G}⁴, —CO—OR_{G}⁴, —CO—NR_{G}⁴R_{G}⁴* or —CO—R_{G}⁴ radical, and
R_{G}⁴* is an R_{G}⁴ radical independent of R_{G}⁴.

7. A pharmaceutical preparation comprising at least one compound as claimed in claim 1 in addition to conventional pharmaceutical excipients.

8. A compound as claimed in claim 1 for use as a drug.

9. The use of the compounds as claimed in claim 1 for producing drugs for the treatment of diseases.

10. The use of the compounds as claimed in claim 1 as integrin receptor ligands.

11. The use as claimed in claim 10 as ligands of the αvβ2 integrin receptor.

12. The use as claimed in claim 9 for producing drugs for the treatment of diseases in which the interaction between integrins and their natural ligands is increased or decreased.

13. The use as claimed in claim 12 for the treatment of diseases in which the interaction between αvβ₃ integrin and its natural ligand is increased or decreased.

14. The use as claimed in claim 13 for the treatment of atherosclerosis, rheumatoid arthritis, restenosis after vessel injury or stent implantation, angioplasty, acute kidney failure, angioqenesis associated microangiopathies, diabetic angiopathies, vascular occlusion mediated by blood platelets, arterial thrombosis, congestive heart failure, myocardial infarction, stroke, cancer, osteoporosis, high blood pressure, psoriasis or viral, fungal, parasitic or bacterial diseases, inflammations, wound healing, hyperparathyroidism, Paget's disease, malignant hypercalcemia or metastatic osteolytic lesions or for antiviral, antimycotic, antiparasitic or antibacterial therapy and prophylaxis.

15. A pharmaceutical preparation comprising at least one compound as claimed in claim 1, where appropriate pharmaceutical excipients and at least one other compound selected from the group of inhibitors of blood platelet adhesion, activation or aggregation, anticoagulants which impede thrombin activity or formation, antagonists of blood platelet-activating compounds, or selection antagonists.

16. The use of the pharmaceutical preparation as claimed in claim 15 for producing a drug for the treatment of vascular occlusion mediated by blood platelets or thrombosis.

17. A pharmaceutical preparation comprising at least one compound as claimed in claim 1, where appropriate pharmaceutical excipients and at least one other compound selected from the group of
inhibitors of blood platelet activation or aggregation,
serine protease inhibitors,
fibrinogen-reducing compounds,
selectin antagonists,
antagonists of ICAM-1 or VCAM-1
inhibitors of leukocyte adhesion
inhibitors of vessel wall transmigration,
fibrinolysis-modulating compounds,
inhibitors of complement factors,
endothelin receptor antagonists,
tyrosine kinase inhibitors,
antioxidants or
interleukin β antagonists.

18. The use of pharmaceutical preparation as claimed in claim 17 for producing a drug for the treatment of myocardial infarct or stroke.

19. A pharmaceutical preparation comprising at least one compound as claimed in claim 1, where appropriate pharmaceutical excipients and at least one other compound selected from the group of
endothelin antagonists,
ACE inhibitors,
angiotensin receptor antagonists,
endopeptidase inhibitors,
beta blockers,
calcium channel blockers,
phosphodiesterase inhibitors or
caspase inhibitors.

20. The use of the pharmaceutical preparation as claimed in claim 19 for producing a drug for the treatment of congestive heart failure.

21. A pharmaceutical preparation comprising at least one compound as claimed in claim 1, where appropriate pharmaceutical excipients and at least one other compound selected from the group of
thrombin inhibitors,
inhibitors of factor Xa,
inhibitors of the coagulation pathway leading to thrombin formation,
inhibitors of blood platelet adhesion, activation or aggregation,
endothelin receptor antagonists,
nitric oxide synthase inhibitors,
CD44 antagonists,
selectin antagonists,
MCP-1 antagonists,
inhibitors of signal transduction in proliferating cells,
antagonists of the cellular response mediated by EGF, PDGF, VEGF or bFGF or antioxidants.

22. The use of the pharmaceutical preparation as claimed in claim 21 for producing a drug for the treatment of restenosis after vessel injury or stent implantation.

23. A pharmaceutical preparation comprising at least one compound as claimed in claim 1, where appropriate pharmaceutical excipients and at least one other compound selected from the group of
inhibitors of MMPs,
selectin antagonists,
endothelin antagonists,
ACE inhibitors,
angiotensin receptor antagonists,
glycosylation inhibitors or
AGE formation inhibitors or AGE breakers and antagonists of their receptors.

24. The use of the pharmaceutical preparation as claimed in claim 23 for producing a drug for the treatment of diabetic angiopathies.

25. A pharmaceutical preparation comprising at least one compound as claimed in claim 1, where appropriate pharmaceutical excipients and at least one other compound selected from the group of
lipid-lowering compounds,
selectin antagonists,
antagonists of ICAM-1 or VCAM-1
heparin or low molecular weight heparins or other GAGs,
inhibitors of MMPs,
endothelln antagonists,
apolipoprotein A1 antagonists,
cholesterol antagonists,
HMG-CoA reductase inhibtors,
ACAT inhibitors,
ACE inhibitors,
angiotensin receptor antagonists,
tyrosine kinase inhibitors,
protein kinase C inhibitors,
calcium channel blockers,
LDL receptor function stimulants,
antioxidants
LCAT mimetics or
free radical scavengers.

26. The use of the pharmaceutical preparation as claimed in claim 25 for producing a drug for the treatment of atherosclerosis.

27. A pharmaceutical preparation comprisng at least one compound as claimed in claim 1, where appropriate pharmaceutical excipients and at least one other compound selected from the group of
cytostatic or antineoplastic compounds,
compounds which inhibit proliferation or
heparin or low molecular weight heparins or other GAGs.

28. The use of the pharmaceutical preparation as claimed in claim 27 for producing a drug for the treatment of cancer.

29. A pharmaceutical preparation comprising at least one compound as claimed in claim 1, where appropriate pharmaceutical excipients and at least one other compound selected from the group of
compounds for antiresorptive therapy,
compounds for hormone replacement therapy,
recombinant human growth hormone,
bisphosphonates,
compounds for calcitonin therapy,
calcitonin stimulants,
calcium channel blockers,
bone formation stimulants,
interleukin-6 antagonists or
Src tyrosine kinase inhibitors.

30. The use of the pharmaceutical preparation as claimed in claim 29 for producing a drug for the treatment of osteoporosis.

31. A pharmaceutical preparation comprising at least one compound as claimed in claim 1, where appropriate pharmaceutical excipients and at least one other compound selected from the group of
TNF antagonists,
antagonists of VLA-4 or VCAM-1,
antagonists of LFA-1, Mac-1 or ICAMs,
complement inhibitors,
immunosuppressants,
interleukin-1, -5 or -8 antagonists or
dihydrofolate reductase-inhibitors.

32. The use of the pharmaceutical preparation as claimed in claim 31 for producing a drug for the treatment of rheumatoid arthritis.

33. A pharmacetucal preparation camprisinq at least one compound as claimed in claim 1, where appropriate pharmaceutical excipients and at least one other compound selected from the group of
coliagenase,
PDGF antagonists or
MMPs.

34. The use of the pharmaceutical preparation as claimed in claim 33 for producing a drug for improving wound healing.
